SEQUENCE LISTING (89 sequences)

SEQ ID NO: 1 - DNA, 34 bp, Artificial Sequence
  L3
ataacttcgtataaagtctcctatacgaagttat

SEQ ID NO: 2 - DNA, 34 bp, Artificial Sequence
  2L
ataacttcgtatagcatacattatacgaagttat

SEQ ID NO: 3 - DNA, 34 bp, Artificial Sequence
  loxFas
ataacttcgtatatacctttctatacgaagttat

SEQ ID NO: 4 - DNA, 608 bp, Human cytomegalovirus
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattagcatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctccgtttagtgaacgtcagatc

SEQ ID NO: 5 - DNA, 696 bp, Human cytomegalovirus
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattagcatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctccgtttagtgaacgtcagatctagctctgggagaggagcccagcactagaagtcggcggtgtttccattcggtgatcagcactgaacacagaggaagcttgccgccacc

SEQ ID NO: 6 - DNA, 2125 bp, Human cytomegalovirus
ctgcagtgaataataaaatgtgtgtttgtccgaaatacgcgttttgagatttctgtcgccgactaaattcatgtcgcgcgatagtggtgtttatcgccgatagagatggcgatattggaaaaatcgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgccacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccacgcgaatctcgggtacgtgttccggacatgggctcttctccggtagcggcggagcttctacatccgagccctgctcccatgcctccagcgactcatggtcgctcggcagctccttgctcctaacagtggaggccagacttaggcacagcacgatgcccaccaccaccagtgtgccgcacaaggccgtggcggtagggtatgtgtctgaaaatgagctcggggagcgggcttgcaccgctgacgcatttggaagacttaaggcagcggcagaagaagatgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttgacacggtttaaacgccgccacc

SEQ ID NO: 7 - DNA, 129 bp, Simian virus 40
aacttgtttattgcagcttataatggttacaaataaagcaatagcatcacaaatttcacaaataaagcatttttttcaccattctagttgtggtttgtccaaactcatcaatgtatcttatcatgtctg

SEQ ID NO: 8 - DNA, 225 bp, Bos taurus
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 9 - DNA, 73 bp, Homo sapiens
caggataatatatggtagggttcatagccagagtaacctttttttttaatttttattttattttatttttgag

SEQ ID NO: 10 - DNA, 288 bp, Simian virus 40
agtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcg

SEQ ID NO: 11 - DNA, 798 bp, Artificial Sequence
  green fluorescent protein encoding nucleic acid
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgagctcaagcttcgaattctgcagtcgacggtaccgcgggcccgggatccaccggatctagatga

SEQ ID NO: 12 - DNA, 20 bp, Artificial Sequence
  SIRT-1 guide RNA
tatcatccaactcaggtgga

SEQ ID NO: 13 - DNA, 20 bp, Artificial Sequence
  SIRT-1 guide RNA
gcagcatctcatgattggca

SEQ ID NO: 14 - DNA, 20 bp, Artificial Sequence
  SIRT-1 guide RNA
gcattcttgaagtaacttca

SEQ ID NO: 15 - DNA, 20 bp, Artificial Sequence
  MYC guide RNA
ctatgacctcgactacgact

SEQ ID NO: 16 - DNA, 20 bp, Artificial Sequence
  MYC guide RNA
ggacgcagcgaccgtcacat

SEQ ID NO: 17 - DNA, 20 bp, Artificial Sequence
  MYC guide RNA
caccatctccagctgatccg

SEQ ID NO: 18 - DNA, 20 bp, Artificial Sequence
  STK11 guide RNA
cagccacccgagatcgccaa

SEQ ID NO: 19 - DNA, 20 bp, Artificial Sequence
  STK11 guide RNA
gacacctgccggacgagcca

SEQ ID NO: 20 - DNA, 20 bp, Artificial Sequence
  STK11 guide RNA
ccaggccgtcaatcagctgg

SEQ ID NO: 21 - DNA, 20 bp, Artificial Sequence
  SMAD4 guide RNA
ctgcctgccagaatactggc

SEQ ID NO: 22 - DNA, 20 bp, Artificial Sequence
  SMAD4 guide RNA
tctgcaacagtccttcacta

SEQ ID NO: 23 - DNA, 20 bp, Artificial Sequence
  SMAD4 guide RNA
gtaacaatagggcagcttga

SEQ ID NO: 24 - DNA, 20 bp, Artificial Sequence
  PPP2CB guide RNA
gagcgtattacaatattgag

SEQ ID NO: 25 - DNA, 20 bp, Artificial Sequence
  PPP2CB guide RNA
tgtaaagtatttccatacgt

SEQ ID NO: 26 - DNA, 20 bp, Artificial Sequence
  PPP2CB guide RNA
ccatctactaaagctgtaag

SEQ ID NO: 27 - DNA, 20 bp, Artificial Sequence
  RBM38 guide RNA
aggtgcctggtactgcacga

SEQ ID NO: 28 - DNA, 20 bp, Artificial Sequence
  RBM38 guide RNA
atatgggtactggtcgtagg

SEQ ID NO: 29 - DNA, 20 bp, Artificial Sequence
  RBM38 guide RNA
cgtatattcaaggtagggcg

SEQ ID NO: 30 - DNA, 20 bp, Artificial Sequence
  NF1 guide RNA
aataattcaggatatatcca

SEQ ID NO: 31 - DNA, 20 bp, Artificial Sequence
  NF1 guide RNA
aatttgcagtggccaaactg

SEQ ID NO: 32 - DNA, 20 bp, Artificial Sequence
  NF1 guide RNA
ccaaactgcggctttacgtt

SEQ ID NO: 33 - DNA, 20 bp, Artificial Sequence
  CDK12 guide RNA
actatgaccttagccccccg

SEQ ID NO: 34 - DNA, 20 bp, Artificial Sequence
  CDK12 guide RNA
ttagcaagtctcgggaccgc

SEQ ID NO: 35 - DNA, 20 bp, Artificial Sequence
  CDK12 guide RNA
gcttgtgcttcgacaccaag

SEQ ID NO: 36 - DNA, 20 bp, Artificial Sequence
  SIN3A guide RNA
attctgtgagaaatgaccat

SEQ ID NO: 37 - DNA, 20 bp, Artificial Sequence
  SIN3A guide RNA
acgtctcttcaaaaaccagg

SEQ ID NO: 38 - DNA, 20 bp, Artificial Sequence
  SIN3A guide RNA
ttttgaagagacgtgccacc

SEQ ID NO: 39 - DNA, 20 bp, Artificial Sequence
  PARP-1 guide RNA
ttgctttgtcaagaaccggg

SEQ ID NO: 40 - DNA, 20 bp, Artificial Sequence
  PARP-1 guide RNA
tatagtgccagccagctcaa

SEQ ID NO: 41 - DNA, 20 bp, Artificial Sequence
  PARP-1 guide RNA
cggttcttgacaaagcaagt

SEQ ID NO: 42 - DNA, 20 bp, Artificial Sequence
  ATM guide RNA
cttctacctcaacaacgtcg

SEQ ID NO: 43 - DNA, 20 bp, Artificial Sequence
  ATM guide RNA
tcacagttaggtaaactgga

SEQ ID NO: 44 - DNA, 20 bp, Artificial Sequence
  ATM guide RNA
atatgtgttacgatgcctta

SEQ ID NO: 45 - DNA, 20 bp, Artificial Sequence
  Hipk2 guide RNA
tggtagagaaggcggaccga

SEQ ID NO: 46 - DNA, 20 bp, Artificial Sequence
  Hipk2 guide RNA
ataggtcaatgaattcccgt

SEQ ID NO: 47 - DNA, 20 bp, Artificial Sequence
  Hipk2 guide RNA
gtgtcattgtgacaaagggg

SEQ ID NO: 48 - DNA, 20 bp, Artificial Sequence
  BARD1 guide RNA
gcttgcagaaaatatactgt

SEQ ID NO: 49 - DNA, 20 bp, Artificial Sequence
  BARD1 guide RNA
tagctgagatcaacaagaag

SEQ ID NO: 50 - DNA, 20 bp, Artificial Sequence
  BARD1 guide RNA
catctaaccttcttacttcg

SEQ ID NO: 51 - DNA, 20 bp, Artificial Sequence
  HIF1AN guide RNA
tgtgtaccctgctctgaagt

SEQ ID NO: 52 - DNA, 20 bp, Artificial Sequence
  HIF1AN guide RNA
cttcaaaccaaggtccagca

SEQ ID NO: 53 - DNA, 20 bp, Artificial Sequence
  HIF1AN guide RNA
acaggatatacagcatcgag

SEQ ID NO: 54 - DNA, 20 bp, Artificial Sequence
  SMAD3 guide RNA
ggtcaggccatcgccacagg

SEQ ID NO: 55 - DNA, 20 bp, Artificial Sequence
  SMAD3 guide RNA
ggcaaactcacatagctcca

SEQ ID NO: 56 - DNA, 20 bp, Artificial Sequence
  SMAD3 guide RNA
gccgggatctcggtgtggcg

SEQ ID NO: 57 - DNA, 20 bp, Artificial Sequence
  CDKN1A guide RNA
gagaggttccgggtccaccg

SEQ ID NO: 58 - DNA, 20 bp, Artificial Sequence
  CDKN1A guide RNA
accgttctcgggcctcctgg

SEQ ID NO: 59 - DNA, 20 bp, Artificial Sequence
  CDKN1A guide RNA
ccacgggaccgaagagacgg

SEQ ID NO: 60 - DNA, 18 bp, Artificial Sequence
  MYC verfication primer forward
cacacacacacttggaag

SEQ ID NO: 61 - DNA, 18 bp, Artificial Sequence
  MYC verfication primer reverse
cttgatgaaggtctcgtc

SEQ ID NO: 62 - DNA, 20 bp, Artificial Sequence
  STK11 verfication primer  forward
ctagagaaaacccacagttc

SEQ ID NO: 63 - DNA, 18 bp, Artificial Sequence
  STK11 verfication primer reverse
tctggccttctaattgtc

SEQ ID NO: 64 - DNA, 18 bp, Artificial Sequence
  SMAD4 verfication primer forward
taggtgtgtatggtgcag

SEQ ID NO: 65 - DNA, 19 bp, Artificial Sequence
  SMAD4 verfication primer reverse
aggtcttctcctagtgctc

SEQ ID NO: 66 - DNA, 21 bp, Artificial Sequence
  PPP2CB verfication primer forward
cttgtaaatacagatcctgag

SEQ ID NO: 67 - DNA, 19 bp, Artificial Sequence
  PPP2CB verfication primer reverse
cccacaagattactctagc

SEQ ID NO: 68 - DNA, 18 bp, Artificial Sequence
  RBM38 verfication primer forward
tctcatgtccttcctcag

SEQ ID NO: 69 - DNA, 18 bp, Artificial Sequence
  RBM38 verfication primer reverse
gttttgtagatggggttg

SEQ ID NO: 70 - DNA, 18 bp, Artificial Sequence
  NF1 verfication primer forward
acagagctaagagccttc

SEQ ID NO: 71 - DNA, 23 bp, Artificial Sequence
  NF1 verfication primer reverse
ctgtaagaccctaatagtatgac

SEQ ID NO: 72 - DNA, 20 bp, Artificial Sequence
  CDK12 verfication primer forward
caggactcttcttgtaggag

SEQ ID NO: 73 - DNA, 18 bp, Artificial Sequence
  CDK12 verfication primer reverse
gattcagacaccttctcc

SEQ ID NO: 74 - DNA, 18 bp, Artificial Sequence
  SIN3A verfication primer forward
gtggcctatactaacgtg

SEQ ID NO: 75 - DNA, 18 bp, Artificial Sequence
  SIN3A verfication primer reverse
ctcccttagtgtgtatcg

SEQ ID NO: 76 - DNA, 18 bp, Artificial Sequence
  PARP-1 verfication primer forward
ctctctgcagttccctac

SEQ ID NO: 77 - DNA, 18 bp, Artificial Sequence
  PARP-1 verfication primer reverse
atgtaagtgcaaggtgtc

SEQ ID NO: 78 - DNA, 19 bp, Artificial Sequence
  ATM verfication primer forward
gtaaagagctagccagaag

SEQ ID NO: 79 - DNA, 18 bp, Artificial Sequence
  ATM verfication primer reverse
gaaggtttacaggctgag

SEQ ID NO: 80 - DNA, 18 bp, Artificial Sequence
  Hipk2 verfication primer forward
acgtacgtatgtgaatcc

SEQ ID NO: 81 - DNA, 20 bp, Artificial Sequence
  Hipk2 verfication primer reverse
ggtaaactacagtcttaggc

SEQ ID NO: 82 - DNA, 18 bp, Artificial Sequence
  BARD1 verfication primer forward
ggctaagggagttatctg

SEQ ID NO: 83 - DNA, 18 bp, Artificial Sequence
  BARD1 verfication primer reverse
caacacatctaggacagg

SEQ ID NO: 84 - DNA, 18 bp, Artificial Sequence
  HIF1AN verfication primer forward
gttcagtaatggaaccag

SEQ ID NO: 85 - DNA, 18 bp, Artificial Sequence
  HIF1AN verfication primer reverse
ctcatctctatggtgtgc

SEQ ID NO: 86 - DNA, 19 bp, Artificial Sequence
  SMAD3 verfication primer forward
acttcactgacaccttctg

SEQ ID NO: 87 - DNA, 18 bp, Artificial Sequence
  SMAD3 verfication primer reverse
gaacaacgacatggagag

SEQ ID NO: 88 - DNA, 18 bp, Artificial Sequence
  CDKN1A verfication primer forward
tacctgtccctacctgtc

SEQ ID NO: 89 - DNA, 19 bp, Artificial Sequence
  CDKN1A verfication primer reverse
gggaagattgtgacttatg

(19) United States
(12) Patent Application Publication
(10) Pub. No.: US 2022/0154207 A1
(43) Pub. Date: May 19, 2022

(54) MAMMALIAN CELL LINES WITH GENE KNOCKOUT

(71) Applicant: Hoffmann-La Roche Inc., Little Falls, NJ (US)

(72) Inventors: Simon Auslaender, Wolfratshausen (DE); Benedikt Oswald, Munich (DE); Niels Bauer, Penzberg (DE)

(73) Assignee: Hoffmann-La Roche Inc., Little Falls, NJ (US)

(21) Appl. No.: 17/483,587

(22) Filed: Sep. 23, 2021

(30) Foreign Application Priority Data
Sep. 24, 2020 (EP) 20197946.5

(51) Int. Cl. C12N 15/85; C12N 15/10

(52) U.S. Cl. CPC C12N 15/85; C12N 15/102

(57) ABSTRACT

Herein is reported a method for generating a recombinant mammalian cell expressing a heterologous polypeptide and a method for producing a heterologous polypeptide using said recombinant mammalian cell, wherein in the recombinant cell the expression of at least the endogenous gene MYC has been reduced. It has been found that the knockout of at least the endogenous gene MYC in mammalian cells, e.g. such as CHO cells, improves recombinant productivity by the cells.

### CROSS-REFERENCE TO RELATED APPLICATIONS

This application claims priority to European Patent Application No. 20197946.5 filed Sep. 24, 2020, all of which are incorporated by reference in its entirety.

### SEQUENCE LISTING

This application contains a Sequence Listing which has been submitted electronically in ASCII format and is hereby incorporated by reference in its entirety. Said ASCII copy, created on Sep. 20, 2021, is named P36383-US_SeqList.txt and is 23,419 bytes in size.

### FIELD OF INVENTION

The current invention is in the field of cell line development for the recombinant production of therapeutic polypeptides, such as therapeutic antibodies. In more detail, herein is reported a mammalian cell with a functional knockout of at least one endogenous gene, which results in improved expression characteristics.

### BACKGROUND

Mammalian host cell lines, especially CHO and HEK cell lines, are used for the recombinant production of secreted proteins, such as supply proteins (e.g. antigens, receptors and others) and therapeutic molecules (e.g. antibodies, cytokines and others). These host cell lines are transfected with vectors comprising the expression cassettes encoding the corresponding therapeutic molecule. Subsequently stable transfectants are selected by applying selective pressure. This results in a cell pool consisting of individual clones. In a single cell-cloning step, these clones are isolated and subsequently screened with different assays to identify top producer cells.

Genetic engineering approaches have been applied to host cell lines in order to improve their characteristics, such as (i) overexpression of endogenous proteins involved in the unfolded protein response pathway to improve protein folding and secretion (Gulis, G., et al., BMC biotechnology, 14 (2014) 26), (ii) overexpression of anti-apoptotic proteins to improve cell viability and prolong the fermentation process (Lee, J. S., et al., Biotechnol. Bioeng. 110 (2013) 2195-2207), (iii) overexpression of miRNA and/or shRNA molecules to improve cell growth and productivity (Fischer, S., et al., J. Biotechnol. 212 (2015) 32-43), (iv) overexpression of glycoenzymes to modulate glycosylation pattern of therapeutic molecules (Ferrara, C., et al., Biotechnol. Bioeng. 93 (2006) 851-861) and many others (Fischer, S., et al., Biotechnol. Adv. 33 (2015) 1878-1896).

In addition, knockout of endogenous proteins has been shown to improve cell characteristics. Examples are (i) knockout of BAX/BAK proteins leading to increased apoptosis resistance (Cost, G. J., et al., Biotechnol. Bioeng. 105 (2010) 330-340), (ii) knockout of PUTS to produce non-fucosylated proteins (Yamane-Ohnuki, N., et al., Biotechnol. Bioeng. 87 (2004) 614-622), (iii) knockout of GS to increase selection efficiency using GS selection system (Fan, L., et al., Biotechnol. Bioeng. 109 (2012) 1007-1015) and many others (Fischer, S., et al., Biotechnol. Adv. 33 (2015) 1878-1896). While zinc finger or TALEN proteins are mainly used in the past, CRISPR/Cas9 recently has been established for versatile and simple targeting of genomic sequences for knockout purposes. For example, miRNA-744 was targeted in CHO cells using CRISPR/Cas9 by using multiple gRNA enabling sequence excision (Raab, N., et al., Biotechnol. J. (2019) 1800477).

US 2007/160586 discloses methods for extending the replicative lifespan of cells.

EP 3 308 778 discloses arginine and its use as a T-cell modulator.

Fischer, S., et al. disclose enhanced protein production by microRNA-30 family in CHO cells is mediated by the modulation of the ubiquitin pathway (J. Biotechnol. 212 (2015) 32-43).

Knockouts of single endogenous genes that increase productivity are highly desired because of its simplicity to be introduced in host cell lines.

### SUMMARY OF THE INVENTION

One independent aspect according to the invention is a method for generating a recombinant mammalian cell expressing a heterologous polypeptide and a method for producing a heterologous polypeptide using said recombinant mammalian cell, wherein in the recombinant mammalian cell the activity or function or expression of one or more, i.e. at least one, endogenous gene(s) selected from the group consisting of MYC, STK11, SMAD4, PPP2CB, RBM38, NF1, CDK12, SIN3A, PARP-1, ATM, Hipk2, BARD1, HIF1AN, SMAD3, PALB2, FUBP1, RBL2, RPS6KA3, GPS2, ETS1, E2F5, CDKN1A, RNF43, EEF2K, AKT1, BRCA1, ATR, SMAD2, BAD, FOXO1, PBRM1, NRAS, BRCA2, NOTCH1, CREBBP, and RBX1 has been reduced or eliminated or diminished or (completely) knocked-out.

The invention is based, at least in part, on the finding that the functional knockout of at least one of the genes from the group of genes consisting of MYC, STK11, SMAD4, PPP2CB, RBM38, NF1, CDK12, SIN3A, PARP-1, ATM, Hipk2, BARD1, HIF1AN, SMAD3, PALB2, FUBP1, RBL2, RPS6KA3, GPS2, ETS1, E2F5, CDKN1A, RNF43, EEF2K, AKT1, BRCA1, ATR, SMAD2, BAD, FOXO1, PBRM1, NRAS, BRCA2, NOTCH1, CREBBP, and RBX1 in a mammalian cell, e.g. such as a CHO cell, improves recombinant productivity, especially of complex antibody formats.

For the current invention the sequence of steps to generate the recombinant mammalian cell is not decisive, i.e. if the transgene is introduced prior to the functional knockout of at least one of the genes from the group consisting of MYC, STK11, SMAD4, PPP2CB, RBM38, NF1, CDK12, SIN3A, PARP-1, ATM, Hipk2, BARD1, HIF1AN, SMAD3, PALB2, FUBP1, RBL2, RPS6KA3, GPS2, ETS1, E2F5, CDKN1A, RNF43, EEF2K, AKT1, BRCA1, ATR, SMAD2, BAD, FOXO1, PBRM1, NRAS, BRCA2, NOTCH1, CREBBP, and RBX1, of if first the functional knockout is effected and thereafter the cell is transfected with the transgene. In one preferred embodiment of all aspects and embodiments, the transgene, i.e. the nucleic acid encoding the heterologous polypeptide, is introduced prior to the functional knock out of the endogenous gene. In certain preferred embodiments, the endogenous gene is the MYC gene.

One independent aspect of the current invention is a mammalian cell wherein the activity or/and function or/and expression of at least one endogenous gene selected from the group of genes consisting of MYC, STK11, SMAD4, PPP2CB, RBM38, NF1, CDK12, SIN3A, PARP-1, ATM, Hipk2, BARD1, HIF1AN, SMAD3, PALB2, FUBP1, RBL2, RPS6KA3, GPS2, ETS1, E2F5, CDKN1A, RNF43, EEF2K, AKT1, BRCA1, ATR, SMAD2, BAD, FOXO1, PBRM1, NRAS, BRCA2, NOTCH1, CREBBP, and RBX1 has been reduced or eliminated or diminished or (completely) knocked-out. In certain preferred embodiments, the endogenous gene is the MYC gene.

One independent aspect of the current invention is a mammalian cell, wherein the expression of at least one endogenous gene selected from the group of genes consisting of MYC, STK11, SMAD4, PPP2CB, RBM38, NF1, CDK12, SIN3A, PARP-1, ATM, Hipk2, BARD1, HIF1AN, SMAD3, PALB2, FUBP1, RBL2, RPS6KA3, GPS2, ETS1, E2F5, CDKN1A, RNF43, EEF2K, AKT1, BRCA1, ATR, SMAD2, BAD, FOXO1, PBRM1, NRAS, BRCA2, NOTCH1, CREBBP, and RBX1 has been reduced, and wherein said mammalian has increased productivity for heterologous polypeptides compared to a cell that has the identical genotype but the respective endogenous gene expression of said at least one endogenous gene selected from the group of genes consisting of MYC, STK11, SMAD4, PPP2CB, RBM38, NF1, CDK12, SIN3A, PARP-1, ATM, Hipk2, BARD1, HIF1AN, SMAD3, PALB2, FUBP1, RBL2, RPS6KA3, GPS2, ETS1, E2F5, CDKN1A, RNF43, EEF2K, AKT1, BRCA1, ATR, SMAD2, BAD, FOXO1, PBRM1, NRAS, BRCA2, NOTCH1, CREBBP, and RBX1 and that is cultivated under the same conditions. In certain preferred embodiments, the endogenous gene is the MYC gene.

One independent aspect of the current invention is a method for increasing heterologous polypeptide titer of a recombinant mammalian cell, which has reduced expression of at least one endogenous gene selected from the group of genes consisting of MYC, STK11, SMAD4, PPP2CB, RBM38, NF1, CDK12, SIN3A, PARP-1, ATM, Hipk2, BARD1, HIF1AN, SMAD3, PALB2, FUBP1, RBL2, RPS6KA3, GPS2, ETS1, E2F5, CDKN1A, RNF43, EEF2K, AKT1, BRCA1, ATR, SMAD2, BAD, FOXO1, PBRM1, NRAS, BRCA2, NOTCH1, CREBBP, and RBX1, and which comprises an exogenous nucleic acid, i.e. a transgene, encoding said heterologous polypeptide compared to a cell cultivated under the same conditions that has the identical genotype but endogenous gene expression of said at least one endogenous gene selected from the group of genes consisting of MYC, STK11, SMAD4, PPP2CB, RBM38, NF1, CDK12, SIN3A, PARP-1, ATM, Hipk2, BARD1, HIF1AN, SMAD3, PALB2, FUBP1, RBL2, RPS6KA3, GPS2, ETS1, E2F5, CDKN1A, RNF43, EEF2K, AKT1, BRCA1, ATR, SMAD2, BAD, FOXO1, PBRM1, NRAS, BRCA2, NOTCH1, CREBBP, and RBX1. In certain preferred embodiments, the endogenous gene is the MYC gene.

One independent aspect of the current invention is a method for producing a recombinant mammalian cell with improved recombinant productivity, wherein the method comprises the following steps:
a) applying a nuclease-assisted and/or nucleic acid targeting at least one endogenous gene selected from the group of genes consisting of MYC, STK11, SMAD4, PPP2CB, RBM38, NF1, CDK12, SIN3A, PARP-1, ATM, Hipk2, BARD1, HIF1AN, SMAD3, PALB2, FUBP1, RBL2, RPS6KA3, GPS2, ETS1, E2F5, CDKN1A, RNF43, EEF2K, AKT1, BRCA1, ATR, SMAD2, BAD, FOXO1, PBRM1, NRAS, BRCA2, NOTCH1, CREBBP, and RBX1 in a mammalian cell to reduce the activity of said endogenous gene, and
b) selecting a mammalian cell wherein the activity of said endogenous gene has been reduced,
   thereby producing a recombinant mammalian cell with increased recombinant productivity compared to a cell cultivated under the same conditions that has the identical genotype but endogenous gene expression of said at least one endogenous gene selected from the group of genes consisting of MYC, STK11, SMAD4, PPP2CB, RBM38, NF1, CDK12, SIN3A, PARP-1, ATM, Hipk2, BARD1, HIF1AN, SMAD3, PALB2, FUBP1, RBL2, RPS6KA3, GPS2, ETS1, E2F5, CDKN1A, RNF43, EEF2K, AKT1, BRCA1, ATR, SMAD2, BAD, FOXO1, PBRM1, NRAS, BRCA2, NOTCH1, CREBBP, and RBX1. In certain preferred embodiments, the endogenous gene is the MYC gene.

One independent aspect of to the current invention is a method for producing a heterologous polypeptide comprising the steps of
a) cultivating a recombinant mammalian cell comprising an exogenous deoxyribonucleic acid encoding the heterologous polypeptide optionally under conditions suitable for the expression of the heterologous polypeptide, and
b) recovering the heterologous polypeptide from the cell or the cultivation medium,
wherein the activity or/and function or/and expression of at least one endogenous gene selected from the group of genes consisting of MYC, STK11, SMAD4, PPP2CB, RBM38, NF1, CDK12, SIN3A, PARP-1, ATM, Hipk2, BARD1, HIF1AN, SMAD3, PALB2, FUBP1, RBL2, RPS6KA3, GPS2, ETS1, E2F5, CDKN1A, RNF43, EEF2K, AKT1, BRCA1, ATR, SMAD2, BAD, FOXO1, PBRM1, NRAS, BRCA2, NOTCH1, CREBBP, and RBX1 has been reduced or eliminated or diminished or (completely) knocked-out in said mammalian cell. In certain preferred embodiments, the endogenous gene is the MYC gene.

Another independent aspect of the current invention is a method for producing a recombinant mammalian cell having/with improved and/or increased recombinant productivity, wherein the method comprises the following steps:
a) applying a nucleic acid or an enzyme or a nuclease-assisted gene targeting system targeting at least one endogenous gene selected from the group of genes consisting of MYC, STK11, SMAD4, PPP2CB, RBM38, NF1, CDK12, SIN3A, PARP-1, ATM, Hipk2, BARD1, HIF1AN, SMAD3, PALB2, FUBP1, RBL2, RPS6KA3, GPS2, ETS1, E2F5, CDKN1A, RNF43, EEF2K, AKT1, BRCA1, ATR, SMAD2, BAD, FOXO1, PBRM1, NRAS, BRCA2, NOTCH1, CREBBP, and RBX1 to a mammalian cell to reduce or eliminate or diminish or (completely) knockout the activity or/and function or/and expression of said endogenous gene, and
b) selecting a mammalian cell wherein the activity or/and function or/and expression of said endogenous gene has been reduced or eliminated or diminished or (completely) knocked-out,
thereby producing a recombinant mammalian cell having/with improved and/or increased recombinant productivity.

In certain preferred embodiments of all aspects and embodiments of the invention, the endogenous gene is the MYC gene.

In certain dependent embodiments of all aspects and embodiments of the current invention, the mammalian cell comprises a nucleic acid encoding a heterologous polypeptide.

In certain dependent embodiments of all aspects and embodiments of the current invention, the nucleic acid encoding the heterologous polypeptide is operably linked to a promoter sequence functional is said mammalian cell and operably linked to a polyadenylation signal functional in said mammalian cell. In certain embodiments, the mammalian cell secretes the heterologous polypeptide when cultivated under suitable cultivation conditions.

In certain dependent embodiments of all aspects and embodiments of the current invention, the knockout of at least one endogenous gene selected from the group of genes consisting of MYC, STK11, SMAD4, PPP2CB, RBM38, NF1, CDK12, SIN3A, PARP-1, ATM, Hipk2, BARD1, HIF1AN, SMAD3, PALB2, FUBP1, RBL2, RPS6KA3, GPS2, ETS1, E2F5, CDKN1A, RNF43, EEF2K, AKT1, BRCA1, ATR, SMAD2, BAD, FOXO1, PBRM1, NRAS, BRCA2, NOTCH1, CREBBP, and RBX1 is a heterozygous knockout or a homozygous knockout.

In certain dependent embodiments of all aspects and embodiments of the current invention, the productivity of the knockout cell line is at least 5%, preferably 10% or more, most preferred 20% or more increased compared to the respective mammalian cell with the same genotype but fully functional expression of said at least one endogenous gene selected from the group of genes consisting of MYC, STK11, SMAD4, PPP2CB, RBM38, NF1, CDK12, SIN3A, PARP-1, ATM, Hipk2, BARD1, HIF1AN, SMAD3, PALB2, FUBP1, RBL2, RPS6KA3, GPS2, ETS1, E2F5, CDKN1A, RNF43, EEF2K, AKT1, BRCA1, ATR, SMAD2, BAD, FOXO1, PBRM1, NRAS, BRCA2, NOTCH1, CREBBP, and RBX1.

In certain dependent embodiments of all aspects and embodiments of the current invention, the reduction or elimination or diminishment or knockout of at least one endogenous gene selected from the group of genes consisting of MYC, STK11, SMAD4, PPP2CB, RBM38, NF1, CDK12, SIN3A, PARP-1, ATM, Hipk2, BARD1, HIF1AN, SMAD3, PALB2, FUBP1, RBL2, RPS6KA3, GPS2, ETS1, E2F5, CDKN1A, RNF43, EEF2K, AKT1, BRCA1, ATR, SMAD2, BAD, FOXO1, PBRM1, NRAS, BRCA2, NOTCH1, CREBBP, and RBX1 is mediated by a nuclease-assisted gene targeting system. In certain embodiments, the nuclease-assisted gene targeting system is selected from the group consisting of CRISPR/Cas9, CRISPR/Cpf1, zinc-finger nuclease, TALEN and meganucleases.

In certain dependent embodiments of all aspects and embodiments of the current invention, the reduction of the expression of at least one endogenous gene selected from the group of genes consisting of MYC, STK11, SMAD4, PPP2CB, RBM38, NF1, CDK12, SIN3A, PARP-1, ATM, Hipk2, BARD1, HIF1AN, SMAD3, PALB2, FUBP1, RBL2, RPS6KA3, GPS2, ETS1, E2F5, CDKN1A, RNF43, EEF2K, AKT1, BRCA1, ATR, SMAD2, BAD, FOXO1, PBRM1, NRAS, BRCA2, NOTCH1, CREBBP, and RBX1 is mediated by RNA silencing. In certain embodiments, the RNA silencing is selected from the group consisting of siRNA gene targeting and knockdown, shRNA gene targeting and knockdown, and miRNA gene targeting and knockdown.

In certain dependent embodiments of all aspects and embodiments of the current invention, the knockout of at least one endogenous gene selected from the group of genes consisting of MYC, STK11, SMAD4, PPP2CB, RBM38, NF1, CDK12, SIN3A, PARP-1, ATM, Hipk2, BARD1, HIF1AN, SMAD3, PALB2, FUBP1, RBL2, RPS6KA3, GPS2, ETS1, E2F5, CDKN1A, RNF43, EEF2K, AKT1, BRCA1, ATR, SMAD2, BAD, FOXO1, PBRM1, NRAS, BRCA2, NOTCH1, CREBBP, and RBX1 is performed i) before the introduction of the exogenous nucleic acid encoding the heterologous polypeptide, or ii) after the introduction of the exogenous nucleic acid encoding the heterologous polypeptide.

In certain dependent embodiments of all aspects and embodiments of the current invention, the heterologous polypeptide is an antibody. In certain embodiments, the antibody is an antibody comprising two or more different binding sites and optionally a domain exchange. In certain embodiments, the antibody comprises three or more binding sites or VH/VL-pairs or Fab fragments and optionally a domain exchange. In certain embodiments, the antibody is a multispecific antibody.

In certain dependent embodiments of all aspects and embodiments of the current invention, the at least one endogenous gene is selected from the group of genes consisting of MYC, STK11, SMAD4, PPP2CB, RBM38, CDK12, PARP-1, ATM, Hipk2, BARD1, and SMAD3. In certain embodiments, the at least one endogenous gene is selected from the group of genes consisting of MYC, STK11, SMAD4, PPP2CB, RBM38, and CDK12. In certain embodiments, the at least one endogenous gene is selected from the group of genes consisting of MYC and STK11. In one preferred embodiment, the at least one endogenous gene is MYC.

In certain dependent embodiment of all aspects and embodiments of the current invention, in the recombinant mammalian cell the activity or function or expression of the endogenous SIRT-1 gene and one or more, i.e. at least one, further endogenous gene(s) selected from the group consisting of MYC, STK11, SMAD4, PPP2CB, RBM38, CDK12, PARP-1, ATM, Hipk2, BARD1, and SMAD3 has been reduced or eliminated or diminished or (completely) knocked-out. In certain embodiments, the at least one further endogenous gene is selected from the group of genes consisting of MYC, STK11, SMAD4, PPP2CB, RBM38, and CDK12. In certain embodiments, the at least one further endogenous gene is selected from the group of genes consisting of MYC and STK11. In one preferred embodiment, the at least one further endogenous gene is MYC, i.e. the activity or function or expression of the endogenous SIRT-1 and the endogenous MYC gene have been reduced or eliminated or diminished or (completely) knocked-out.

In certain dependent embodiments of all aspects and embodiments of the current invention, the heterologous polypeptide is selected from the group of heterologous polypeptides comprising multispecific antibodies and antibody-multimer-fusion polypeptide. In certain embodiments, the heterologous polypeptide is selected from the group consisting of
i) a full-length antibody with domain exchange comprising a first Fab fragment and a second Fab fragment,
   wherein in the first Fab fragment
      a) the light chain of the first Fab fragment comprises a VL and a CH1 domain and the heavy chain of the first Fab fragment comprises a VH and a CL domain;
      b) the light chain of the first Fab fragment comprises a VH and a CL domain and the heavy chain of the first Fab fragment comprises a VL and a CH1 domain; or
      c) the light chain of the first Fab fragment comprises a VH and a CH1 domain and the heavy chain of the first Fab fragment comprises a VL and a CL domain;
   and
   wherein the second Fab fragment comprises a light chain comprising a VL and a CL domain, and a heavy chain comprising a VH and a CH1 domain;
ii) a full-length antibody with domain exchange and additional heavy chain C-terminal binding site comprising
   one full length antibody comprising two pairs each of a full length antibody light chain and a full length antibody heavy chain, wherein the binding sites formed by each of the pairs of the full length heavy chain and the full length light chain specifically bind to a first antigen;
   and
      one additional Fab fragment, wherein the additional Fab fragment is fused to the C-terminus of one heavy chain of the full length antibody, wherein the binding site of the additional Fab fragment specifically binds to a second antigen;
   wherein the additional Fab fragment specifically binding to the second antigen i) comprises a domain crossover such that a) the light chain variable domain (VL) and the heavy chain variable domain (VH) are replaced by each other, or b) the light chain constant domain (CL) and the heavy chain constant domain (CH1) are replaced by each other, or ii) is a single chain Fab fragment;
iii) a one-armed single chain antibody comprising a first binding site that specifically binds to a first epitope or antigen and a second binding site that specifically binds to a second epitope or antigen, comprising
   a light chain comprising a variable light chain domain and a light chain kappa or lambda constant domain;
   a combined light/heavy chain comprising a variable light chain domain, a light chain constant domain, a peptidic linker, a variable heavy chain domain, a CH1 domain, a Hinge region, a CH2 domain, and a CH3 with knob mutation;
   a heavy chain comprising a variable heavy chain domain, a CH1 domain, a hinge region, a CH2 domain, and a CH3 domain with hole mutation;
iv) a two-armed single chain antibody comprising a first binding site that specifically binds to a first epitope or antigen and a second binding site that specifically binds to a second epitope or antigen, comprising
   a first combined light/heavy chain comprising a variable light chain domain, a light chain constant domain, a peptidic linker, a variable heavy chain domain, a CH1 domain, a Hinge region, a CH2 domain, and a CH3 with hole mutation;
   a second combined light/heavy chain comprising a variable light chain domain, a light chain constant domain, a peptidic linker, a variable heavy chain domain, a CH1 domain, a Hinge region, a CH2 domain, and a CH3 domain with knob mutation;
v) a common light chain bispecific antibody comprising a first binding site that specifically binds to a first epitope or antigen and a second binding site that specifically binds to a second epitope or antigen, comprising
   a light chain comprising a variable light chain domain and a light chain constant domain;
   a first heavy chain comprising a variable heavy chain domain, a CH1 domain, a Hinge region, a CH2 domain, and a CH3 domain with hole mutation;
   a second heavy chain comprising a variable heavy chain domain, a CH1 domain, a Hinge region, a CH2 domain, and a CH3 domain with knob mutation;
vi) a full-length antibody with additional heavy chain N-terminal binding site with domain exchange comprising
   a first and a second Fab fragment, wherein each binding site of the first and the second Fab fragment specifically bind to a first antigen;
   a third Fab fragment, wherein the binding site of the third Fab fragment specifically binds to a second antigen, and wherein the third Fab fragment comprises a domain crossover such that the variable light chain domain (VL) and the variable heavy chain domain (VH) are replaced by each other; and
   an Fc-region comprising a first Fc-region polypeptide and a second Fc-region polypeptide;
   wherein the first and the second Fab fragment each comprise a heavy chain fragment and a full-length light chain, wherein the C-terminus of the heavy chain fragment of the first Fab fragment is fused to the N-terminus of the first Fc-region polypeptide, wherein the C-terminus of the heavy chain fragment of the second Fab fragment is fused to the N-terminus of the variable light chain domain of the third Fab fragment and the C-terminus of the CH1 domain of the third Fab fragment is fused to the N-terminus of the second Fc-region polypeptide;
   vii) an immunoconjugate comprising a full-length antibody and a non-immunoglobulin moiety conjugated to each other optionally via a peptidic linker,
and
viii) an antibody-multimer-fusion polypeptide comprising
   (a) an antibody heavy chain and an antibody light chain, and
   (b) a first fusion polypeptide comprising in N- to C-terminal direction a first part of a non-antibody multimeric polypeptide, an antibody heavy chain CH1 domain or an antibody light chain constant domain, an antibody hinge region, an antibody heavy chain CH2 domain and an antibody heavy chain CH3 domain, and a second fusion polypeptide comprising in N- to C-terminal direction the second part of the non-antibody multimeric polypeptide and an antibody light chain constant domain if the first polypeptide comprises an antibody heavy chain CH1 domain or an antibody heavy chain CH1 domain if the first polypeptide comprises an antibody light chain constant domain,
   wherein
      (i) the antibody heavy chain of (a) and the first fusion polypeptide of (b), (ii) the antibody heavy chain of (a) and the antibody light chain of (a), and (iii) the first fusion polypeptide of (b) and the second fusion polypeptide of (b) are each independently of each other covalently linked to each other by at least one disulfide bond,
   wherein
      the variable domains of the antibody heavy chain and the antibody light chain form a binding site specifically binding to an antigen.

In certain preferred embodiments, the at least one endogenous gene is selected from the group of genes consisting of MYC, STK11, SMAD4, PPP2CB, RBM38, CDK12, PARP-1, ATM, Hipk2, BARD1, and SMAD3. In certain embodiments, the at least one endogenous gene is selected from the group of genes consisting of MYC, STK11, SMAD4, PPP2CB, RBM38, and CDK12. In certain embodiments, the at least one endogenous gene is selected from the group of genes consisting of MYC and STK11. In one preferred embodiment, the at least one endogenous gene is MYC. In one further preferred embodiment, further the activity or function or expression of the endogenous SIRT-1 gene has been reduced or eliminated or diminished or (completely) knocked-out.

In certain embodiments, in the recombinant mammalian cell the activity or function or expression of the endogenous SIRT-1 gene and one or more, i.e. at least one, further endogenous gene(s) selected from the group consisting of MYC, STK11, SMAD4, PPP2CB, RBM38, CDK12, PARP-1, ATM, Hipk2, BARD1, and SMAD3 has been reduced or eliminated or diminished or (completely) knocked-out. In certain embodiments, the at least one further endogenous gene is selected from the group of genes consisting of MYC, STK11, SMAD4, PPP2CB, RBM38, and CDK12. In certain embodiments, the at least one further endogenous gene is selected from the group of genes consisting of MYC and STK11. In one preferred embodiment, the at least one further endogenous gene is MYC, i.e. the activity or function or expression of the endogenous SIRT-1 gene and the endogenous MYC gene has been reduced or eliminated or diminished or (completely) knocked-out.

In certain dependent embodiments of all aspects and embodiments of the current invention, the at least one endogenous gene is selected from the group of genes consisting of MYC, STK11, SMAD4, PPP2CB, RBM38, NF1, CDK12, SIN3A, PARP-1, ATM, Hipk2, BARD1, HIF1AN, SMAD3, and CDKN1A. In certain embodiments, the at least one endogenous gene is selected from the group of genes consisting of MYC, STK11, SMAD4, PPP2CB, RBM38, NF1, and CDK12. In certain embodiments, the at least one endogenous gene is selected from the group of genes consisting of MYC, STK11, SMAD4, and PPP2CB. In one preferred embodiment, the at least one endogenous gene is MYC.

In certain dependent embodiments of all aspects and embodiments of the current invention, in the recombinant mammalian cell the activity or function or expression of the endogenous SIRT-1 gene and one or more, i.e. at least one, further endogenous gene(s) selected from the group consisting of MYC, STK11, SMAD4, PPP2CB, RBM38, NF1, CDK12, SIN3A, PARP-1, ATM, Hipk2, BARD1, HIF1AN, SMAD3, and CDKN1A has been reduced or eliminated or diminished or (completely) knocked-out. In certain embodiments, the at least one further endogenous gene is selected from the group of genes consisting of MYC, STK11, SMAD4, PPP2CB, RBM38, NF1, and CDK12. In certain embodiments, the at least one further endogenous gene is selected from the group of genes consisting of MYC, STK11, SMAD4, and PPP2CB. In one preferred embodiment, the activity or function or expression of the endogenous SIRT-1 gene and of the endogenous MYC gene has been reduced or eliminated or diminished or (completely) knocked out.

In certain dependent embodiments of all aspects and embodiments of the current invention, the heterologous polypeptide is an antibody-multimer-fusion polypeptide comprising
(a) an antibody heavy chain and an antibody light chain, and
(b) a first fusion polypeptide comprising in N- to C-terminal direction a first part of a non-antibody multimeric polypeptide, an antibody heavy chain CH1 domain or an antibody light chain constant domain, an antibody hinge region, an antibody heavy chain CH2 domain and an antibody heavy chain CH3 domain, and a second fusion polypeptide comprising in N- to C-terminal direction the second part of the non-antibody multimeric polypeptide and an antibody light chain constant domain if the first polypeptide comprises an antibody heavy chain CH1 domain or an antibody heavy chain CH1 domain if the first polypeptide comprises an antibody light chain constant domain,
wherein
   (i) the antibody heavy chain of (a) and the first fusion polypeptide of (b), (ii) the antibody heavy chain of (a) and the antibody light chain of (a), and (iii) the first fusion polypeptide of (b) and the second fusion polypeptide of (b) are each independently of each other covalently linked to each other by at least one disulfide bond,
wherein
   the variable domains of the antibody heavy chain and the antibody light chain form a binding site specifically binding to an antigen.

In certain embodiments, the at least one endogenous gene is selected from the group of genes consisting of MYC, STK11, SMAD4, PPP2CB, RBM38, NF1, CDK12, SIN3A, PARP-1, ATM, Hipk2, BARD1, HIF1AN, SMAD3, and CDKN1A. In certain embodiments, the at least one endogenous gene is selected from the group of genes consisting of MYC, STK11, SMAD4, PPP2CB, RBM38, NF1, and CDK12. In certain embodiments, the at least one endogenous gene is selected from the group of genes consisting of MYC, STK11, SMAD4, and PPP2CB. In one preferred embodiment, the at least one endogenous gene is MYC. In one further preferred embodiment, in addition the activity or function or expression of the endogenous SIRT-1 gene has been reduced or eliminated or diminished or (completely) knocked-out.

In certain embodiments, in the recombinant mammalian cell the activity or function or expression of the endogenous SIRT-1 gene and one or more, i.e. at least one, further endogenous gene(s) selected from the group consisting of MYC, STK11, SMAD4, PPP2CB, RBM38, NF1, CDK12, SIN3A, PARP-1, ATM, Hipk2, BARD1, HIF1AN, SMAD3, and CDKN1A has been reduced or eliminated or diminished or (completely) knocked-out. In certain embodiments, the at least one further endogenous gene is selected from the group of genes consisting of MYC, STK11, SMAD4, PPP2CB, RBM38, NF1, and CDK12. In certain embodiments, the at least one further endogenous gene is selected from the group of genes consisting of MYC, STK11, SMAD4, and PPP2CB. In one preferred embodiment, the at least one further endogenous gene is MYC.

In certain dependent embodiments, the first fusion polypeptide comprises as first part of the non-antibody multimeric polypeptide two ectodomains of a TNF ligand family member or a fragment thereof that are connected to each other by a peptide linker, and the second fusion polypeptide comprises as second part of a non-antibody multimeric polypeptide only one ectodomain of said TNF ligand family member or a fragment thereof, or vice versa. In certain embodiments, the first fusion polypeptide comprises in N- to C-terminal direction a first part of a non-antibody multimeric polypeptide, an antibody light chain constant domain, an antibody hinge region, an antibody heavy chain CH2 domain and an antibody heavy chain CH3 domain, and the second fusion polypeptide comprising in N- to C-terminal direction the second part of the non-antibody multimeric polypeptide and an antibody heavy chain CH1 domain. In certain embodiments, in the CL domain adjacent to the part of a non-antibody multimeric polypeptide the amino acid at position 123 (Kabat EU numbering) has been replaced by arginine (R) and the amino acid at position 124 (Kabat EU numbering) has been substituted by lysine (K), and wherein in the CH1 domain adjacent to the part of a non-antibody multimeric polypeptide the amino acids at position 147 (Kabat EU numbering) and at position 213 (Kabat EU numbering) have been substituted by glutamic acid (E). In certain embodiments, the variable domains of the antibody heavy chain and the antibody light chain form a binding site specifically binding to a cell surface antigen selected from the group consisting of Fibroblast Activation Protein (FAP), Melanoma-associated Chondroitin Sulfate Proteoglycan (MCSP), Epidermal Growth Factor Receptor (EGFR), Carcinoembryonic Antigen (CEA), CD19, CD20 and CD33. In certain embodiments, TNF ligand family member co-stimulates human T-cell activation. In certain embodiments, the TNF ligand family member is selected from 4-1BBL and OX40L. In one preferred embodiment, the TNF ligand family member is 4-1BBL and the cell surface antigen is FAP or CD19 or CEA.

In certain dependent embodiments of all aspects and embodiments of the current invention, the heterologous polypeptide is a fusion polypeptide comprising a bivalent, mono- or bispecific full-length antibody and a non-immunoglobulin moiety, wherein the antibody is conjugated to the non-immunoglobulin moiety at a single terminus of one of the heavy or light chains of the antibody optionally via a peptidic linker. In certain embodiments, the at least one endogenous gene is selected from the group of genes consisting of MYC, STK11, SMAD4, PPP2CB, RBM38, CDK12, PARP-1, ATM, Hipk2, BARD1, SMAD3, PALB2, FUBP1, RBL2, RPS6KA3, GPS2, ETS1, E2F5, CDKN1A, RNF43, EEF2K, AKT1, BRCA1, ATR, SMAD2, BAD, FOXO1, PBRM1, NRAS, BRCA2, NOTCH1, CREBBP, and RBX1. In certain embodiments, the at least one endogenous gene is selected from the group of genes consisting of MYC, STK11, SMAD4, PPP2CB, RBM38, CDK12, PARP-1, BARD1, ETS1, E2F5, RNF43, EEF2K, AKT1, BRCA1, BAD, FOXO1, PBRM1, BRCA2, NOTCH1, and CREBBP. In certain embodiments, the at least one endogenous gene is selected from the group of genes consisting of MYC, STK11, and CDK12. In one preferred embodiment, the at least one endogenous gene is MYC. In one further preferred embodiment, in addition the activity or function or expression of the endogenous SIRT-1 gene has been reduced or eliminated or diminished or (completely) knocked-out. In certain embodiments, the heterologous polypeptide is an anti-PD-1 antibody conjugated to interleukin-2. In certain embodiments, the interleukin-2 is an engineered IL2v moiety with abolished binding to IL-2Ra (CD25) to avoid undesired CD25-mediated toxicities and Treg expansion.

In certain dependent embodiment of all aspects and embodiments of the invention, the mammalian cell is a CHO cell or a HEK cell. In one preferred embodiment, the mammalian cell is a CHO-K1 cell. In certain embodiments, the mammalian cell is a suspension growing mammalian cell.

In certain dependent embodiments of all aspects and embodiments of the invention, the productivity for heterologous polypeptides is determined in a 4-day batch cultivation. In certain embodiments, the 4-day batch cultivation is inoculated/started with a cell density of at least 1*10⁶ cells/ml (10E6 cells/ml). In certain embodiments, the 4-day batch cultivation is inoculated/started with a cell density of at least 2*10⁶ cells/ml. In certain embodiments, the 4-day batch cultivation is inoculated/started with a cell density of at least 5*10⁶ cells/ml. In certain embodiments, the 4-day batch cultivation is inoculated/started with a cell density of at least 10*10⁶ cells/ml. In certain embodiments, the 4-day batch cultivation is in a chemically defined, serum-free medium.

In certain dependent embodiments of all aspects and embodiments of the invention, the reduction or elimination or diminishment or knockout of the one, two or more endogenous genes is by a CRISPR/Cas9 nuclease-assisted gene targeting system. In one preferred embodiment, the endogenous gene is SIRT-1 and three guide RNAs of SEQ ID NO: 12, 13 and 14 are used. In one further preferred embodiment, the endogenous gene is MYC and three guide RNAs of SEQ ID NO: 15, 16 and 17 are used. In certain embodiments, the endogenous gene is STK11 and three guide RNAs of SEQ ID NO: 18, 19 and 20 are used. In certain embodiments, the endogenous gene is SMAD4 and three guide RNAs of SEQ ID NO: 21, 22 and 23 are used. In certain embodiments, the endogenous gene is PPP2CB and three guide RNAs of SEQ ID NO: 24, 25 and 26 are used. In certain embodiments, the endogenous gene is RBM38 and three guide RNAs of SEQ ID NO: 27, 28 and 29 are used. In certain embodiments, the endogenous gene is NF1 and three guide RNAs of SEQ ID NO: 30, 31 and 32 are used. In certain embodiments, the endogenous gene is CDK12 and three guide RNAs of SEQ ID NO: 33, 34 and 35 are used. In certain embodiments, the endogenous gene is SIN3A and three guide RNAs of SEQ ID NO: 36, 37 and 38 are used. In certain embodiments, the endogenous gene is PARP-1 and three guide RNAs of SEQ ID NO: 39, 40 and 41 are used. In certain embodiments, the endogenous gene is ATM and three guide RNAs of SEQ ID NO: 42, 43 and 44 are used. In certain embodiments, the endogenous gene is Hipk2 and three guide RNAs of SEQ ID NO: 45, 46 and 47 are used. In certain embodiments, the endogenous gene is BARD1 and three guide RNAs of SEQ ID NO: 48, 49 and 50 are used. In certain embodiments, the endogenous gene is HIF1AN and three guide RNAs of SEQ ID NO: 51, 52 and 53 are used. In certain embodiments, the endogenous gene is SMAD3 and three guide RNAs of SEQ ID NO: 54, 55 and 56 are used. In certain embodiments, the endogenous gene is CDKN1A and three guide RNAs of SEQ ID NO: 57, 58 and 59 are used.

In addition to the various embodiments depicted and claimed, the disclosed subject matter is also directed to other embodiments having other combinations of the features disclosed and claimed herein. As such, the particular features presented herein can be combined with each other in other manners within the scope of the disclosed subject matter such that the disclosed subject matter includes any suitable combination of the features disclosed herein. The description of specific embodiments of the disclosed subject matter has been presented for purposes of illustration and description. It is not intended to be exhaustive or to limit the disclosed subject matter to those embodiments disclosed.

### DETAILED DESCRIPTION OF EMBODIMENTS OF THE INVENTION

Herein is reported a method for generating a recombinant mammalian cell expressing a heterologous polypeptide and a method for producing a heterologous polypeptide using said recombinant mammalian cell, wherein in the recombinant cell the activity/function/expression of at least one endogenous gene selected from the group of genes consisting of MYC, STK11, SMAD4, PPP2CB, RBM38, NF1, CDK12, SIN3A, PARP-1, ATM, Hipk2, BARD1, HIF1AN, SMAD3, PALB2, FUBP1, RBL2, RPS6KA3, GPS2, ETS1, E2F5, CDKN1A, RNF43, EEF2K, AKT1, BRCA1, ATR, SMAD2, BAD, FOXO1, PBRM1, NRAS, BRCA2, NOTCH1, CREBBP, and RBX1 has been reduced/eliminated/diminished/(completely) knocked-out.

The invention is based, at least in part, on the finding that the knockout of at least one endogenous gene selected from the group of genes consisting of MYC, STK11, SMAD4, PPP2CB, RBM38, NF1, CDK12, SIN3A, PARP-1, ATM, Hipk2, BARD1, HIF1AN, SMAD3, PALB2, FUBP1, RBL2, RPS6KA3, GPS2, ETS1, E2F5, CDKN1A, RNF43, EEF2K, AKT1, BRCA1, ATR, SMAD2, BAD, FOXO1, PBRM1, NRAS, BRCA2, NOTCH1, CREBBP, and RBX1 in a mammalian cell, e.g. such as a CHO cell, improves recombinant productivity, e.g. of standard IgG-type antibodies and especially of complex antibody formats.

### I. General Definitions

Useful methods and techniques for carrying out the current invention are described in e.g. Ausubel, F. M. (ed.), Current Protocols in Molecular Biology, Volumes I to III (1997); Glover, N. D., and Hames, B. D., ed., DNA Cloning: A Practical Approach, Volumes I and II (1985), Oxford University Press; Freshney, R. I. (ed.), Animal Cell Culture—a practical approach, IRL Press Limited (1986); Watson, J. D., et al., Recombinant DNA, Second Edition, CHSL Press (1992); Winnacker, E. L., From Genes to Clones; N.Y., VCH Publishers (1987); Celis, J., ed., Cell Biology, Second Edition, Academic Press (1998); Freshney, R. I., Culture of Animal Cells: A Manual of Basic Technique, second edition, Alan R. Liss, Inc., N.Y. (1987).

The use of recombinant DNA technology enables the generation of derivatives of a nucleic acid. Such derivatives can, for example, be modified in individual or several nucleotide positions by substitution, alteration, exchange, deletion or insertion. The modification or derivatization can, for example, be carried out by means of site directed mutagenesis. Such modifications can easily be carried out by a person skilled in the art (see e.g. Sambrook, J., et al., Molecular Cloning: A laboratory manual (1999) Cold Spring Harbor Laboratory Press, New York, USA; Hames, B. D., and Higgins, S. G., Nucleic acid hybridization—a practical approach (1985) IRL Press, Oxford, England).

It must be noted that as used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art, and so forth. As well, the terms “a” (or “an”), “one or more” and “at least one” can be used interchangeably herein. It is also to be noted that the terms “comprising”, “including”, and “having” can be used interchangeably.

The term “about” denotes a range of +/−20% of the thereafter following numerical value. In certain embodiments, the term about denotes a range of +/−10% of the thereafter following numerical value. In certain embodiments, the term about denotes a range of +/−5% of the thereafter following numerical value.

The term “comprising” also encompasses the term “consisting of”.

The term “recombinant mammalian cell” as used herein denotes a mammalian cell comprising an exogenous nucleotide sequence capable of expressing a polypeptide. Such recombinant mammalian cells are cells into which one or more exogenous nucleic acid(s) have been introduced, including the progeny of such cells. Thus, the term “a mammalian cell comprising a nucleic acid encoding a heterologous polypeptide” denotes cells comprising an exogenous nucleotide sequence integrated in the genome of the mammalian cell and capable of expressing the heterologous polypeptide. In certain embodiments, the mammalian cell comprising an exogenous nucleotide sequence is a cell comprising an exogenous nucleotide sequence integrated at a single site within a locus of the genome of the host cell, wherein the exogenous nucleotide sequence comprises a first and a second recombination recognition sequence flanking at least one first selection marker, and a third recombination recognition sequence located between the first and the second recombination recognition sequence, and all the recombination recognition sequences are different

The term “recombinant cell” as used herein denotes a cell after genetic modification, such as, e.g., a cell expressing a heterologous polypeptide of interest and that can be used for the production of said heterologous polypeptide of interest at any scale. For example, “a recombinant mammalian cell comprising an exogenous nucleotide sequence” denotes a cell wherein the coding sequences for a heterologous polypeptide of interest have been introduced into the genome of the host cell. For example, “a recombinant mammalian cell comprising an exogenous nucleotide sequence” that has been subjected to recombinase mediated cassette exchange (RMCE) whereby the coding sequences for a polypeptide of interest have been introduced into the genome of the host cell is a “recombinant cell”.

A “mammalian cell comprising an exogenous nucleotide sequence” and a “recombinant cell” are both “transformed cells”. This term includes the primary transformed cell as well as progeny derived therefrom without regard to the number of passages. Progeny may, e.g., not be completely identical in nucleic acid content to a parent cell, but may contain mutations. Mutant progeny that has the same function or biological activity as screened or selected for in the originally transformed cell are encompassed.

An “isolated” composition is one, which has been separated from a component of its natural environment. In some embodiments, a composition is purified to greater than 95% or 99% purity as determined by, for example, electrophoretic (e.g., SDS-PAGE, isoelectric focusing (IEF), capillary electrophoresis, CE-SDS) or chromatographic (e.g., size exclusion chromatography or ion exchange or reverse phase HPLC). For review of methods for assessment of e.g. antibody purity, see, e.g., Flatman, S. et al., J. Chrom. B 848 (2007) 79-87.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

An “isolated” polypeptide or antibody refers to a polypeptide molecule or antibody molecule that has been separated from a component of its natural environment.

The term “integration site” denotes a nucleic acid sequence within a cell's genome into which an exogenous nucleotide sequence is inserted. In certain embodiments, an integration site is between two adjacent nucleotides in the cell's genome. In certain embodiments, an integration site includes a stretch of nucleotide sequences. In certain embodiments, the integration site is located within a specific locus of the genome of a mammalian cell. In certain embodiments, the integration site is within an endogenous gene of a mammalian cell.

The terms “vector” or “plasmid”, which can be used interchangeably, as used herein, refer to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”.

The term “binding to” denotes the binding of a binding site to its target, such as e.g. of an antibody binding site comprising an antibody heavy chain variable domain and an antibody light chain variable domain to the respective antigen. This binding can be determined using, for example, a BIAcore® assay (GE Healthcare, Uppsala, Sweden). That is, the term “binding (to an antigen)” denotes the binding of an antibody in an in vitro assay to its antigen(s). In certain embodiments, binding is determined in a binding assay in which the antibody is bound to a surface and binding of the antigen to the antibody is measured by Surface Plasmon Resonance (SPR). The term “binding” also includes the term “specifically binding”.

For example, in one possible embodiment of the BIAcore® assay, the antigen is bound to a surface and binding of the antibody, i.e. its binding site(s), is measured by surface plasmon resonance (SPR). The affinity of the binding is defined by the terms kₐ (association constant: rate constant for the association to form a complex), k_{d} (dissociation constant; rate constant for the dissociation of the complex), and K_{D} (k_{d}/kₐ). Alternatively, the binding signal of a SPR sensorgram can be compared directly to the response signal of a reference, with respect to the resonance signal height and the dissociation behaviors.

The term “binding site” denotes any proteinaceous entity that shows binding specificity to a target. This can be, e.g., a receptor, a receptor ligand, an anticalin, an affibody, an antibody, etc. Thus, the term “binding site” as used herein denotes a polypeptide that can specifically bind to or can be specifically bound by a second polypeptide.

As used herein, the term “selection marker” denotes a gene that allows cells carrying the gene to be specifically selected for or against, in the presence of a corresponding selection agent. For example, but not by way of limitation, a selection marker can allow the host cell transformed with the selection marker gene to be positively selected for in the presence of the respective selection agent (selective cultivation conditions); a non-transformed host cell would not be capable of growing or surviving under the selective cultivation conditions. Selection markers can be positive, negative or bi-functional. Positive selection markers can allow selection for cells carrying the marker, whereas negative selection markers can allow cells carrying the marker to be selectively eliminated. A selection marker can confer resistance to a drug or compensate for a metabolic or catabolic defect in the host cell. In prokaryotic cells, amongst others, genes conferring resistance against ampicillin, tetracycline, kanamycin or chloramphenicol can be used. Resistance genes useful as selection markers in eukaryotic cells include, but are not limited to, genes for aminoglycoside phosphotransferase (APH) (e.g., hygromycin phosphotransferase (HYG), neomycin and G418 APH), dihydrofolate reductase (DHFR), thymidine kinase (TK), glutamine synthetase (GS), asparagine synthetase, tryptophan synthetase (indole), histidinol dehydrogenase (histidinol D), and genes encoding resistance to puromycin, blasticidin, bleomycin, phleomycin, chloramphenicol, Zeocin, and mycophenolic acid. Further marker genes are described in WO 92/08796 and WO 94/28143.

Beyond facilitating a selection in the presence of a corresponding selection agent, a selection marker can alternatively be a molecule normally not present in the cell, e.g., green fluorescent protein (GFP), enhanced GFP (eGFP), synthetic GFP, yellow fluorescent protein (YFP), enhanced YFP (eYFP), cyan fluorescent protein (CFP), mPlum, mCherry, tdTomato, mStrawberry, J-red, DsRed-monomer, mOrange, mKO, mCitrine, Venus, YPet, Emerald, CyPet, mCFPm, Cerulean, and T-Sapphire. Cells expressing such a molecule can be distinguished from cells not harboring this gene, e.g., by the detection or absence, respectively, of the fluorescence emitted by the encoded polypeptide.

As used herein, the term “operably linked” refers to a juxtaposition of two or more components, wherein the components are in a relationship permitting them to function in their intended manner. For example, a promoter and/or an enhancer is operably linked to a coding sequence if the promoter and/or enhancer acts to modulate the transcription of the coding sequence. In certain embodiments, DNA sequences that are “operably linked” are contiguous and adjacent on a single chromosome. In certain embodiments, e.g., when it is necessary to join two protein encoding regions, such as a secretory leader and a polypeptide, the sequences are contiguous, adjacent, and in the same reading frame. In certain embodiments, an operably linked promoter is located upstream of the coding sequence and can be adjacent to it. In certain embodiments, e.g., with respect to enhancer sequences modulating the expression of a coding sequence, the two components can be operably linked although not adjacent. An enhancer is operably linked to a coding sequence if the enhancer increases transcription of the coding sequence. Operably linked enhancers can be located upstream, within, or downstream of coding sequences and can be located at a considerable distance from the promoter of the coding sequence. Operable linkage can be accomplished by recombinant methods known in the art, e.g., using PCR methodology and/or by ligation at convenient restriction sites. If convenient restriction sites do not exist, then synthetic oligonucleotide adaptors or linkers can be used in accord with conventional practice. An internal ribosomal entry site (IRES) is operably linked to an open reading frame (ORF) if it allows initiation of translation of the ORF at an internal location in a 5′-end-independent manner.

As used herein, the term “exogenous” indicates that a nucleotide sequence does not originate from a specific cell and is introduced into said cell by DNA delivery methods, e.g., by transfection, electroporation, or transformation methods. Thus, an exogenous nucleotide sequence is an artificial sequence wherein the artificiality can originate, e.g., from the combination of subsequences of different origin (e.g. a combination of a recombinase recognition sequence with an SV40 promoter and a coding sequence of green fluorescent protein is an artificial nucleic acid) or from the deletion of parts of a sequence (e.g. a sequence coding only the extracellular domain of a membrane-bound receptor or a cDNA) or the mutation of nucleobases. The term “endogenous” refers to a nucleotide sequence originating from a cell. An “exogenous” nucleotide sequence can have an “endogenous” counterpart that is identical in base compositions, but where the “exogenous” sequence is introduced into the cell, e.g., via recombinant DNA technology.

As used herein, the term “heterologous” indicates that a polypeptide does not originate from a specific cell and the respective encoding nucleic acid has been introduced into said cell by DNA delivery methods, e.g., by transfection, electroporation, or transformation methods. Thus, a heterologous polypeptide is a polypeptide that is artificial to the cell expressing it, whereby this is independent whether the polypeptide is a naturally occurring polypeptide originating from a different cell/organism or is a man-made polypeptide.

The following endogenous genes were used in the current patent application:

| | | Genomic Location |
|---|---|---|
| | | (PICR Genome) (see https:// |
| gene name | gene name | www.ncbi.nlm.nih.gov/ |
| (short) | (long) | assembly/GCF_003668045.3/) |
| PARP-1 | Poly [ADP-ribose] | RAZU01000210.1 |
| | polymerase 1 | (10105791 . . . 10139187) |
| TP53 | cellular tumor antigen | RAZU01001831.1 |
| | p53 | (22262700 . . . 22274638) |
| RBL2 | retinoblastoma-like | RAZU01000121.1 |
| | protein 2 | (4876495 . . . 4928147) |
| HIF1A | hypoxia-inducible factor | RAZU01000203.1 |
| | 1-alpha | (5550521 . . . 5594976) |
| ATM | Serine-protein kinase | RAZU01000166.1 |
| | ATM | (10510024 . . . 10617455) |
| ATR | serine/threonine-protein | RAZU01000152.1 |
| | kinase ATR | (2749722 . . . 2843344) |
| CHEK1 | Serine/threonine-protein | RAZU01000159.1 |
| | kinase Chk1 | (13545863 . . . 13571908) |
| CHEK2 | Serine/threonine-protein | RAZU01000171.1 |
| | kinase Chk2 | (3263946 . . . 3299427) |
| MYC | myc proto-oncogene | RAZU01000002.1 |
| | protein | (8,114,040-8,118,048) |
| MAPK14 | mitogen-activated | RAZU01000025.1 |
| | protein kinase 14 | (1847268 . . . 1905165) |
| MAPK8IP3 | C-Jun-amino-terminal | RAZU01000243.1 |
| | kinase-interacting | (1484490 . . . 1526846) |
| | protein 3 | |
| RPS6KA5 | ribosomal protein S6 | RAZU01000219.1 |
| | kinase alpha-5 | (6304345 . . . 6459504) |
| CAMK1 | Calcium/calmodulin- | RAZU01000261.1 |
| | dependent protein kinase | (6843427 . . . 6854833) |
| | type 1 | |
| MAPK3 | mitogen-activated | RAZU01000108.1 |
| | protein kinase 3 | (4173519 . . . 4179757) |
| MAPK1 | mitogen-activated | RAZU01000162.1 |
| | protein kinase 1 | (64068603 . . . 64135371) |
| MAPK7 | mitogen-activated | RAZU01001831.1 |
| | protein kinase 7 | (30198277 . . . 30203533) |
| MAPK8 | mitogen-activated | RAZU01001824.1 |
| | protein kinase 8 | (18958606 . . . 19018915) |
| MAPK9 | mitogen-activated | RAZU01001831.1 |
| | protein kinase 9 | (41343903 . . . 41377874) |
| JUN | Transcription factor AP-1 | RAZU01000092.1 |
| | | (10,321,409-10,322,413) |
| ETS1 | protein C-ets-1 | RAZU01000159.1 |
| | | (9938542 . . . 10064139) |
| CDKN1A | cyclin-dependent kinase | RAZU01000063.1 |
| | inhibitor 1 | (8736827 . . . 8768979) |
| CDKN1B | cyclin-dependent kinase | RAZU01000267.1 |
| | inhibitor 1B | (3559438 . . . 3562639) |
| RBM38 | RNA-binding protein 38 | RAZU01000236.1 |
| | | (501782 . . . 514198) |
| BRCA1 | breast cancer type 1 | RAZU01000248.1 |
| | susceptibility protein | (5492375 . . . 5559781) |
| BRCA2 | breast cancer type 2 | RAZU01000163.1 |
| | susceptibility protein | (13683906 . . . 13727345) |
| BARD1 | BRCA1 -associated | RAZU01000074.1 |
| | RING domain protein 1 | (44502103 . . . 44567572) |
| BAD | bcl2-associated agonist | RAZU01000139.1 |
| | of cell death | (1824994 . . . 1834442) |
| PALB2 | partner and localizer of | RAZU01000142.1 |
| | BRCA2 | (13674554 . . . 13704996) |
| E2F5 | transcription factor E2F5 | RAZU01000085.1 |
| | | (1090544 . . . 1099063) |
| E2F7 | transcription factor E2F7 | RAZU01000050.1 |
| | | (41619720 . . . 41658829) |
| E2F1 | transcription factor E2F1 | RAZU01000234.1 |
| | | (6435980 . . . 6446047) |
| NRAS | GTPase NRas | RAZU01000058.1 |
| | | (8061028 . . . 8071887) |
| AJUBA | LIM domain-containing | RAZU01001829.1 |
| | protein ajuba | (24004472 . . . 24014238) |
| CDKN1C | cyclin-dependent kinase | RAZU01000139.1 |
| | inhibitor 1C | (7236277 . . . 7239233) |
| CDKN2A | cyclin-dependent kinase | RAZU01000092.1 |
| | inhibitor 2A | (5259229 . . . 5282526) |
| CDKN2C | cyclin-dependent kinase | RAZU01000100.1 |
| | 4 inhibitor C | (7641948 . . . 7646765) |
| CDKN2D | cyclin-dependent kinase | RAZU01000153.1 |
| | 4 inhibitor D | (2505304 . . . 2508195) |
| CTNNB1 | catenin beta-1 | RAZU01000168.1 |
| | | (7895546 . . . 7923567) |
| RBL1 | retinoblastoma-like | RAZU01000234.1 |
| | protein 1 | (3736984 . . . 3793492) |
| CDK12 | Cyclin-dependent kinase 12 | RAZU01000239.1 |
| | | (886868 . . . 960350) |
| CREBBP | CREB-binding protein | RAZU01000238.1 |
| | | (6167611 . . . 6292537) |
| KEAP1 | kelch-like ECH- | RAZU01000153.1 |
| | associated protein 1 | (2447418 . . . 2457102) |
| RBX1 | E3 ubiquitin-protein | RAZU01000104.1 |
| | ligase RBX1 | (8749384 . . . 8760872) |
| CUL3 | cullin-3 isoform | RAZU01000074.1 |
| | | (52954049 . . . 53011488) |
| EEF2K | eukaryotic elongation | RAZU01000142.1 |
| | factor 2 kinase | (12417644 . . . 12495495) |
| EPHA2 | ephrin type-A receptor 2 | RAZU01000087.1 |
| | | (22640962 . . . 22669214) |
| FBXW7 | F-box/WD repeat- | RAZU01000055.1 |
| | containing protein 7 | (5504394 . . . 5672198) |
| FUBP1 | far upstream element- | RAZU01000070.1 |
| | binding protein 1 | (462251 . . . 490149) |
| GPS2 | G protein pathway | RAZU01001831.1 |
| | suppressor 2 | (21929545 . . . 21932898) |
| LATS1 | serine/threonine-protein | RAZU01000097.1 |
| | kinase LATS1 | (13938585 . . . 13971510) |
| LATS2 | serine/threonine-protein | RAZU01001829.1 |
| | kinase LATS2 | (20924105 . . . 20982104) |
| NF1 | Neurofibromin | RAZU01001831.1 |
| | | (12672140 . . . 12907880) |
| NF2 | Merlin | RAZU01000007.1 |
| | | (39720789 . . . 39808967) |
| NOTCH1 | neurogenic locus notch | RAZU01000235.1 |
| | homolog protein 1 | (8535996 . . . 8583615) |
| PBRM1 | protein polybromo-1 | RAZU01001824.1 |
| | | (21586990 . . . 21682617) |
| PIK3R1 | phosphatidylinositol 3- | RAZU01000096.1 |
| | kinase regulatory | (28560744 . . . 28642945) |
| | subunit alpha | |
| PTCH1 | protein patched homolog 1 | RAZU01000141.1 |
| | | (1684039 . . . 1742646) |
| RASA1 | ras GTPase-activating | RAZU01000096.1 |
| | protein 1 | (43508766 . . . 43582867) |
| RNF43 | E3 ubiquitin-protein | RAZU01001831.1 |
| | ligase RNF43 | (4789068 . . . 4803786) |
| RPS6KA3 | ribosomal protein S6 | RAZU01000317.1 |
| | kinase alpha-3 | (17062517 . . . 17172894) |
| SIN3A | Paired amphipathic helix | RAZU01000166.1 |
| | protein Sin3a | (7107379 . . . 7169085) |
| MXI1 | max-interacting protein 1 | RAZU01000135.1 |
| | | (8192230 . . . 8256417) |
| STK11 | Serine/threonine-protein | RAZU01000219.1 |
| | kinase STK11 | (10540304 . . . 10556926) |
| VHL | von Hippel-Lindau | RAZU01000261.1 |
| | disease tumor suppressor | (7126679 . . . 7136866) |
| HTATIP2 | oxidoreductase | RAZU01000145.1 |
| | HTATIP2 | (2122209 . . . 2135819) |
| NFKBIA | NF-kappa-B inhibitor | RAZU01000224.1 |
| | alpha | (8262980 . . . 8266215) |
| EIF4EBP1 | eukaryotic translation | RAZU01000071.1 |
| | initiation factor 4E- | (5134235 . . . 5148951) |
| | binding protein 1 | |
| NUPR1 | nuclear protein 1 | RAZU01000108.1 |
| | | (4354383 . . . 4356447) |
| FOXO3 | forkhead box protein O3 | RAZU01000099.1 |
| | | (11402271 . . . 11503619) |
| FOXO1 | forkhead box protein O1 | RAZU01000067.1 |
| | | (2653847 . . . 2733409) |
| SMAD2 | mothers against | RAZU01000075.1 |
| | decapentaplegic | (3236186 . . . 3304015) |
| | homolog 2 | |
| SMAD3 | mothers against | RAZU01000166.1 |
| | decapentaplegic homolog 3 | (480665 . . . 597614) |
| SMAD4 | mothers against | RAZU01000075.1 |
| | decapentaplegic homolog 4 | (702683 . . . 754185) |
| HIF1AN | hypoxia-inducible factor | RAZU01000135.1 |
| | 1-alpha inhibitor | (17000935 . . . 17015724) |
| EGLN2 | egl nine homolog 2 | RAZU01000274.1 |
| | | (1621240 . . . 1629473) |
| EGLN1 | egl nine homolog 1 | RAZU01000110.1 |
| | | (4208348 . . . 4247132) |
| EGLN3 | egl nine homolog 3 | RAZU01000177.1 |
| | | (5266543 . . . 5294174) |
| TP73 | tumor protein p73 | RAZU01000081.1 |
| | | (1570203 . . . 1634595) |
| BAP1 | ubiquitin carboxyl- | RAZU01001824.1 |
| | terminal hydrolase BAP1 | (21442648 . . . 21451297) |
| APC | adenomatous polyposis | RAZU01000093.1 |
| | coli protein | (13195916 . . . 13300691) |
| GSK3B | glycogen synthase | RAZU01000162.1 |
| | kinase-3 beta | (43656984 . . . 43786995) |
| PRKAG2 | 5′-AMP-activated | RAZU01000251.1 |
| | protein kinase subunit | (1132599 . . . 1408050) |
| | gamma-2 | |
| BNIP3 | BCL2/adenovirus E1B | RAZU01000139.1 |
| | 19 kDa protein- | (11390651 . . . 11407722) |
| | interacting protein 3 | |
| PML | protein PML | RAZU01000166.1 |
| | | (5910245 . . . 5943527) |
| AKT1 | RAC-alpha | RAZU01000190.1 |
| | serine/threonine-protein | (2202285 . . . 2223444) |
| | kinase | |
| RIPK3 | receptor-interacting | RAZU01001829.1 |
| | serine/threonine-protein | (22811429 . . . 22815493) |
| | kinase 3 | |
| RIPK1 | receptor-interacting | RAZU01000140.1 |
| | serine/threonine-protein | (1171869 . . . 1203979) |
| | kinase 1 | |
| PPP2CB | Serine/threonine-protein | RAZU01000044.1 |
| | phosphatase 2A catalytic | (18735335 . . . 18754967) |
| | subunit beta | |
| NR3C1 | glucocorticoid receptor | RAZU01000077.1 |
| | | (3604591 . . . 3700275) |
| WEE1 | weel-like protein kinase | RAZU01000142.1 |
| | isoform | (1665360 . . . 1680389) |
| HIPK2 | homeodomain- | RAZU01000045.1 |
| | interacting protein | (12630354 . . . 12820847) |
| | kinase 2 | |
| TRPV4 | transient receptor | RAZU01000171.1 |
| | potential cation channel | (6878000 . . . 6918125) |
| | subfamily V member 4 | |

The term “sirtuin-1” denotes an enzyme that is part of signal transduction in mammals, i.e. the NAD-dependent deacetylase sirtuin-1. Sirtuin-1 is encoded by the SIRT-1 gene. Human sirtuin-1 has the UniProtKB entry Q96EB6. Chinese hamster sirtuin-1 has the UniProtKB entry A0A3L7IF96. The effect of SIRT-1 knockout has been described in PCT/EP2020/067579, which is expressly incorporated herein by reference.

### II. Antibodies

General information regarding the nucleotide sequences of human immunoglobulins light and heavy chains is given in: Kabat, E. A., et al., Sequences of Proteins of Immunological Interest, 5th ed., Public Health Service, National Institutes of Health, Bethesda, Md. (1991).

As used herein, the amino acid positions of all constant regions and domains of the heavy and light chain are numbered according to the Kabat numbering system described in Kabat, et al., Sequences of Proteins of Immunological Interest, 5th ed., Public Health Service, National Institutes of Health, Bethesda, Md. (1991) and is referred to as “numbering according to Kabat” herein. Specifically, the Kabat numbering system (see pages 647-660) of Kabat, et al., Sequences of Proteins of Immunological Interest, 5th ed., Public Health Service, National Institutes of Health, Bethesda, Md. (1991) is used for the light chain constant domain CL of kappa and lambda isotype, and the Kabat EU index numbering system (see pages 661-723) of Kabat, et al., Sequences of Proteins of Immunological Interest, 5th ed., Public Health Service, National Institutes of Health, Bethesda, Md. (1991) is used for the constant heavy chain domains (CH1, hinge, CH2 and CH3, which is herein further clarified by referring to “numbering according to Kabat EU index” in this case).

The term “antibody” herein is used in the broadest sense and encompasses various antibody structures, including but not limited to full length antibodies, monoclonal antibodies, multispecific antibodies (e.g., bispecific antibodies), and antibody-antibody fragment-fusions as well as combinations thereof.

The term “native antibody” denotes naturally occurring immunoglobulin molecules with varying structures. For example, native IgG antibodies are heterotetrameric glycoproteins of about 150,000 Daltons, composed of two identical light chains and two identical heavy chains that are disulfide-bonded. From N- to C-terminus, each heavy chain has a heavy chain variable region (VH) followed by three heavy chain constant domains (CH1, CH2, and CH3), whereby between the first and the second heavy chain constant domain a hinge region is located. Similarly, from N- to C-terminus, each light chain has a light chain variable region (VL) followed by a light chain constant domain (CL). The light chain of an antibody may be assigned to one of two types, called kappa (κ) and lambda (λ), based on the amino acid sequence of its constant domain.

The term “full length antibody” denotes an antibody having a structure substantially similar to that of a native antibody. A full length antibody comprises two full length antibody light chains each comprising in N- to C-terminal direction a light chain variable region and a light chain constant domain, as well as two full length antibody heavy chains each comprising in N- to C-terminal direction a heavy chain variable region, a first heavy chain constant domain, a hinge region, a second heavy chain constant domain and a third heavy chain constant domain. In contrast to a native antibody, a full length antibody may comprise further immunoglobulin domains, such as e.g. one or more additional scFvs, or heavy or light chain Fab fragments, or scFabs conjugated to one or more of the termini of the different chains of the full length antibody, but only a single fragment to each terminus. These conjugates are also encompassed by the term full-length antibody.

The term “antibody binding site” denotes a pair of a heavy chain variable domain and a light chain variable domain. To ensure proper binding to the antigen these variable domains are cognate variable domains, i.e. belong together. An antibody the binding site comprises at least three HVRs (e.g. in case of a VHH) or three-six HVRs (e.g. in case of a naturally occurring, i.e. conventional, antibody with a VH/VL pair). Generally, the amino acid residues of an antibody that are responsible for antigen binding are forming the binding site. These residues are normally contained in a pair of an antibody heavy chain variable domain and a corresponding antibody light chain variable domain. The antigen-binding site of an antibody comprises amino acid residues from the “hypervariable regions” or “HVRs”. “Framework” or “FR” regions are those variable domain regions other than the hypervariable region residues as herein defined. Therefore, the light and heavy chain variable domains of an antibody comprise from N- to C-terminus the regions FR1, HVR1, FR2, HVR2, FR3, HVR3 and FR4. Especially, the HVR3 region of the heavy chain variable domain is the region, which contributes most to antigen binding and defines the binding specificity of an antibody. A “functional binding site” is capable of specifically binding to its target. The term “specifically binding to” denotes the binding of a binding site to its target in an in vitro assay, in certain embodiments, in a binding assay. Such binding assay can be any assay as long the binding event can be detected. For example, an assay in which the antibody is bound to a surface and binding of the antigen(s) to the antibody is measured by Surface Plasmon Resonance (SPR). Alternatively, a bridging ELISA can be used.

The term “hypervariable region” or “HVR”, as used herein, refers to each of the regions of an antibody variable domain comprising the amino acid residue stretches which are hypervariable in sequence (“complementarity determining regions” or “CDRs”) and/or form structurally defined loops (“hypervariable loops”), and/or contain the antigen-contacting residues (“antigen contacts”). Generally, antibodies comprise six HVRs; three in the heavy chain variable domain VH (H1, H2, H3), and three in the light chain variable domain VL (L1, L2, L3).

HVRs Include
(a) hypervariable loops occurring at amino acid residues 26-32 (L1), 50-52 (L2), 91-96 (L3), 26-32 (H1), 53-55 (H2), and 96-101 (H3) (Chothia, C. and Lesk, A. M., J. Mol. Biol. 196 (1987) 901-917);
(b) CDRs occurring at amino acid residues 24-34 (L1), 50-56 (L2), 89-97 (L3), 31-35b (H1), 50-65 (H2), and 95-102 (H3) (Kabat, E. A. et al., Sequences of Proteins of Immunological Interest, 5th ed. Public Health Service, National Institutes of Health, Bethesda, Md. (1991), NIH Publication 91-3242.);
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and
(d) combinations of (a), (b), and/or (c), including amino acid residues 46-56 (L2), 47-56 (L2), 48-56 (L2), 49-56 (L2), 26-35 (H1), 26-35b (H1), 49-65 (H2), 93-102 (H3), and 94-102 (H3).

Unless otherwise indicated, HVR residues and other residues in the variable domain (e.g., FR residues) are numbered herein according to Kabat et al., supra.

The “class” of an antibody refers to the type of constant domains or constant region, preferably the Fc-region, possessed by its heavy chains. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively.

The term “heavy chain constant region” denotes the region of an immunoglobulin heavy chain that contains the constant domains, i.e. the CH1 domain, the hinge region, the CH2 domain and the CH3 domain. In certain embodiments, a human IgG constant region extends from Ala118 to the carboxyl-terminus of the heavy chain (numbering according to Kabat EU index). However, the C-terminal lysine (Lys447) of the constant region may or may not be present (numbering according to Kabat EU index). The term “constant region” denotes a dimer comprising two heavy chain constant regions, which can be covalently linked to each other via the hinge region cysteine residues forming inter-chain disulfide bonds.

The term “heavy chain Fc-region” denotes the C-terminal region of an immunoglobulin heavy chain that contains at least a part of the hinge region (middle and lower hinge region), the CH2 domain and the CH3 domain. In certain embodiments, a human IgG heavy chain Fc-region extends from Asp221, or from Cys226, or from Pro230, to the carboxyl-terminus of the heavy chain (numbering according to Kabat EU index). Thus, an Fc-region is smaller than a constant region but in the C-terminal part identical thereto. However, the C-terminal lysine (Lys447) of the heavy chain Fc-region may or may not be present (numbering according to Kabat EU index). The term “Fc-region” denotes a dimer comprising two heavy chain Fc-regions, which can be covalently linked to each other via the hinge region cysteine residues forming inter-chain disulfide bonds.

The constant region, more precisely the Fc-region, of an antibody (and the constant region likewise) is directly involved in complement activation, C1q binding, C3 activation and Fc receptor binding. While the influence of an antibody on the complement system is dependent on certain conditions, binding to C1q is caused by defined binding sites in the Fc-region. Such binding sites are known in the state of the art and described e.g. by Lukas, T. J., et al., J. Immunol. 127 (1981) 2555-2560; Brunhouse, R., and Cebra, J. J., Mol. Immunol. 16 (1979) 907-917; Burton, D. R., et al., Nature 288 (1980) 338-344; Thommesen, J. E., et al., Mol. Immunol. 37 (2000) 995-1004; Idusogie, E. E., et al., J. Immunol. 164 (2000) 4178-4184; Hezareh, M., et al., J. Virol. 75 (2001) 12161-12168; Morgan, A., et al., Immunology 86 (1995) 319-324; and EP 0 307 434. Such binding sites are e.g. L234, L235, D270, N297, E318, K320, K322, P331 and P329 (numbering according to EU index of Kabat). Antibodies of subclass IgG1, IgG2 and IgG3 usually show complement activation, C1q binding and C3 activation, whereas IgG4 do not activate the complement system, do not bind C1q and do not activate C3. An “Fc-region of an antibody” is a term well known to the skilled artisan and defined on the basis of papain cleavage of antibodies.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, monoclonal antibodies may be made by a variety of techniques, including but not limited to the hybridoma method, recombinant DNA methods, phage-display methods, and methods utilizing transgenic animals containing all or part of the human immunoglobulin loci.

The term “valent” as used within the current application denotes the presence of a specified number of binding sites in an antibody. As such, the terms “bivalent”, “tetravalent”, and “hexavalent” denote the presence of two binding site, four binding sites, and six binding sites, respectively, in an antibody.

A “monospecific antibody” denotes an antibody that has a single binding specificity, i.e. specifically binds to one antigen. Monospecific antibodies can be prepared as full-length antibodies or antibody fragments (e.g. F(ab′)₂) or combinations thereof (e.g. full length antibody plus additional scFv or Fab fragments). A monospecific antibody does not need to be monovalent, i.e. a monospecific antibody may comprise more than one binding site specifically binding to the one antigen. A native antibody, for example, is monospecific but bivalent.

A “multispecific antibody” denotes an antibody that has binding specificities for at least two different epitopes on the same antigen or two different antigens. Multispecific antibodies can be prepared as full-length antibodies or antibody fragments (e.g. F(ab′)₂ bispecific antibodies) or combinations thereof (e.g. full length antibody plus additional scFv or Fab fragments). A multispecific antibody is at least bivalent, i.e. comprises two antigen binding sites. In addition, a multispecific antibody is at least bispecific. Thus, a bivalent, bispecific antibody is the simplest form of a multispecific antibody. Engineered antibodies with two, three or more (e.g. four) functional antigen binding sites have also been reported (see, e.g., US 2002/0004587).

In certain embodiments, the antibody is a multispecific antibody, e.g. at least a bispecific antibody. Multispecific antibodies are monoclonal antibodies that have binding specificities for at least two different antigens or epitopes. In certain embodiments, one of the binding specificities is for a first antigen and the other is for a different second antigen. In certain embodiments, multispecific antibodies may bind to two different epitopes of the same antigen. Multispecific antibodies may also be used to localize cytotoxic agents to cells, which express the antigen.

Multispecific antibodies can be prepared as full-length antibodies or antibody-antibody fragment-fusions.

Techniques for making multispecific antibodies include, but are not limited to, recombinant co-expression of two immunoglobulin heavy chain-light chain pairs having different specificities (see Milstein, C. and Cuello, A. C., Nature 305 (1983) 537-540, WO 93/08829, and Traunecker, A., et al., EMBO J. 10 (1991) 3655-3659), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004); cross-linking two or more antibodies or fragments (see, e.g., U.S. Pat. No. 4,676,980, and Brennan, M., et al., Science 229 (1985) 81-83); using leucine zippers to produce bi-specific antibodies (see, e.g., Kostelny, S. A., et al., J. Immunol. 148 (1992) 1547-1553); using the common light chain technology for circumventing the light chain mis-pairing problem (see, e.g., WO 98/50431); using specific technology for making bispecific antibody fragments (see, e.g., Holliger, P., et al., Proc. Natl. Acad. Sci. USA 90 (1993) 6444-6448); and preparing trispecific antibodies as described, e.g., in Tutt, A., et al., J. Immunol. 147 (1991) 60-69).

Engineered antibodies with three or more antigen binding sites, including for example, “Octopus antibodies”, or DVD-Ig are also included herein (see, e.g., WO 2001/77342 and WO 2008/024715). Other examples of multispecific antibodies with three or more antigen binding sites can be found in WO 2010/115589, WO 2010/112193, WO 2010/136172, WO 2010/145792, and WO 2013/026831. The bispecific antibody or antigen binding fragment thereof also includes a “Dual Acting Fab” or “DAF” (see, e.g., US 2008/0069820 and WO 2015/095539).

Multi-specific antibodies may also be provided in an asymmetric form with a domain crossover in one or more binding arms of the same antigen specificity, i.e. by exchanging the VH/VL domains (see, e.g., WO 2009/080252 and WO 2015/150447), the CH1/CL domains (see, e.g., WO 2009/080253) or the complete Fab arms (see e.g., WO 2009/080251, WO 2016/016299, also see Schaefer et al., Proc. Natl. Acad. Sci. USA 108 (2011) 1187-1191, and Klein at al., MAbs 8 (2016) 1010-1020). In one aspect, the multispecific antibody comprises a Cross-Fab fragment. The term “Cross-Fab fragment” or “xFab fragment” or “crossover Fab fragment” refers to a Fab fragment, wherein either the variable regions or the constant regions of the heavy and light chain are exchanged. A Cross-Fab fragment comprises a polypeptide chain composed of the light chain variable region (VL) and the heavy chain constant region 1 (CH1), and a polypeptide chain composed of the heavy chain variable region (VH) and the light chain constant region (CL). Asymmetrical Fab arms can also be engineered by introducing charged or non-charged amino acid mutations into domain interfaces to direct correct Fab pairing. See e.g., WO 2016/172485.

The antibody or fragment can also be a multispecific antibody as described in WO 2009/080254, WO 2010/112193, WO 2010/115589, WO 2010/136172, WO 2010/145792, or WO 2010/145793.

The antibody or fragment thereof may also be a multispecific antibody as disclosed in WO 2012/163520.

Various further molecular formats for multispecific antibodies are known in the art and are included herein (see e.g., Spiess et al., Mol. Immunol. 67 (2015) 95-106).

Bispecific antibodies are generally antibody molecules that specifically bind to two different, non-overlapping epitopes on the same antigen or to two epitopes on different antigens.

Complex (Multispecific) Antibodies are
a full-length antibody with domain exchange:
a multispecific IgG antibody comprising a first Fab fragment and a second Fab fragment, wherein in the first Fab fragment
a) only the CH1 and CL domains are replaced by each other (i.e. the light chain of the first Fab fragment comprises a VL and a CH1 domain and the heavy chain of the first Fab fragment comprises a VH and a CL domain);
b) only the VH and VL domains are replaced by each other (i.e. the light chain of the first Fab fragment comprises a VH and a CL domain and the heavy chain of the first Fab fragment comprises a VL and a CH1 domain); or
c) the CH1 and CL domains are replaced by each other and the VH and VL domains are replaced by each other (i.e. the light chain of the first Fab fragment comprises a VH and a CH1 domain and the heavy chain of the first Fab fragment comprises a VL and a CL domain); and
wherein the second Fab fragment comprises a light chain comprising a VL and a CL domain, and a heavy chain comprising a VH and a CH1 domain;
the full-length antibody with domain exchange may comprises a first heavy chain including a CH3 domain and a second heavy chain including a CH3 domain, wherein both CH3 domains are engineered in a complementary manner by respective amino acid substitutions, in order to support heterodimerization of the first heavy chain and the modified second heavy chain, e.g. as disclosed in WO 96/27011, WO 98/050431, EP 1870459, WO 2007/110205, WO 2007/147901, WO 2009/089004, WO 2010/129304, WO 2011/90754, WO 2011/143545, WO 2012/058768, WO 2013/157954, or WO 2013/096291 (incorporated herein by reference);
a full-length antibody with domain exchange and additional heavy chain C-terminal binding site:
a multispecific IgG antibody comprising
a) one full length antibody comprising two pairs each of a full length antibody light chain and a full length antibody heavy chain, wherein the binding sites formed by each of the pairs of the full length heavy chain and the full length light chain specifically bind to a first antigen, and
b) one additional Fab fragment, wherein the additional Fab fragment is fused to the C-terminus of one heavy chain of the full length antibody, wherein the binding site of the additional Fab fragment specifically binds to a second antigen,
wherein the additional Fab fragment specifically binding to the second antigen i) comprises a domain crossover such that a) the light chain variable domain (VL) and the heavy chain variable domain (VH) are replaced by each other, or b) the light chain constant domain (CL) and the heavy chain constant domain (CH1) are replaced by each other, or ii) is a single chain Fab fragment;
the one-armed single chain format (=one-armed single chain antibody): antibody comprising a first binding site that specifically binds to a first epitope or antigen and a second binding site that specifically binds to a second epitope or antigen, whereby the individual chains are as follows
   light chain (variable light chain domain+light chain kappa constant domain)
   combined light/heavy chain (variable light chain domain+light chain constant domain+peptidic linker+variable heavy chain domain+CH1+Hinge+CH2+CH3 with knob mutation)
   heavy chain (variable heavy chain domain+CH1+Hinge+CH2+CH3 with hole mutation);
the two-armed single chain format (=two-armed single chain antibody): antibody comprising a first binding site that specifically binds to a first epitope or antigen and a second binding site that specifically binds to a second epitope or antigen, whereby the individual chains are as follows
   combined light/heavy chain 1 (variable light chain domain+light chain constant domain+peptidic linker+variable heavy chain domain+CH1+Hinge+CH2+CH3 with hole mutation)
   combined light/heavy chain 2 (variable light chain domain+light chain constant domain+peptidic linker+variable heavy chain domain+CH1+Hinge+CH2+CH3 with knob mutation);
the common light chain bispecific format (=common light chain bispecific antibody):
antibody comprising a first binding site that specifically binds to a first epitope or antigen and a second binding site that specifically binds to a second epitope or antigen, whereby the individual chains are as follows
   light chain (variable light chain domain+light chain constant domain)
   heavy chain 1 (variable heavy chain domain+CH1+Hinge+CH2+CH3 with hole mutation)
   heavy chain 2 (variable heavy chain domain+CH1+Hinge+CH2+CH3 with knob mutation);
the T-cell bispecific format:
a full-length antibody with additional heavy chain N-terminal binding site with domain exchange comprising
   a first and a second Fab fragment, wherein each binding site of the first and the second Fab fragment specifically bind to a first antigen,
   a third Fab fragment, wherein the binding site of the third Fab fragment specifically binds to a second antigen, and wherein the third Fab fragment comprises a domain crossover such that the variable light chain domain (VL) and the variable heavy chain domain (VH) are replaced by each other, and
   an Fc-region comprising a first Fc-region polypeptide and a second Fc-region polypeptide,
   wherein the first and the second Fab fragment each comprise a heavy chain fragment and a full-length light chain,
   wherein the C-terminus of the heavy chain fragment of the first Fab fragment is fused to the N-terminus of the first Fc-region polypeptide,
   wherein the C-terminus of the heavy chain fragment of the second Fab fragment is fused to the N-terminus of the variable light chain domain of the third Fab fragment and the C-terminus of the CH1 domain of the third Fab fragment is fused to the N-terminus of the second Fc-region polypeptide;
an antibody-multimer-fusions comprising
   (a) an antibody heavy chain and an antibody light chain, and
   (b) a first fusion polypeptide comprising in N- to C-terminal direction a first part of a non-antibody multimeric polypeptide, an antibody heavy chain CH1 domain or an antibody light chain constant domain, an antibody hinge region, an antibody heavy chain CH2 domain and an antibody heavy chain CH3 domain, and a second fusion polypeptide comprising in N- to C-terminal direction the second part of the non-antibody multimeric polypeptide and an antibody light chain constant domain if the first polypeptide comprises an antibody heavy chain CH1 domain or an antibody heavy chain CH1 domain if the first polypeptide comprises an antibody light chain constant domain,
   wherein
      (i) the antibody heavy chain of (a) and the first fusion polypeptide of (b), (ii) the antibody heavy chain of (a) and the antibody light chain of (a), and (iii) the first fusion polypeptide of (b) and the second fusion polypeptide of (b) are each independently of each other covalently linked to each other by at least one disulfide bond,
   wherein
      the variable domains of the antibody heavy chain and the antibody light chain form a binding site specifically binding to an antigen.

The “knobs into holes” dimerization modules and their use in antibody engineering are described in Carter P.; Ridgway J. B. B.; Presta L. G.: Immunotechnology, Volume 2, Number 1, February 1996, pp. 73-73(1).

The CH3 domains in the heavy chains of an antibody can be altered by the “knob-into-holes” technology, which is described in detail with several examples in e.g. WO 96/027011, Ridgway, J. B., et al., Protein Eng. 9 (1996) 617-621; and Merchant, A. M., et al., Nat. Biotechnol. 16 (1998) 677-681. In this method, the interaction surfaces of the two CH3 domains are altered to increase the heterodimerization of these two CH3 domains and thereby of the polypeptide comprising them. Each of the two CH3 domains (of the two heavy chains) can be the “knob”, while the other is the “hole”. The introduction of a disulfide bridge further stabilizes the heterodimers (Merchant, A. M., et al., Nature Biotech. 16 (1998) 677-681; Atwell, S., et al., J. Mol. Biol. 270 (1997) 26-35) and increases the yield.

The mutation T366W in the CH3 domain (of an antibody heavy chain) is denoted as “knob-mutation” or “mutation knob” and the mutations T366S, L368A, Y407V in the CH3 domain (of an antibody heavy chain) are denoted as “hole-mutations” or “mutations hole” (numbering according to Kabat EU index). An additional inter-chain disulfide bridge between the CH3 domains can also be used (Merchant, A. M., et al., Nature Biotech. 16 (1998) 677-681) e.g. by introducing a S354C mutation into the CH3 domain of the heavy chain with the “knob-mutation” (denotes as “knob-cys-mutations” or “mutations knob-cys”) and by introducing a Y349C mutation into the CH3 domain of the heavy chain with the “hole-mutations” (denotes as “hole-cys-mutations” or “mutations hole-cys”) (numbering according to Kabat EU index).

The term “domain crossover” as used herein denotes that in a pair of an antibody heavy chain VH-CH1 fragment and its corresponding cognate antibody light chain, i.e. in an antibody Fab (fragment antigen binding), the domain sequence deviates from the sequence in a native antibody in that at least one heavy chain domain is substituted by its corresponding light chain domain and vice versa. There are three general types of domain crossovers, (i) the crossover of the CH1 and the CL domains, which leads by the domain crossover in the light chain to a VL-CH1 domain sequence and by the domain crossover in the heavy chain fragment to a VH-CL domain sequence (or a full length antibody heavy chain with a VH-CL-hinge-CH2-CH3 domain sequence), (ii) the domain crossover of the VH and the VL domains, which leads by the domain crossover in the light chain to a VH-CL domain sequence and by the domain crossover in the heavy chain fragment to a VL-CH1 domain sequence, and (iii) the domain crossover of the complete light chain (VL-CL) and the complete VH-CH1 heavy chain fragment (“Fab crossover”), which leads to by domain crossover to a light chain with a VH-CH1 domain sequence and by domain crossover to a heavy chain fragment with a VL-CL domain sequence (all aforementioned domain sequences are indicated in N-terminal to C-terminal direction).

As used herein the term “replaced by each other” with respect to corresponding heavy and light chain domains refers to the aforementioned domain crossovers. As such, when CH1 and CL domains are “replaced by each other” it is referred to the domain crossover mentioned under item (i) and the resulting heavy and light chain domain sequence. Accordingly, when VH and VL are “replaced by each other” it is referred to the domain crossover mentioned under item (ii); and when the CH1 and CL domains are “replaced by each other” and the VH and VL domains are “replaced by each other” it is referred to the domain crossover mentioned under item (iii). Bispecific antibodies including domain crossovers are reported, e.g. in WO 2009/080251, WO 2009/080252, WO 2009/080253, WO 2009/080254 and Schaefer, W., et al, Proc. Natl. Acad. Sci. USA 108 (2011) 11187-11192. Such antibodies are generally termed CrossMab.

Multispecific antibodies also comprise in one embodiment at least one Fab fragment including either a domain crossover of the CH1 and the CL domains as mentioned under item (i) above, or a domain crossover of the VH and the VL domains as mentioned under item (ii) above, or a domain crossover of the VH-CH1 and the VL-VL domains as mentioned under item (iii) above. In case of multispecific antibodies with domain crossover, the Fabs specifically binding to the same antigen(s) are constructed to be of the same domain sequence. Hence, in case more than one Fab with a domain crossover is contained in the multispecific antibody, said Fab(s) specifically bind to the same antigen.

A “humanized” antibody refers to an antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., the CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “recombinant antibody”, as used herein, denotes all antibodies (chimeric, humanized and human) that are prepared, expressed, created or isolated by recombinant means, such as recombinant cells. This includes antibodies isolated from recombinant cells such as NS0, HEK, BHK, amniocyte or CHO cells.

As used herein, the term “antibody fragment” refers to a molecule other than an intact antibody that comprises a portion of an intact antibody that binds the antigen to which the intact antibody binds, i.e. it is a functional fragment. Examples of antibody fragments include but are not limited to Fv; Fab; Fab′; Fab′-SH; F(ab′)2; bispecific Fab; diabodies; linear antibodies; single-chain antibody molecules (e.g., scFv or scFab).

### III. Recombinant Methods and Compositions

Antibodies may be produced using recombinant methods and compositions, e.g., as described in U.S. Pat. No. 4,816,567. For these methods, one or more isolated nucleic acid(s) encoding an antibody are provided.

In one aspect, a method of making an antibody is provided, wherein the method comprises culturing a host cell comprising nucleic acid(s) encoding the antibody, as provided above, under conditions suitable for expression of the antibody, and optionally recovering the antibody from the host cell (or host cell culture medium).

For recombinant production of an antibody, nucleic acids encoding the antibody, e.g., as described above, are isolated and inserted into one or more vectors for further cloning and/or expression in a host cell. Such nucleic acids may be readily isolated and sequenced using conventional procedures (e.g., by using oligonucleotide probes that are capable of binding specifically to genes encoding the heavy and light chains of the antibody) or produced by recombinant methods or obtained by chemical synthesis.

Generally, for the recombinant large-scale production of a polypeptide of interest, such as e.g. a therapeutic antibody, a cell stably expressing and secreting said polypeptide is required. This cell is termed “recombinant cell” or “recombinant production cell” and the process used for generating such a cell is termed “cell line development”. In the first step of the cell line development process, a suitable host cell, such as e.g. a CHO cell, is transfected with a nucleic acid sequence suitable for expression of said polypeptide of interest. In a second step, a cell stably expressing the polypeptide of interest is selected based on the co-expression of a selection marker, which had been co-transfected with the nucleic acid encoding the polypeptide of interest.

A nucleic acid encoding a polypeptide, i.e. the coding sequence, is denoted as a structural gene. Such a structural gene is pure coding information. Thus, additional regulatory elements are required for expression thereof. Therefore, normally a structural gene is integrated in a so-called expression cassette. The minimal regulatory elements needed for an expression cassette to be functional in a mammalian cell are a promoter functional in said mammalian cell, which is located upstream, i.e. 5′, to the structural gene, and a polyadenylation signal sequence functional in said mammalian cell, which is located downstream, i.e. 3′, to the structural gene. The promoter, the structural gene and the polyadenylation signal sequence are arranged in an operably linked form.

In case the polypeptide of interest is a heteromultimeric polypeptide that is composed of different (monomeric) polypeptides, such as e.g. an antibody or a complex antibody format, not only a single expression cassette is required but a multitude of expression cassettes differing in the contained structural gene, i.e. at least one expression cassette for each of the different (monomeric) polypeptides of the heteromultimeric polypeptide. For example, a full-length antibody is a heteromultimeric polypeptide comprising two copies of a light chain as well as two copies of a heavy chain. Thus, a full-length antibody is composed of two different polypeptides. Therefore, two expression cassettes are required for the expression of a full-length antibody, one for the light chain and one for the heavy chain. If, for example, the full-length antibody is a bispecific antibody, i.e. the antibody comprises two different binding sites specifically binding to two different antigens, the two light chains as well as the two heavy chains are also different from each other. Thus, such a bispecific, full-length antibody is composed of four different polypeptides and therefore, four expression cassettes are required.

The expression cassette(s) for the polypeptide of interest is(are) in turn integrated into one or more so called “expression vector(s)”. An “expression vector” is a nucleic acid providing all required elements for the amplification of said vector in bacterial cells as well as the expression of the comprised structural gene(s) in a mammalian cell. Typically, an expression vector comprises a prokaryotic plasmid propagation unit, e.g. for E. coli, comprising an origin of replication, and a prokaryotic selection marker, as well as a eukaryotic selection marker, and the expression cassettes required for the expression of the structural gene(s) of interest. An “expression vector” is a transport vehicle for the introduction of expression cassettes into a mammalian cell.

As outlined in the previous paragraphs, the more complex the polypeptide to be expressed is the higher also the number of required different expression cassettes is. Inherently with the number of expression cassettes also the size of the nucleic acid to be integrated into the genome of the host cell increases. Concomitantly also the size of the expression vector increases. However, there is a practical upper limit to the size of a vector in the range of about 15 kbps above which handling and processing efficiency profoundly drops. This issue can be addressed by using two or more expression vectors. Thereby the expression cassettes can be split between different expression vectors each comprising only some of the expression cassettes resulting in a size reduction.

Cell line development (CLD) for the generation of recombinant cell expressing a heterologous polypeptide, such as e.g. a multispecific antibody, employs either random integration (RI) or targeted integration (TI) of the nucleic acid(s) comprising the respective expression cassettes required for the expression and production of the heterologous polypeptide of interest.

Using RI, in general, several vectors or fragments thereof integrate into the cell's genome at the same or different loci.

Using TI, in general, a single copy of the transgene comprising the different expression cassettes is integrated at a predetermined “hot-spot” in the host cell's genome.

Suitable host cells for the expression of an (glycosylated) antibody are generally derived from multicellular organisms such as e.g. vertebrates.

### IV. Host Cells

Any mammalian cell line that is adapted to grow in suspension can be used in the method according to the current invention. In addition, independent from the integration method, i.e. for RI as well as TI, any mammalian host cell can be used.

Examples of useful mammalian host cell lines are human amniocyte cells (e.g. CAP-T cells as described in Woelfel, J. et al., BMC Proc. 5 (2011) P133); monkey kidney CV1 line transformed by SV40 (COS-7); human embryonic kidney line (HEK293 or HEK293T cells as described, e.g., in Graham, F. L. et al., J. Gen Virol. 36 (1977) 59-74); baby hamster kidney cells (BHK); mouse sertoli cells (TM4 cells as described, e.g., in Mather, J. P., Biol. Reprod. 23 (1980) 243-252); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells (as described, e.g., in Mather, J. P. et al., Annals N.Y. Acad. Sci. 383 (1982) 44-68); MRC 5 cells; and FS4 cells. Other useful mammalian host cell lines include Chinese hamster ovary (CHO) cells, including DHFR-CHO cells (Urlaub, G. et al., Proc. Natl. Acad. Sci. USA 77 (1980) 4216-4220); and myeloma cell lines such as Y0, NS0 and Sp2/0. For a review of certain mammalian host cell lines suitable for antibody production, see, e.g., Yazaki, P. and Wu, A M., Methods in Molecular Biology, Vol. 248, Lo, B. K. C. (ed.), Humana Press, Totowa, N.J. (2004), pp. 255-268.

In certain embodiments, the mammalian host cell is, e.g., a Chinese Hamster Ovary (CHO) cell (e.g. CHO K1, CHO DG44, etc.), a Human Embryonic Kidney (HEK) cell, a lymphoid cell (e.g., Y0, NS0, Sp2/0 cell), or a human amniocyte cells (e.g. CAP-T, etc.). In one preferred embodiment, the mammalian (host) cell is a CHO cell.

Targeted integration allows exogenous nucleotide sequences to be integrated into a pre-determined site of a mammalian cell's genome. In certain embodiments, the targeted integration is mediated by a recombinase that recognizes one or more recombination recognition sequences (RRSs), which are present in the genome and in the exogenous nucleotide sequence to be integrated. In certain embodiments, the targeted integration is mediated by homologous recombination.

A “recombination recognition sequence” (RRS) is a nucleotide sequence recognized by a recombinase and is necessary and sufficient for recombinase-mediated recombination events. A RRS can be used to define the position where a recombination event will occur in a nucleotide sequence.

In certain embodiments, a RRS can be recognized by a Cre recombinase. In certain embodiments, a RRS can be recognized by a FLP recombinase. In certain embodiments, a RRS can be recognized by a Bxb1 integrase. In certain embodiments, a RRS can be recognized by a φC31 integrase.

In certain embodiments when the RRS is a LoxP site, the cell requires the Cre recombinase to perform the recombination. In certain embodiments when the RRS is a FRT site, the cell requires the FLP recombinase to perform the recombination. In certain embodiments when the RRS is a Bxb1 attP or a Bxb1 attB site, the cell requires the Bxb1 integrase to perform the recombination. In certain embodiments when the RRS is a φC31 attP or a φC31 attB site, the cell requires the φC31 integrase to perform the recombination. The recombinases can be introduced into a cell using an expression vector comprising coding sequences of the enzymes or as protein or a mRNA.

With respect to TI, any known or future mammalian host cell suitable for TI comprising a landing site as described herein integrated at a single site within a locus of the genome can be used in the current invention. Such a cell is denoted as mammalian TI host cell. In certain embodiments, the mammalian TI host cell is a hamster cell, a human cell, a rat cell, or a mouse cell comprising a landing site as described herein. In one preferred embodiment, the mammalian TI host cell is a CHO cell. In certain embodiments, the mammalian TI host cell is a Chinese hamster ovary (CHO) cell, a CHO K1 cell, a CHO K1SV cell, a CHO DG44 cell, a CHO DUKXB-11 cell, a CHO K1S cell, or a CHO K1M cell comprising a landing site as described herein integrated at a single site within a locus of the genome.

In certain embodiments, a mammalian TI host cell comprises an integrated landing site, wherein the landing site comprises one or more recombination recognition sequence (RRS). The RRS can be recognized by a recombinase, for example, a Cre recombinase, an FLP recombinase, a Bxb1 integrase, or a φC31 integrase. The RRS can be selected independently of each other from the group consisting of a LoxP sequence, a LoxP L3 sequence, a LoxP 2L sequence, a LoxFas sequence, a Lox511 sequence, a Lox2272 sequence, a Lox2372 sequence, a Lox5171 sequence, a Loxm2 sequence, a Lox71 sequence, a Lox66 sequence, a FRT sequence, a Bxb1 attP sequence, a Bxb1 attB sequence, a φC31 attP sequence, and a φC31 attB sequence. If multiple RRSs have to be present, the selection of each of the sequences is dependent on the other insofar as non-identical RRSs are chosen.

In certain embodiments, the landing site comprises one or more recombination recognition sequence (RRS), wherein the RRS can be recognized by a recombinase. In certain embodiments, the integrated landing site comprises at least two RRSs. In certain embodiments, an integrated landing site comprises three RRSs, wherein the third RRS is located between the first and the second RRS. In certain preferred embodiments, all three RRSs are different. In certain embodiments, the landing site comprises a first, a second and a third RRS, and at least one selection marker located between the first and the second RRS, and the third RRS is different from the first and/or the second RRS. In certain embodiments, the landing site further comprises a second selection marker, and the first and the second selection markers are different. In certain embodiments, the landing site further comprises a third selection marker and an internal ribosome entry site (IRES), wherein the IRES is operably linked to the third selection marker. The third selection marker can be different from the first or the second selection marker.

Although the invention is exemplified with a CHO cell hereafter, this is presented solely to exemplify the invention but shall not be construed in any way as limitation. The true scope of the invention is set forth in the claims.

An exemplary mammalian TI host cell that is suitable for use in a method according to the current invention is a CHO cell harboring a landing site integrated at a single site within a locus of its genome wherein the landing site comprises three heterospecific loxP sites for Cre recombinase mediated DNA recombination.

In this example, the heterospecific loxP sites are L3, LoxFas and 2L (see e.g. Lanza et al., Biotechnol. J. 7 (2012) 898-908; Wong et al., Nucleic Acids Res. 33 (2005) e147), whereby L3 and 2L flank the landing site at the 5′-end and 3′-end, respectively, and LoxFas is located between the L3 and 2L sites. The landing site further contains a bicistronic unit linking the expression of a selection marker via an IRES to the expression of the fluorescent GFP protein allowing to stabilize the landing site by positive selection as well as to select for the absence of the site after transfection and Cre-recombination (negative selection). Green fluorescence protein (GFP) serves for monitoring the RMCE reaction.

Such a configuration of the landing site as outlined in the previous paragraph allows for the simultaneous integration of two vectors, e.g. of a so called front vector harboring an L3 and a LoxFas site and a back vector harboring a LoxFas and an 2L site. The functional elements of a selection marker gene different from that present in the landing site can be distributed between both vectors: promoter and start codon can be located on the front vector whereas coding region and poly A signal are located on the back vector. Only correct recombinase-mediated integration of said nucleic acids from both vectors induces resistance against the respective selection agent.

Generally, a mammalian TI host cell is a mammalian cell comprising a landing site integrated at a single site within a locus of the genome of the mammalian cell, wherein the landing site comprises a first and a second recombination recognition sequence flanking at least one first selection marker, and a third recombination recognition sequence located between the first and the second recombination recognition sequence, and all the recombination recognition sequences are different.

The selection marker(s) can be selected from the group consisting of an aminoglycoside phosphotransferase (APH) (e.g., hygromycin phosphotransferase (HYG), neomycin and G418 APH), dihydrofolate reductase (DHFR), thymidine kinase (TK), glutamine synthetase (GS), asparagine synthetase, tryptophan synthetase (indole), histidinol dehydrogenase (histidinol D), and genes encoding resistance to puromycin, blasticidin, bleomycin, phleomycin, chloramphenicol, Zeocin, and mycophenolic acid. The selection marker(s) can also be a fluorescent protein selected from the group consisting of green fluorescent protein (GFP), enhanced GFP (eGFP), a synthetic GFP, yellow fluorescent protein (YFP), enhanced YFP (eYFP), cyan fluorescent protein (CFP), mPlum, mCherry, tdTomato, mStrawberry, J-red, DsRed-monomer, mOrange, mKO, mCitrine, Venus, YPet, Emerald6, CyPet, mCFPm, Cerulean, and T-Sapphire.

An exogenous nucleotide sequence is a nucleotide sequence that does not originate from a specific cell but can be introduced into said cell by DNA delivery methods, such as, e.g., by transfection, electroporation, or transformation methods. In certain embodiments, a mammalian TI host cell comprises at least one landing site integrated at one or more integration sites in the mammalian cell's genome. In certain embodiments, the landing site is integrated at one or more integration sites within a specific a locus of the genome of the mammalian cell.

In certain embodiments, the integrated landing site comprises at least one selection marker. In certain embodiments, the integrated landing site comprises a first, a second and a third RRS, and at least one selection marker. In certain embodiments, a selection marker is located between the first and the second RRS. In certain embodiments, two RRSs flank at least one selection marker, i.e., a first RRS is located 5′ (upstream) and a second RRS is located 3′ (downstream) of the selection marker. In certain embodiments, a first RRS is adjacent to the 5′-end of the selection marker and a second RRS is adjacent to the 3′-end of the selection marker. In certain embodiments, the landing site comprises a first, second, and third RRS, and at least one selection marker located between the first and the third RRS.

In certain embodiments, a selection marker is located between a first and a second RRS and the two flanking RRSs are different. In certain preferred embodiments, the first flanking RRS is a LoxP L3 sequence and the second flanking RRS is a LoxP 2L sequence. In certain embodiments, a LoxP L3 sequenced is located 5′ of the selection marker and a LoxP 2L sequence is located 3′ of the selection marker. In certain embodiments, the first flanking RRS is a wild-type FRT sequence and the second flanking RRS is a mutant FRT sequence. In certain embodiments, the first flanking RRS is a Bxb1 attP sequence and the second flanking RRS is a Bxb1 attB sequence. In certain embodiments, the first flanking RRS is a φC31 attP sequence and the second flanking RRS is a φC31 attB sequence. In certain embodiments, the two RRSs are positioned in the same orientation. In certain embodiments, the two RRSs are both in the forward or reverse orientation. In certain embodiments, the two RRSs are positioned in opposite orientation.

In certain embodiments, the integrated landing site comprises a first and a second selection marker, which are flanked by two RRSs, wherein the first selection marker is different from the second selection marker. In certain embodiments, the two selection markers are both independently of each other selected from the group consisting of a glutamine synthetase selection marker, a thymidine kinase selection marker, a HYG selection marker, and a puromycin resistance selection marker. In certain embodiments, the integrated landing site comprises a thymidine kinase selection marker and a HYG selection marker. In certain embodiments, the first selection maker is selected from the group consisting of an aminoglycoside phosphotransferase (APH) (e.g., hygromycin phosphotransferase (HYG), neomycin and G418 APH), dihydrofolate reductase (DHFR), thymidine kinase (TK), glutamine synthetase (GS), asparagine synthetase, tryptophan synthetase (indole), histidinol dehydrogenase (histidinol D), and genes encoding resistance to puromycin, blasticidin, bleomycin, phleomycin, chloramphenicol, Zeocin, and mycophenolic acid, and the second selection maker is selected from the group consisting of a GFP, an eGFP, a synthetic GFP, a YFP, an eYFP, a CFP, an mPlum, an mCherry, a tdTomato, an mStrawberry, a J-red, a DsRed-monomer, an mOrange, an mKO, an mCitrine, a Venus, a YPet, an Emerald, a CyPet, an mCFPm, a Cerulean, and a T-Sapphire fluorescent protein. In certain embodiments, the first selection marker is a glutamine synthetase selection marker and the second selection marker is a GFP fluorescent protein. In certain embodiments, the two RRSs flanking both selection markers are different.

In certain embodiments, the selection marker is operably linked to a promoter sequence. In certain embodiments, the selection marker is operably linked to an SV40 promoter. In certain embodiments, the selection marker is operably linked to a human Cytomegalovirus (CMV) promoter.

### V. Targeted Integration

One method for the generation of a recombinant mammalian cell according to the current invention is targeted integration (TI).

In targeted integration, site-specific recombination is employed for the introduction of an exogenous nucleic acid into a specific locus in the genome of a mammalian TI host cell. This is an enzymatic process wherein a sequence at the site of integration in the genome is exchanged for the exogenous nucleic acid. One system used to effect such nucleic acid exchanges is the Cre-lox system. The enzyme catalyzing the exchange is the Cre recombinase. The sequence to be exchanged is defined by the position of two lox(P)-sites in the genome as well as in the exogenous nucleic acid. These lox(P)-sites are recognized by the Cre recombinase. Nothing more is required, i.e. no ATP etc. Originally, the Cre-lox system has been found in bacteriophage P1.

The Cre-lox system operates in different cell types, like mammals, plants, bacteria and yeast.

In certain embodiments, the exogenous nucleic acid encoding the heterologous polypeptide has been integrated into the mammalian TI host cell by single or double recombinase mediated cassette exchange (RMCE). Thereby a recombinant mammalian cell, such as a recombinant CHO cell, is obtained, in which a defined and specific expression cassette sequence has been integrated into the genome at a single locus, which in turn results in the efficient expression and production of the heterologous polypeptide.

The Cre-LoxP site-specific recombination system has been widely used in many biological experimental systems. Cre recombinase is a 38-kDa site-specific DNA recombinase that recognizes 34 bp LoxP sequences. Cre recombinase is derived from bacteriophage P1 and belongs to the tyrosine family site-specific recombinase. Cre recombinase can mediate both intra and intermolecular recombination between LoxP sequences. The LoxP sequence is composed of an 8 bp non-palindromic core region flanked by two 13 bp inverted repeats. Cre recombinase binds to the 13 bp repeat thereby mediating recombination within the 8 bp core region. Cre-LoxP-mediated recombination occurs at a high efficiency and does not require any other host factors. If two LoxP sequences are placed in the same orientation on the same nucleotide sequence, Cre recombinase-mediated recombination will excise DNA sequences located between the two LoxP sequences as a covalently closed circle. If two LoxP sequences are placed in an inverted position on the same nucleotide sequence, Cre recombinase-mediated recombination will invert the orientation of the DNA sequences located between the two sequences. If two LoxP sequences are on two different DNA molecules and if one DNA molecule is circular, Cre recombinase-mediated recombination will result in integration of the circular DNA sequence.

The term “matching RRSs” indicates that a recombination occurs between two RRSs. In certain embodiments, the two matching RRSs are the same. In certain embodiments, both RRSs are wild-type LoxP sequences. In certain embodiments, both RRSs are mutant LoxP sequences. In certain embodiments, both RRSs are wild-type FRT sequences. In certain embodiments, both RRSs are mutant FRT sequences. In certain embodiments, the two matching RRSs are different sequences but can be recognized by the same recombinase. In certain embodiments, the first matching RRS is a Bxb1 attP sequence and the second matching RRS is a Bxb1 attB sequence.

In certain embodiments, the first matching RRS is a φC31 attB sequence and the second matching RRS is a φC31 attB sequence.

A “two-plasmid RMCE” strategy or “double RMCE” is employed in the method according to the current invention when using a two-vector combination. For example, but not by way of limitation, an integrated landing site could comprise three RRSs, e.g., an arrangement where the third RRS (“RRS3”) is present between the first RRS (“RRS1”) and the second RRS (“RRS2”), while a first vector comprises two RRSs matching the first and the third RRS on the integrated exogenous nucleotide sequence, and a second vector comprises two RRSs matching the third and the second RRS on the integrated exogenous nucleotide sequence.

The two-plasmid RMCE strategy involves using three RRS sites to carry out two independent RMCEs simultaneously. Therefore, a landing site in the mammalian TI host cell using the two-plasmid RMCE strategy includes a third RRS site (RRS3) that has no cross activity with either the first RRS site (RRS1) or the second RRS site (RRS2). The two plasmids to be targeted require the same flanking RRS sites for efficient targeting, one plasmid (front) flanked by RRS1 and RRS3 and the other (back) by RRS3 and RRS2. In addition, two selection markers are needed in the two-plasmid RMCE. One selection marker expression cassette was split into two parts. The front plasmid would contain the promoter followed by a start codon and the RRS3 sequence. The back plasmid would have the RRS3 sequence fused to the N-terminus of the selection marker coding region, minus the start-codon (ATG). Additional nucleotides may need to be inserted between the RRS3 site and the selection marker sequence to ensure in frame translation for the fusion protein, i.e. operable linkage. Only when both plasmids are correctly inserted, the full expression cassette of the selection marker will be assembled and, thus, rendering cells resistance to the respective selection agent.

Two-plasmid RMCE involves double recombination cross-over events, catalyzed by a recombinase, between the two heterospecific RRSs within the target genomic locus and the donor DNA molecule. Two-plasmid RMCE is designed to introduce a copy of the DNA sequences from the front- and back-vector in combination into the pre-determined locus of a mammalian TI host cell's genome. RMCE can be implemented such that prokaryotic vector sequences are not introduced into the mammalian TI host cell's genome, thus, reducing and/or preventing unwanted triggering of host immune or defense mechanisms. The RMCE procedure can be repeated with multiple DNA sequences.

In certain embodiments, targeted integration is achieved by two RMCEs, wherein two different DNA sequences, each comprising at least one expression cassette encoding a part of a heteromultimeric polypeptide and/or at least one selection marker or part thereof flanked by two heterospecific RRSs, are both integrated into a pre-determined site of the genome of a RRSs matching mammalian TI host cell. In certain embodiments, targeted integration is achieved by multiple RMCEs, wherein DNA sequences from multiple vectors, each comprising at least one expression cassette encoding a part of a heteromultimeric polypeptide and/or at least one selection marker or part thereof flanked by two heterospecific RRSs, are all integrated into a predetermined site of the genome of a mammalian TI host cell. In certain embodiments the selection marker can be partially encoded on the first the vector and partially encoded on the second vector such that only the correct integration of both by double RMCE allows for the expression of the selection marker.

In certain embodiments, targeted integration via recombinase-mediated recombination leads to selection marker and/or the different expression cassettes for the multimeric polypeptide integrated into one or more pre-determined integration sites of a host cell genome free of sequences from a prokaryotic vector.

It has to be pointed out that, as in certain embodiments, knockout can be performed either before introduction of the exogenous nucleic acid encoding the heterologous polypeptide or thereafter.

### VI. Compositions and Methods According to the Invention

Herein is reported a method for generating a recombinant mammalian cell expressing a heterologous polypeptide and a method for producing a heterologous polypeptide using said recombinant mammalian cell, wherein in the recombinant mammalian cell the activity/function/expression of at least one of the genes from the group consisting of MYC, STK11, SMAD4, PPP2CB, RBM38, NF1, CDK12, SIN3A, PARP-1, ATM, Hipk2, BARD1, HIF1AN, SMAD3, PALB2, FUBP1, RBL2, RPS6KA3, GPS2, ETS1, E2F5, CDKN1A, RNF43, EEF2K, AKT1, BRCA1, ATR, SMAD2, BAD, FOXO1, PBRM1, NRAS, BRCA2, NOTCH1, CREBBP, and RBX1 has been reduced/eliminated/diminished/(completely) knocked-out.

The invention is based, at least in part, on the finding that the knockout of at least one of the genes from the group consisting of MYC, STK11, SMAD4, PPP2CB, RBM38, NF1, CDK12, SIN3A, PARP-1, ATM, Hipk2, BARD1, HIF1AN, SMAD3, PALB2, FUBP1, RBL2, RPS6KA3, GPS2, ETS1, E2F5, CDKN1A, RNF43, EEF2K, AKT1, BRCA1, ATR, SMAD2, BAD, FOXO1, PBRM1, NRAS, BRCA2, NOTCH1, CREBBP, and RBX1, in a mammalian cell, e.g. such as CHO cells, improves recombinant productivity, e.g. of standard IgG-type antibodies and especially of complex antibody formats.

The invention is exemplified in the following using an exemplary cell line and exemplary heterologous polypeptides. However, any cell suitable for heterologous polypeptide expression can be used. The invention is further exemplified using CRISPR-Cas9-mediated gene knockout. However, any method or technique for reducing or disrupting the target gene may be used, e.g. RNAi, zinc finger or TALEN proteins. Thus, all of this is presented as mere exemplification of the general concept underlying the current invention and shall not be construed as limitation thereof. The true scope of the invention is set forth in the appended claims.

As exemplary cell line a CHO cell line previously generated and having suitable performance for large scale therapeutic protein production was used (see, e.g., WO 2019/126634).

Different individual gene knockouts (KOs) were introduced into two antibody-producing CHO cell lines. One cell line produced an anti-PD1-antibody-IL2v fusion and the other produced an anti-FAP antibody-CD137 fusion.

The knockouts were generated using CRISPR-Cas9. For CRISPR-Cas9-mediated gene knockout, three different sites within the coding sequence (CDS) of the respective gene using three different gRNAs were targeted at the same time using multiplexed ribonucleoprotein delivery. Multiplexed ribonucleoprotein delivery shows higher gene-editing efficacy and specificity compared to the common plasmid based CRISPR-Cas9 editing. Double-strand breaks at the gene target sites induce indel formations or due to multiplexed gRNA usage also deletions of exons result in a frameshift of the CDS of the target protein.

The following genes were individually knocked-out:

MYC, STK11, SMAD4, PPP2CB, RBM38, NF1, CDK12, SIN3A, PARP-1, ATM, HIPK2, BARD1, HIF1AN, SMAD3, LATS2, NF2, PALB2, TP73, FUBP1, NR3C1, PIK3R1, PTCH1, APC, TRPV4, PML, RPS6KA3, RBP2, EGLN2, MAPK14, GPS2, ETS1, E2F5, JUN, p53, CDKN2D, LATS1, NFKBIA, GSK3B, CDKN1A, CDKN2A, RNF43, HTATIP2, EEF2K, RBP1, BNIP3, AKT1, HIF1A, EPHA2, KEAP1, MAPK8IP3, ERK1/MAPK3, ERK2/MAPK1, E2F1, CDKN1C, NUPR1, CAMK1, BAP1, CHK2, CDKN2C, BRCA1, RASA1, RIPK3, EGLN3, ERKS/MAPK7, RPS6KA5, MAPK9, CDKN1B, MXI1, PRKAG2, ATR, SMAD2, FOXO3, BAD, EIF4EBP1, E2F7, FOXO1, CTNNB1, PBRM1, NRAS, BRCA2, NOTCH1, AJUBA, MAPK8, FBXW7, EGLN1, RIPK1, VHL, CREBBP, CHK1, RBX1, CUL3, WEE1.

To show the influence of the respective knockout on antibody productivity and growth, a 4-day batch cultivation was performed (see Example 7). Included were controls for CRISPR-Cas9 on-target efficiency and a non-target control. The cellular densities used in fermentation processes increase steadily. The results are presented in the following Table:

| | | | normalized | Normalized |
|---|---|---|---|---|
| | IgG [mg/L]- | IgG[mg/L]- | IgG- | IgG-PD1- |
| Gene | FAPcd137 | PD1-IL2v | FAPcd137 | IL2v |
| MYC | 588.30 | 711.20 | 199.56 | 149.13 |
| STK11 | 381.60 | 581.50 | 129.44 | 121.93 |
| SMAD4 | 381.60 | 558.50 | 129.44 | 117.11 |
| PPP2CB | 378.70 | 536.80 | 128.46 | 112.56 |
| RBM38 | 337.90 | 558.50 | 114.62 | 117.11 |
| NF1 | 334.80 | 487.70 | 113.57 | 102.26 |
| CDK12 | 332.00 | 595.10 | 112.62 | 124.79 |
| SIN3A | 322.60 | 440.80 | 109.43 | 92.43 |
| PARP-1 | 321.00 | 545.70 | 108.89 | 114.43 |
| ATM | 316.80 | 516.40 | 107.46 | 108.28 |
| Hipk2 | 314.80 | 522.40 | 106.78 | 109.54 |
| BARD1 | 312.90 | 569.00 | 106.14 | 119.31 |
| HIF1AN | 312.10 | 463.10 | 105.87 | 97.11 |
| SMAD3 | 308.60 | 507.20 | 104.68 | 106.35 |
| LATS2 | 304.80 | 463.60 | 103.39 | 97.21 |
| NF2 | 304.40 | 428.70 | 103.26 | 89.89 |
| PALB2 | 302.90 | 502.20 | 102.75 | 105.31 |
| TP73 | 302.50 | 439.50 | 102.61 | 92.16 |
| FUBP1 | 299.80 | 504.90 | 101.70 | 105.87 |
| NR3C1 | 299.80 | 482.70 | 101.70 | 101.22 |
| PIK3R1 | 299.40 | 477.30 | 101.56 | 100.08 |
| PTCH1 | 299.40 | 474.70 | 101.56 | 99.54 |
| APC | 297.90 | 427.40 | 101.05 | 89.62 |
| TRPV4 | 296.40 | 465.80 | 100.54 | 97.67 |
| PML | 296.00 | 489.50 | 100.41 | 102.64 |
| RPS6KA3 | 295.60 | 503.50 | 100.27 | 105.58 |
| RBP2 | 295.20 | 518.20 | 100.14 | 108.66 |
| negative | 294.80 | 476.90 | 100.00 | 100.00 |
| control | | | | |
| EGLN2 | 294.10 | 478.70 | 99.76 | 100.38 |
| MAPK14 | 293.70 | 463.60 | 99.63 | 97.21 |
| GPS2 | 293.70 | 521.90 | 99.63 | 109.44 |
| ETS1 | 292.60 | 544.80 | 99.25 | 114.24 |
| E2F5 | 291.80 | 537.30 | 98.98 | 112.67 |
| JUN | 289.20 | 491.30 | 98.10 | 103.02 |
| p53 | 287.60 | 503.50 | 97.56 | 105.58 |
| CDKN2D | 287.30 | 486.30 | 97.46 | 101.97 |
| LATS1 | 286.10 | 446.50 | 97.05 | 93.63 |
| NFKBIA | 285.80 | 468.00 | 96.95 | 98.13 |
| GSK3B | 285.00 | 426.20 | 96.68 | 89.37 |
| CDKN1A | 283.90 | 504.90 | 96.30 | 105.87 |
| CDKN2A | 283.10 | 469.80 | 96.03 | 98.51 |
| RNF43 | 282.70 | 525.10 | 95.90 | 110.11 |
| HTATIP2 | 282.00 | 35.20 | 95.66 | 7.38 |
| EEF2K | 281.60 | 533.50 | 95.52 | 111.87 |
| RBP1 | 279.40 | 429.60 | 94.78 | 90.08 |
| BNIP3 | 278.20 | 459.60 | 94.37 | 96.37 |
| AKT1 | 278.20 | 533.50 | 94.37 | 111.87 |
| HIF1A | 276.70 | 475.60 | 93.86 | 99.73 |
| EPHA2 | 276.40 | 457.40 | 93.76 | 95.91 |
| KEAP1 | 276.00 | 406.20 | 93.62 | 85.18 |
| MAPK8IP3 | 274.10 | 460.50 | 92.98 | 96.56 |
| ERK1/MAPK3 | 273.80 | 477.80 | 92.88 | 100.19 |
| ERK2/MAPK1 | 273.80 | 450.80 | 92.88 | 94.53 |
| E2F1 | 273.80 | — | 92.88 | — |
| CDKN1C | 272.30 | 483.20 | 92.37 | 101.32 |
| NUPR1 | 271.10 | 495.80 | 91.96 | 103.96 |
| CAMK1 | 270.80 | 485.40 | 91.86 | 101.78 |
| BAP1 | 270.40 | 472.00 | 91.72 | 98.97 |
| CHK2 | 270.00 | 440.80 | 91.59 | 92.43 |
| CDKN2C | 270.00 | 433.90 | 91.59 | 90.98 |
| BRCA1 | 268.90 | 550.40 | 91.21 | 115.41 |
| RASA1 | 268.60 | 440.80 | 91.11 | 92.43 |
| RIPK3 | 268.20 | 452.10 | 90.98 | 94.80 |
| EGLN3 | 267.80 | 472.00 | 90.84 | 98.97 |
| ERK5/MAPK7 | 267.10 | 457.00 | 90.60 | 95.83 |
| RPS6KA5 | 264.50 | 468.00 | 89.72 | 98.13 |
| MAPK9 | 264.50 | 463.10 | 89.72 | 97.11 |
| CDKN1B | 264.10 | 419.70 | 89.59 | 88.01 |
| MXI1 | 261.20 | 461.40 | 88.60 | 96.75 |
| PRKAG2 | 260.80 | 470.70 | 88.47 | 98.70 |
| ATR | 255.70 | 512.70 | 86.74 | 107.51 |
| SMAD2 | 248.40 | 518.20 | 84.26 | 108.66 |
| FOXO3 | 248.00 | 489.90 | 84.12 | 102.73 |
| BAD | 246.90 | 550.90 | 83.75 | 115.52 |
| EIF4EBP1 | 246.90 | 490.80 | 83.75 | 102.91 |
| E2F7 | 246.50 | 488.10 | 83.62 | 102.35 |
| FOXO1 | 246.50 | 530.70 | 83.62 | 111.28 |
| CTNNB1 | 245.10 | 481.80 | 83.14 | 101.03 |
| PBRM1 | 243.60 | 544.80 | 82.63 | 114.24 |
| NRas | 243.30 | 499.90 | 82.53 | 104.82 |
| BRCA2 | 238.20 | 525.60 | 80.80 | 110.21 |
| NOTCH1 | 236.10 | 544.30 | 80.09 | 114.13 |
| Ajuba | 233.90 | 483.60 | 79.34 | 101.40 |
| MAPK8 | 223.90 | 460.00 | 75.95 | 96.46 |
| FBXW7 | 203.20 | 432.60 | 68.93 | 90.71 |
| EGLN1 | 199.00 | 212.30 | 67.50 | 44.52 |
| RIPK1 | 182.50 | 464.00 | 61.91 | 97.30 |
| VHL | 32.50 | — | 11.02 | — |
| CREBBP | 31.10 | 568.00 | 10.55 | 119.10 |
| CHK1 | — | — | — | — |
| RBX1 | — | 509.50 | — | 106.84 |
| CUL3 | — | 469.30 | — | 98.41 |
| Weel | — | — | — | — |
| VHL | — | — | — | — |
| control | — | 13.40 | — | 2.81 |
| knockout | | | | |
| GFP | | | | |

—: no expression/no result

As can be seen by the CHK1 and WEE1 knockout, it is not predictable, if the modification of a gene predicted to be relevant for protein expression will result in a positive or negative effect, i.e. it can result in the opposite effect as intended, such as, stalling of cell division as in these two cases.

In the 4-day batch fermentation process, a productivity increase of at least 5% and up to 49% or even 99%, i.e. almost a doubling of productivity, for the MYC KO cell pools expressing different complex antibody formats compared to the unmodified cell pools or clones were obtained. It has to be pointed out that these cell pools comprise of a mixture of cells containing unedited, homozygous and heterozygous MYC loci. Thus, the improvement obtained with isolated clones will be even higher.

The results obtained for MYC gene inactivation were confirmed in a 14-day high cell density fed-batch cultivation (see Example 9). The result is shown in the following Table.

| | | | main peak | main peak |
|---|---|---|---|---|
| | IgG [mg/L]- | IgG [mg/L]- | (CE-SDS, %)- | (CE-SDS, %)- |
| | FAPcd137 | PD1-IL2v | FAPcd137 | PD1-IL2v |
| control, i.e. | 5200 | 5373 | 76 | 87 |
| w/o knockout | | | | |
| MYC gene | 7328 | 7440 | 70 | 85 |
| knockout | | | | |

The data with respect to the more than 80 knockouts tested shows the non-predictability of gene knockouts on productivity. As can be seen, only 40% of the knockouts resulted in a productivity increase, whereas the other rest showed no or a detrimental effect on cell growth or productivity. Some knockouts were lethal to the cell resulting in cell death or no/low cell proliferation.

The MYC gene knockout had the most profound effect on productivity in both cell lines. Sequencing of the PCR-amplified MYC locus within the MYC knockout cell pools revealed an abrupt interruption of the sequencing reaction at the first gRNA binding site. Without being bound by this theory, as a result, the expression of the encoded protein of the target gene is subsequently substantially reduced or disrupted in expression level.

The effect of the MYC knockout has been confirmed with various different protein as shown in the following Table.

| | control, i.e. | MYC gene | relative titer |
|---|---|---|---|
| | w/o knockout | knockout | increase |
| IgG [mg/L]- | 5200 | 7328 | 141% |
| FAPcd137 bispecific | | | |
| antibody | | | |
| IgG [mg/L]-PD1- | 5373 | 7440 | 138% |
| IL2v antibody-IL2v | | | |
| fusion | | | |
| IgG [mg/L]-PD1- | 4270 | 5633 | 132% |
| IL2v (2^{nd}) | | | |
| IgG [mg/L]-PD1- | 3527 | 5552 | 157% |
| IL2v (3^{rd}) | | | |
| IgG [mg/L]- | 7142 | 7640 | 107% |
| CD19CD28 | | | |
| bispecific antibody | | | |
| IgG [mg/L]- | 6375 | 7589 | 119% |
| CD19CD28 (2^{nd}) | | | |
| protein [mg/L]- | 6280 | 6635 | 106% |
| Dioxygenase | | | |
| protein [mg/L]- | 2107 | 2574 | 122% |
| bispecific Fab | | | |
| IgG [mg/L]- | 2118 | 2991 | 141% |
| CD20/TfR- | | | |
| bispecific brain | | | |
| shuttle format | | | |
| IgG [mg/L]-TCB- | 4868 | 5797 | 119% |
| T-cell bispecific | | | |
| antibody | | | |
| IgG [mg/L]- | | | |
| enzyme ligand-Fc | 3117 | 3636 | 117% |
| fusion | | | |
| bispecific antibody | 2405 | 3073 | 128% |
| average increase | | | 127% |

Beside the MYC gene knockout, the knockout of the genes STK11, SMAD4, PPP2CB, RBM38, NF1, CDK12, SIN3A, PARP-1, ATM, Hipk2, BARD1, HIF1AN, SMAD3, PALB2, FUBP1, RBL2, RPS6KA3, GPS2, ETS1, E2F5, CDKN1A, RNF43, EEF2K, AKT1, BRCA1, ATR, SMAD2, BAD, FOXO1, PBRM1, NRAS, BRCA2, NOTCH1, CREBBP, and RBX1 resulted in increased expression of the heterologous antibody.

Knockout of any one of the above listed gene activity/expression is advantageous in any eukaryotic cell used for the production of heterologous polypeptides, specifically in recombinant CHO cells used or intended to be used to produce recombinant polypeptides, especially antibodies, more specifically in targeted integration recombinant CHO cells. The knockout leads to a significant productivity increase. This is of high economic importance for any large-scale production process as this results in high yields of product obtainable from individual fed-batch cultivations.

The knockout of the genes of the above list is not limited to CHO cells but can also be used in other host cell lines, such as HEK293 cells, CAP cells, and BHK cells.

To knockout gene activity/expression CRISPR/Cas9 technology has been used. Likewise, any other technology can be employed such as Zinc-Finger-Nucleases or TALENS. In addition, RNA silencing species, such as siRNA/shRNA/miRNA can be employed to knockdown mRNA levels and as a consequence gene activity/expression.

Without being bound by this theory, it is assumed that a homozygous knockout has a more advantageous effect on productivity increase than a heterozygous knockout.

The invention is based, at least in part, on the finding that the functional knockout of at least one of the genes from the group of genes consisting of MYC, STK11, SMAD4, PPP2CB, RBM38, NF1, CDK12, SIN3A, PARP-1, ATM, Hipk2, BARD1, HIF1AN, SMAD3, PALB2, FUBP1, RBL2, RPS6KA3, GPS2, ETS1, E2F5, CDKN1A, RNF43, EEF2K, AKT1, BRCA1, ATR, SMAD2, BAD, FOXO1, PBRM1, NRAS, BRCA2, NOTCH1, CREBBP, and RBX1 in a mammalian cell, e.g. such as a CHO cell, improves recombinant productivity, especially of complex antibody formats.

The current invention is summarized below:

One independent aspect of the current invention is a mammalian cell wherein the activity or/and function or/and expression of at least one endogenous gene selected from the group of genes consisting of MYC, STK11, SMAD4, PPP2CB, RBM38, NF1, CDK12, SIN3A, PARP-1, ATM, Hipk2, BARD1, HIF1AN, SMAD3, PALB2, FUBP1, RBL2, RPS6KA3, GPS2, ETS1, E2F5, CDKN1A, RNF43, EEF2K, AKT1, BRCA1, ATR, SMAD2, BAD, FOXO1, PBRM1, NRAS, BRCA2, NOTCH1, CREBBP, and RBX1 has been reduced or eliminated or diminished or (completely) knocked-out.

One independent aspect of the current invention is a mammalian cell, wherein the expression of at least one endogenous gene selected from the group of genes consisting of MYC, STK11, SMAD4, PPP2CB, RBM38, NF1, CDK12, SIN3A, PARP-1, ATM, Hipk2, BARD1, HIF1AN, SMAD3, PALB2, FUBP1, RBL2, RPS6KA3, GPS2, ETS1, E2F5, CDKN1A, RNF43, EEF2K, AKT1, BRCA1, ATR, SMAD2, BAD, FOXO1, PBRM1, NRAS, BRCA2, NOTCH1, CREBBP, and RBX1 has been reduced, and wherein said mammalian has increased productivity for heterologous polypeptides compared to a cell that has the identical genotype but the respective endogenous gene expression of said at least one endogenous gene selected from the group of genes consisting of MYC, STK11, SMAD4, PPP2CB, RBM38, NF1, CDK12, SIN3A, PARP-1, ATM, Hipk2, BARD1, HIF1AN, SMAD3, PALB2, FUBP1, RBL2, RPS6KA3, GPS2, ETS1, E2F5, CDKN1A, RNF43, EEF2K, AKT1, BRCA1, ATR, SMAD2, BAD, FOXO1, PBRM1, NRAS, BRCA2, NOTCH1, CREBBP, and RBX1 and that is cultivated under the same conditions.

One independent aspect of the current invention is a method for increasing heterologous polypeptide titer of a recombinant mammalian cell, which has reduced expression of at least one endogenous gene selected from the group of genes consisting of MYC, STK11, SMAD4, PPP2CB, RBM38, NF1, CDK12, SIN3A, PARP-1, ATM, Hipk2, BARD1, HIF1AN, SMAD3, PALB2, FUBP1, RBL2, RPS6KA3, GPS2, ETS1, E2F5, CDKN1A, RNF43, EEF2K, AKT1, BRCA1, ATR, SMAD2, BAD, FOXO1, PBRM1, NRAS, BRCA2, NOTCH1, CREBBP, and RBX1, and which comprises an exogenous nucleic acid, i.e. a transgene, encoding said heterologous polypeptide compared to a cell cultivated under the same conditions that has the identical genotype but endogenous gene expression of said at least one endogenous gene selected from the group of genes consisting of MYC, STK11, SMAD4, PPP2CB, RBM38, NF1, CDK12, SIN3A, PARP-1, ATM, Hipk2, BARD1, HIF1AN, SMAD3, PALB2, FUBP1, RBL2, RPS6KA3, GPS2, ETS1, E2F5, CDKN1A, RNF43, EEF2K, AKT1, BRCA1, ATR, SMAD2, BAD, FOXO1, PBRM1, NRAS, BRCA2, NOTCH1, CREBBP, and RBX1.

One independent aspect of the current invention is a method for producing a recombinant mammalian cell with improved recombinant productivity, wherein the method comprises the following steps:
a) applying a nuclease-assisted and/or nucleic acid targeting at least one endogenous gene selected from the group of genes consisting of MYC, STK11, SMAD4, PPP2CB, RBM38, NF1, CDK12, SIN3A, PARP-1, ATM, Hipk2, BARD1, HIF1AN, SMAD3, PALB2, FUBP1, RBL2, RPS6KA3, GPS2, ETS1, E2F5, CDKN1A, RNF43, EEF2K, AKT1, BRCA1, ATR, SMAD2, BAD, FOXO1, PBRM1, NRAS, BRCA2, NOTCH1, CREBBP, and RBX1 in a mammalian cell to reduce the activity of said endogenous gene, and
b) selecting a mammalian cell wherein the activity of said endogenous gene has been reduced,
   thereby producing a recombinant mammalian cell with increased recombinant productivity compared to a cell cultivated under the same conditions that has the identical genotype but endogenous gene expression of said at least one endogenous gene selected from the group of genes consisting of MYC, STK11, SMAD4, PPP2CB, RBM38, NF1, CDK12, SIN3A, PARP-1, ATM, Hipk2, BARD1, HIF1AN, SMAD3, PALB2, FUBP1, RBL2, RPS6KA3, GPS2, ETS1, E2F5, CDKN1A, RNF43, EEF2K, AKT1, BRCA1, ATR, SMAD2, BAD, FOXO1, PBRM1, NRAS, BRCA2, NOTCH1, CREBBP, and RBX1.

One independent aspect of to the current invention is a method for producing a heterologous polypeptide comprising the steps of
a) cultivating a recombinant mammalian cell comprising an exogenous deoxyribonucleic acid encoding the heterologous polypeptide optionally under conditions suitable for the expression of the heterologous polypeptide, and
b) recovering the heterologous polypeptide from the cell or the cultivation medium,
wherein the activity or/and function or/and expression of at least one endogenous gene selected from the group of genes consisting of MYC, STK11, SMAD4, PPP2CB, RBM38, NF1, CDK12, SIN3A, PARP-1, ATM, Hipk2, BARD1, HIF1AN, SMAD3, PALB2, FUBP1, RBL2, RPS6KA3, GPS2, ETS1, E2F5, CDKN1A, RNF43, EEF2K, AKT1, BRCA1, ATR, SMAD2, BAD, FOXO1, PBRM1, NRAS, BRCA2, NOTCH1, CREBBP, and RBX1 has been reduced or eliminated or diminished or (completely) knocked-out in said mammalian cell.

Another independent aspect of the current invention is a method for producing a recombinant mammalian cell having/with improved and/or increased recombinant productivity, wherein the method comprises the following steps:
a) applying a nucleic acid or an enzyme or a nuclease-assisted gene targeting system targeting at least one endogenous gene selected from the group of genes consisting of MYC, STK11, SMAD4, PPP2CB, RBM38, NF1, CDK12, SIN3A, PARP-1, ATM, Hipk2, BARD1, HIF1AN, SMAD3, PALB2, FUBP1, RBL2, RPS6KA3, GPS2, ETS1, E2F5, CDKN1A, RNF43, EEF2K, AKT1, BRCA1, ATR, SMAD2, BAD, FOXO1, PBRM1, NRAS, BRCA2, NOTCH1, CREBBP, and RBX1 to a mammalian cell to reduce or eliminate or diminish or (completely) knockout the activity or/and function or/and expression of said endogenous gene, and
b) selecting a mammalian cell wherein the activity or/and function or/and expression of said endogenous gene has been reduced or eliminated or diminished or (completely) knocked-out,
thereby producing a recombinant mammalian cell having/with improved and/or increased recombinant productivity.

In one dependent embodiment of all aspects and embodiments of the current invention, the mammalian cell comprises a nucleic acid encoding a heterologous polypeptide.

In certain embodiments of all aspects and embodiments of the current invention, the nucleic acid encoding the heterologous polypeptide is operably linked to a promoter sequence functional is said mammalian cell and operably linked to a polyadenylation signal functional in said mammalian cell. In certain embodiments, the mammalian cell secretes the heterologous polypeptide when cultivated under suitable cultivation conditions.

In certain embodiments of all aspects and embodiments of the current invention, the knockout of at least one endogenous gene selected from the group of genes consisting of MYC, STK11, SMAD4, PPP2CB, RBM38, NF1, CDK12, SIN3A, PARP-1, ATM, Hipk2, BARD1, HIF1AN, SMAD3, PALB2, FUBP1, RBL2, RPS6KA3, GPS2, ETS1, E2F5, CDKN1A, RNF43, EEF2K, AKT1, BRCA1, ATR, SMAD2, BAD, FOXO1, PBRM1, NRAS, BRCA2, NOTCH1, CREBBP, and RBX1 is a heterozygous knockout or a homozygous knockout.

In certain embodiments of all aspects and embodiments of the current invention the productivity of the knockout cell line is at least 5%, preferably 10% or more, most preferred 20% or more increased compared to the respective mammalian cell with the same genotype but fully functional expression of said at least one endogenous gene selected from the group of genes consisting of MYC, STK11, SMAD4, PPP2CB, RBM38, NF1, CDK12, SIN3A, PARP-1, ATM, Hipk2, BARD1, HIF1AN, SMAD3, PALB2, FUBP1, RBL2, RPS6KA3, GPS2, ETS1, E2F5, CDKN1A, RNF43, EEF2K, AKT1, BRCA1, ATR, SMAD2, BAD, FOXO1, PBRM1, NRAS, BRCA2, NOTCH1, CREBBP, and RBX1.

In certain embodiments of all aspects and embodiments of the current invention, the reduction or elimination or diminishment or knockout of at least one endogenous gene selected from the group of genes consisting of MYC, STK11, SMAD4, PPP2CB, RBM38, NF1, CDK12, SIN3A, PARP-1, ATM, Hipk2, BARD1, HIF1AN, SMAD3, PALB2, FUBP1, RBL2, RPS6KA3, GPS2, ETS1, E2F5, CDKN1A, RNF43, EEF2K, AKT1, BRCA1, ATR, SMAD2, BAD, FOXO1, PBRM1, NRAS, BRCA2, NOTCH1, CREBBP, and RBX1 is mediated by a nuclease-assisted gene targeting system. In certain embodiments, the nuclease-assisted gene targeting system is selected from the group consisting of CRISPR/Cas9, CRISPR/Cpf1, zinc-finger nuclease and TALEN.

In certain embodiments of all aspects and embodiments of the current invention, the reduction of the expression of at least one endogenous gene selected from the group of genes consisting of MYC, STK11, SMAD4, PPP2CB, RBM38, NF1, CDK12, SIN3A, PARP-1, ATM, Hipk2, BARD1, HIF1AN, SMAD3, PALB2, FUBP1, RBL2, RPS6KA3, GPS2, ETS1, E2F5, CDKN1A, RNF43, EEF2K, AKT1, BRCA1, ATR, SMAD2, BAD, FOXO1, PBRM1, NRAS, BRCA2, NOTCH1, CREBBP, and RBX1 is mediated by RNA silencing. In certain embodiments, the RNA silencing is selected from the group consisting of siRNA gene targeting and knockdown, shRNA gene targeting and knockdown, and miRNA gene targeting and knockdown.

In certain embodiments of all aspects and embodiments of the current invention, the knockout of at least one endogenous gene selected from the group of genes consisting of MYC, STK11, SMAD4, PPP2CB, RBM38, NF1, CDK12, SIN3A, PARP-1, ATM, Hipk2, BARD1, HIF1AN, SMAD3, PALB2, FUBP1, RBL2, RPS6KA3, GPS2, ETS1, E2F5, CDKN1A, RNF43, EEF2K, AKT1, BRCA1, ATR, SMAD2, BAD, FOXO1, PBRM1, NRAS, BRCA2, NOTCH1, CREBBP, and RBX1 is performed i) before the introduction of the exogenous nucleic acid encoding the heterologous polypeptide, or ii) after the introduction of the exogenous nucleic acid encoding the heterologous polypeptide.

In certain embodiments of all aspects and embodiments of the current invention, the heterologous polypeptide is an antibody. In certain embodiments, the antibody is an antibody comprising two or more different binding sites and optionally a domain exchange. In certain embodiments, the antibody comprises three or more binding sites or VH/VL-pairs or Fab fragments and optionally a domain exchange. In certain embodiments, the antibody is a multispecific antibody.

In certain embodiments of all aspects and embodiments of the current invention, the heterologous polypeptide is selected from the group of heterologous polypeptides comprising multispecific antibodies and antibody-multimer-fusion polypeptide. In certain embodiments, the heterologous polypeptide is selected from the group consisting of
i) a full-length antibody with domain exchange comprising a first Fab fragment and a second Fab fragment,
   wherein in the first Fab fragment
      a) the light chain of the first Fab fragment comprises a VL and a CH1 domain and the heavy chain of the first Fab fragment comprises a VH and a CL domain;
      b) the light chain of the first Fab fragment comprises a VH and a CL domain and the heavy chain of the first Fab fragment comprises a VL and a CH1 domain; or
      c) the light chain of the first Fab fragment comprises a VH and a CH1 domain and the heavy chain of the first Fab fragment comprises a VL and a CL domain;
   and
   wherein the second Fab fragment comprises a light chain comprising a VL and a CL domain, and a heavy chain comprising a VH and a CH1 domain;
ii) a full-length antibody with domain exchange and additional heavy chain C-terminal binding site comprising
   one full length antibody comprising two pairs each of a full length antibody light chain and a full length antibody heavy chain, wherein the binding sites formed by each of the pairs of the full length heavy chain and the full length light chain specifically bind to a first antigen;
      and
   one additional Fab fragment, wherein the additional Fab fragment is fused to the C-terminus of one heavy chain of the full length antibody, wherein the binding site of the additional Fab fragment specifically binds to a second antigen;
   wherein the additional Fab fragment specifically binding to the second antigen i) comprises a domain crossover such that a) the light chain variable domain (VL) and the heavy chain variable domain (VH) are replaced by each other, or b) the light chain constant domain (CL) and the heavy chain constant domain (CH1) are replaced by each other, or ii) is a single chain Fab fragment;
iii) a one-armed single chain antibody comprising a first binding site that specifically binds to a first epitope or antigen and a second binding site that specifically binds to a second epitope or antigen, comprising
   a light chain comprising a variable light chain domain and a light chain kappa or lambda constant domain;
   a combined light/heavy chain comprising a variable light chain domain, a light chain constant domain, a peptidic linker, a variable heavy chain domain, a CH1 domain, a Hinge region, a CH2 domain, and a CH3 with knob mutation;
   a heavy chain comprising a variable heavy chain domain, a CH1 domain, a hinge region, a CH2 domain, and a CH3 domain with hole mutation;
iv) a two-armed single chain antibody comprising a first binding site that specifically binds to a first epitope or antigen and a second binding site that specifically binds to a second epitope or antigen, comprising
   a first combined light/heavy chain comprising a variable light chain domain, a light chain constant domain, a peptidic linker, a variable heavy chain domain, a CH1 domain, a Hinge region, a CH2 domain, and a CH3 with hole mutation;
   a second combined light/heavy chain comprising a variable light chain domain, a light chain constant domain, a peptidic linker, a variable heavy chain domain, a CH1 domain, a Hinge region, a CH2 domain, and a CH3 domain with knob mutation;
v) a common light chain bispecific antibody comprising a first binding site that specifically binds to a first epitope or antigen and a second binding site that specifically binds to a second epitope or antigen, comprising
   a light chain comprising a variable light chain domain and a light chain constant domain;
   a first heavy chain comprising a variable heavy chain domain, a CH1 domain, a Hinge region, a CH2 domain, and a CH3 domain with hole mutation;
   a second heavy chain comprising a variable heavy chain domain, a CH1 domain, a Hinge region, a CH2 domain, and a CH3 domain with knob mutation;
vi) a full-length antibody with additional heavy chain N-terminal binding site with domain exchange comprising
   a first and a second Fab fragment, wherein each binding site of the first and the second Fab fragment specifically bind to a first antigen;
   a third Fab fragment, wherein the binding site of the third Fab fragment specifically binds to a second antigen, and wherein the third Fab fragment comprises a domain crossover such that the variable light chain domain (VL) and the variable heavy chain domain (VH) are replaced by each other; and
   an Fc-region comprising a first Fc-region polypeptide and a second Fc-region polypeptide;
   wherein the first and the second Fab fragment each comprise a heavy chain fragment and a full-length light chain,
   wherein the C-terminus of the heavy chain fragment of the first Fab fragment is fused to the N-terminus of the first Fc-region polypeptide,
   wherein the C-terminus of the heavy chain fragment of the second Fab fragment is fused to the N-terminus of the variable light chain domain of the third Fab fragment and the C-terminus of the CH1 domain of the third Fab fragment is fused to the N-terminus of the second Fc-region polypeptide;
vii) an immunoconjugate comprising a full-length antibody and a non-immunoglobulin moiety conjugated to each other optionally via a peptidic linker,
and
viii) an antibody-multimer-fusion polypeptide comprising
   (a) an antibody heavy chain and an antibody light chain, and
   (b) a first fusion polypeptide comprising in N- to C-terminal direction a first part of a non-antibody multimeric polypeptide, an antibody heavy chain CH1 domain or an antibody light chain constant domain, an antibody hinge region, an antibody heavy chain CH2 domain and an antibody heavy chain CH3 domain, and a second fusion polypeptide comprising in N- to C-terminal direction the second part of the non-antibody multimeric polypeptide and an antibody light chain constant domain if the first polypeptide comprises an antibody heavy chain CH1 domain or an antibody heavy chain CH1 domain if the first polypeptide comprises an antibody light chain constant domain,
   wherein
      (i) the antibody heavy chain of (a) and the first fusion polypeptide of (b), (ii) the antibody heavy chain of (a) and the antibody light chain of (a), and (iii) the first fusion polypeptide of (b) and the second fusion polypeptide of (b) are each independently of each other covalently linked to each other by at least one disulfide bond,
   wherein
      the variable domains of the antibody heavy chain and the antibody light chain form a binding site specifically binding to an antigen.

In certain embodiments of all aspects and embodiments of the current invention, the at least one endogenous gene is selected from the group of genes consisting of MYC, STK11, SMAD4, PPP2CB, RBM38, CDK12, PARP-1, ATM, Hipk2, BARD1, and SMAD3. In certain embodiments, the at least one endogenous gene is selected from the group of genes consisting of MYC, STK11, SMAD4, PPP2CB, RBM38, and CDK12. In certain embodiments, the at least one endogenous gene is selected from the group of genes consisting of MYC and STK11. In one preferred embodiment the at least one endogenous gene is MYC.

In certain embodiments of all aspects and embodiments of the current invention, the heterologous polypeptide is an antibody-multimer-fusion polypeptide comprising
(a) an antibody heavy chain and an antibody light chain, and
(b) a first fusion polypeptide comprising in N- to C-terminal direction a first part of a non-antibody multimeric polypeptide, an antibody heavy chain CH1 domain or an antibody light chain constant domain, an antibody hinge region, an antibody heavy chain CH2 domain and an antibody heavy chain CH3 domain, and a second fusion polypeptide comprising in N- to C-terminal direction the second part of the non-antibody multimeric polypeptide and an antibody light chain constant domain if the first polypeptide comprises an antibody heavy chain CH1 domain or an antibody heavy chain CH1 domain if the first polypeptide comprises an antibody light chain constant domain,
wherein
   (i) the antibody heavy chain of (a) and the first fusion polypeptide of (b), (ii) the antibody heavy chain of (a) and the antibody light chain of (a), and (iii) the first fusion polypeptide of (b) and the second fusion polypeptide of (b) are each independently of each other covalently linked to each other by at least one disulfide bond,
wherein
   the variable domains of the antibody heavy chain and the antibody light chain form a binding site specifically binding to an antigen.

In certain embodiments, the at least one endogenous gene is selected from the group of genes consisting of MYC, STK11, SMAD4, PPP2CB, RBM38, NF1, CDK12, SIN3A, PARP-1, ATM, Hipk2, BARD1, HIF1AN, SMAD3, and CDKN1A. In certain embodiments, the at least one endogenous gene is selected from the group of genes consisting of MYC, STK11, SMAD4, PPP2CB, RBM38, NF1, and CDK12. In certain embodiments, the at least one endogenous gene is selected from the group of genes consisting of MYC, STK11, SMAD4, and PPP2CB. In certain embodiments, the at least one endogenous gene is MYC. In certain embodiments, the first fusion polypeptide comprises as first part of the non-antibody multimeric polypeptide two ectodomains of a TNF ligand family member or a fragment thereof that are connected to each other by a peptide linker, and the second fusion polypeptide comprises as second part of a non-antibody multimeric polypeptide only one ectodomain of said TNF ligand family member or a fragment thereof, or vice versa. In certain embodiments, the first fusion polypeptide comprises in N- to C-terminal direction a first part of a non-antibody multimeric polypeptide, an antibody light chain constant domain, an antibody hinge region, an antibody heavy chain CH2 domain and an antibody heavy chain CH3 domain, and the second fusion polypeptide comprising in N- to C-terminal direction the second part of the non-antibody multimeric polypeptide and an antibody heavy chain CH1 domain. In certain embodiments, in the CL domain adjacent to the part of a non-antibody multimeric polypeptide the amino acid at position 123 (Kabat EU numbering) has been replaced by arginine (R) and the amino acid at position 124 (Kabat EU numbering) has been substituted by lysine (K), and wherein in the CH1 domain adjacent to the part of a non-antibody multimeric polypeptide the amino acids at position 147 (Kabat EU numbering) and at position 213 (Kabat EU numbering) have been substituted by glutamic acid (E). In certain embodiments, the variable domains of the antibody heavy chain and the antibody light chain form a binding site specifically binding to a cell surface antigen selected from the group consisting of Fibroblast Activation Protein (FAP), Melanoma-associated Chondroitin Sulfate Proteoglycan (MCSP), Epidermal Growth Factor Receptor (EGFR), Carcinoembryonic Antigen (CEA), CD19, CD20 and CD33. In certain embodiments, the TNF ligand family member co-stimulates human T-cell activation. In certain embodiments, the TNF ligand family member is selected from 4-1BBL and OX40L. In certain embodiments, the TNF ligand family member is 4-1BBL and the cell surface antigen is FAP.

In certain embodiments of all aspects and embodiments of the current invention, the heterologous polypeptide is a fusion polypeptide comprising a bivalent, mono- or bispecific full-length antibody and a non-immunoglobulin moiety, wherein the antibody is conjugated to the non-immunoglobulin moiety at a single terminus of one of the heavy or light chains of the antibody optionally via a peptidic linker. In certain embodiments, the at least one endogenous gene is selected from the group of genes consisting of MYC, STK11, SMAD4, PPP2CB, RBM38, CDK12, PARP-1, ATM, Hipk2, BARD1, SMAD3, PALB2, FUBP1, RBL2, RPS6KA3, GPS2, ETS1, E2F5, CDKN1A, RNF43, EEF2K, AKT1, BRCA1, ATR, SMAD2, BAD, FOXO1, PBRM1, NRAS, BRCA2, NOTCH1, CREBBP, and RBX1. In certain embodiments, the at least one endogenous gene is selected from the group of genes consisting of MYC, STK11, SMAD4, PPP2CB, RBM38, CDK12, PARP-1, BARD1, ETS1, E2F5, RNF43, EEF2K, AKT1, BRCA1, BAD, FOXO1, PBRM1, BRCA2, NOTCH1, and CREBBP. In certain embodiments, the at least one endogenous gene is selected from the group of genes consisting of MYC, STK11, and CDK12. In certain embodiments, the at least one endogenous gene is MYC. In certain embodiments, the heterologous polypeptide is an anti-PD-1 antibody conjugated to interleukin 2. In certain embodiments, the interleukin-2 is an engineered IL2v moiety with abolished binding to IL-2Ra (CD25) to avoid undesired CD25-mediated toxicities and Treg expansion.

In certain embodiments of all aspects and embodiments of the invention, the mammalian cell is a CHO cell or a HEK cell. In certain embodiments, the mammalian cell is a CHO-K1 cell. In certain embodiments, the mammalian cell is a suspension growing mammalian cell.

Another independent aspect of the current invention is a method for producing a recombinant mammalian cell comprising a deoxyribonucleic acid encoding a heterologous polypeptide and secreting the heterologous polypeptide comprising the following steps:
a) providing a mammalian cell comprising an exogenous nucleotide sequence integrated at a single site within a locus of the genome of the mammalian cell, wherein the exogenous nucleotide sequence comprises a first and a second recombination recognition sequence flanking at least one first selection marker, and a third recombination recognition sequence located between the first and the second recombination recognition sequence, and all the recombination recognition sequences are different, wherein the activity/expression/function of at least one endogenous gene selected from the group of genes consisting of MYC, STK11, SMAD4, PPP2CB, RBM38, NF1, CDK12, SIN3A, PARP-1, ATM, Hipk2, BARD1, HIF1AN, SMAD3, PALB2, FUBP1, RBL2, RPS6KA3, GPS2, ETS1, E2F5, CDKN1A, RNF43, EEF2K, AKT1, BRCA1, ATR, SMAD2, BAD, FOXO1, PBRM1, NRAS, BRCA2, NOTCH1, CREBBP, and RBX1 is reduced/eliminated/knocked-out;
b) introducing into the cell provided in a) a composition of two deoxyribonucleic acids comprising three different recombination recognition sequences and one to eight expression cassettes for polypeptides of the heterologous polypeptide,
   wherein
      the first deoxyribonucleic acid comprises in 5′- to 3′-direction,
         a first recombination recognition sequence,
         one or more expression cassette(s),
         a 5′-terminal part of an expression cassette encoding one second selection marker, and
         a first copy of a third recombination recognition sequence,
      and
      the second deoxyribonucleic acid comprises in 5′- to 3′-direction
         a second copy of the third recombination recognition sequence,
         a 3′-terminal part of an expression cassette encoding the one second selection marker,
         one or more expression cassette(s), and
         a second recombination recognition sequence,
   wherein the first to third recombination recognition sequences of the first and second deoxyribonucleic acids are matching the first to third recombination recognition sequence on the integrated exogenous nucleotide sequence,
   wherein the 5′-terminal part and the 3′-terminal part of the expression cassette encoding the one second selection marker when taken together form a functional expression cassette of the one second selection marker;
c) introducing
   i) either simultaneously with the first and second deoxyribonucleic acid of b);
      or
   ii) sequentially thereafter
   one or more recombinase,
   wherein the one or more recombinases recognize the recombination recognition sequences of the first and the second deoxyribonucleic acid; (and optionally wherein the one or more recombinases perform two recombinase mediated cassette exchanges)
and
d) selecting for cells expressing the second selection marker and secreting the heterologous polypeptide,
thereby producing a recombinant mammalian cell comprising a deoxyribonucleic acid encoding the heterologous polypeptide and secreting the heterologous polypeptide.

Another independent aspect of the current invention is a method for producing a recombinant mammalian cell comprising a deoxyribonucleic acid encoding a heterologous polypeptide and secreting the heterologous polypeptide comprising the following steps:
a) providing a mammalian cell comprising an exogenous nucleotide sequence integrated at a single site within a locus of the genome of the mammalian cell, wherein the exogenous nucleotide sequence comprises a first and a second recombination recognition sequence flanking at least one first selection marker, and a third recombination recognition sequence located between the first and the second recombination recognition sequence, and all the recombination recognition sequences are different;
b) introducing into the cell provided in a) a composition of two deoxyribonucleic acids comprising three different recombination recognition sequences and one to eight expression cassettes for polypeptides of the heterologous polypeptide,
   wherein
      the first deoxyribonucleic acid comprises in 5′- to 3′-direction,
         a first recombination recognition sequence,
         one or more expression cassette(s),
         a 5′-terminal part of an expression cassette encoding one second selection marker, and
         a first copy of a third recombination recognition sequence,
      and
      the second deoxyribonucleic acid comprises in 5′- to 3′-direction
         a second copy of the third recombination recognition sequence,
         a 3′-terminal part of an expression cassette encoding the one second selection marker,
         one or more expression cassette(s), and
         a second recombination recognition sequence,
   wherein the first to third recombination recognition sequences of the first and second deoxyribonucleic acids are matching the first to third recombination recognition sequence on the integrated exogenous nucleotide sequence,
   wherein the 5′-terminal part and the 3′-terminal part of the expression cassette encoding the one second selection marker when taken together form a functional expression cassette of the one second selection marker;
c) introducing
   i) either simultaneously with the first and second deoxyribonucleic acid of b);
      or
   ii) sequentially thereafter
   one or more recombinase,
   wherein the one or more recombinases recognize the recombination recognition sequences of the first and the second deoxyribonucleic acid; (and optionally wherein the one or more recombinases perform two recombinase mediated cassette exchanges)
d) optionally selecting for cells expressing the second selection marker and secreting the heterologous polypeptide,
e) reducing/eliminating/knocking-out the activity/expression/function of at least one endogenous gene selected from the group of genes consisting of MYC, STK11, SMAD4, PPP2CB, RBM38, NF1, CDK12, SIN3A, PARP-1, ATM, Hipk2, BARD1, HIF1AN, SMAD3, PALB2, FUBP1, RBL2, RPS6KA3, GPS2, ETS1, E2F5, CDKN1A, RNF43, EEF2K, AKT1, BRCA1, ATR, SMAD2, BAD, FOXO1, PBRM1, NRAS, BRCA2, NOTCH1, CREBBP, and RBX1;
and
f) selecting for cells expressing and secreting the heterologous polypeptide, optionally with higher titer than those of step d),
thereby producing a recombinant mammalian cell comprising a deoxyribonucleic acid encoding the heterologous polypeptide and secreting the heterologous polypeptide.

In certain embodiments of all aspects and embodiments of the current invention, the recombinase is Cre recombinase.

In certain embodiments of all aspects and embodiments of the current invention, the deoxyribonucleic acid is stably integrated into the genome of the mammalian cell at a single site or locus.

In certain embodiments of all aspects and embodiments of the current invention, the deoxyribonucleic acid encoding the heterologous polypeptide comprises at least 4 expression cassettes wherein
a first recombination recognition sequence is located 5′ to the most 5′ (i.e. first) expression cassette,
a second recombination recognition sequence is located 3′ to the most 3′ expression cassette, and
a third recombination recognition sequence is located
between the first and the second recombination recognition sequence, and
between two of the expression cassettes,
and
wherein all recombination recognition sequences are different.

In certain embodiments of all aspects and embodiments of the current invention, the third recombination recognition sequence is located between the fourth and the fifth expression cassette.

In certain embodiments of all aspects and embodiments of the current invention, the deoxyribonucleic acid encoding the heterologous polypeptide comprises a further expression cassette encoding for a selection marker.

In certain embodiments of all aspects and embodiments of the current invention, the deoxyribonucleic acid encoding the heterologous polypeptide comprises a further expression cassette encoding for a selection marker, and the expression cassette encoding for the selection marker is located partly 5′ and partly 3′ to the third recombination recognition sequence, wherein the 5′-located part of said expression cassette comprises the promoter and the start-codon and the 3′-located part of said expression cassette comprises the coding sequence without a start-codon and a polyA signal, wherein the start-codon is operably linked to the coding sequence.

In certain embodiments of all aspects and embodiments of the current invention, the expression cassette encoding for a selection marker is located either
i) 5′, or
ii) 3′, or
iii) partly 5′ and partly 3′
to the third recombination recognition sequence.

In certain embodiments of all aspects and embodiments of the current invention, the expression cassette encoding for a selection marker is located partly 5′ and partly 3′ to the third recombination recognition sequences, wherein the 5′-located part of said expression cassette comprises the promoter and a start-codon and the 3′-located part of said expression cassette comprises the coding sequence without a start-codon and a polyA signal.

In certain embodiments of all aspects and embodiments of the current invention, the 5′-located part of the expression cassette encoding the selection marker comprises a promoter sequence operably linked to a start-codon, whereby the promoter sequence is flanked upstream by (i.e. is positioned downstream to) the second, third or fourth, respectively, expression cassette and the start-codon is flanked downstream by (i.e. is positioned upstream of) the third recombination recognition sequence; and the 3′-located part of the expression cassette encoding the selection marker comprises a nucleic acid encoding the selection marker lacking a start-codon and is flanked upstream by the third recombination recognition sequence and downstream by the third, fourth or fifth, respectively, expression cassette.

In certain embodiments of all aspects and embodiments of the current invention, the start-codon is a translation start-codon. In certain embodiments, the start-codon is ATG.

In certain embodiments of all aspects and embodiments of the current invention, the first deoxyribonucleic acid is integrated into a first vector and the second deoxyribonucleic acid is integrated into a second vector.

In certain embodiments of all aspects and embodiments of the current invention, each of the expression cassettes comprise in 5′-to-3′ direction a promoter, a coding sequence and a polyadenylation signal sequence optionally followed by a terminator sequence.

In certain embodiments of all aspects and embodiments of the current invention, the heterologous polypeptide is selected from the group of polypeptides consisting of a bivalent, monospecific antibody, a bivalent, bispecific antibody comprising at least one domain exchange, and a trivalent, bispecific antibody comprising at least one domain exchange.

In certain embodiments of all aspects and embodiments of the current invention, the recombinase recognition sequences are L3, 2L and LoxFas. In certain embodiments, L3 has the sequence of SEQ ID NO: 01, 2L has the sequence of SEQ ID NO: 02 and LoxFas has the sequence of SEQ ID NO: 03. In certain embodiments, the first recombinase recognition sequence is L3, the second recombinase recognition sequence is 2L and the third recombinase recognition sequence is LoxFas.

In certain embodiments of all aspects and embodiments of the current invention, the promoter is the human CMV promoter with intron A, the polyadenylation signal sequence is the bGH polyA site and the terminator sequence is the hGT terminator.

In certain embodiments of all aspects and embodiments of the current invention, the promoter is the human CMV promoter with intron A, the polyadenylation signal sequence is the bGH polyA site and the terminator sequence is the hGT terminator except for the expression cassette(s) of the selection marker(s), wherein the promoter is the SV40 promoter and the polyadenylation signal sequence is the SV40 polyA site and a terminator sequence is absent.

In certain embodiments of all aspects and embodiments of the current invention, the human CMV promoter has the sequence of SEQ ID NO: 04. In certain embodiments, the human CMV promoter has the sequence of SEQ ID NO: 05. In certain embodiments, the human CMV promoter has the sequence of SEQ ID NO: 06.

In certain embodiments of all aspects and embodiments of the current invention, the SV40 polyadenylation signal sequence is SEQ ID NO: 07.

In certain embodiments of all aspects and embodiments of the current invention, the bGH polyadenylation signal sequence is SEQ ID NO: 08.

In certain embodiments of all aspects and embodiments of the current invention, the hGT terminator has the sequence of SEQ ID NO: 09.

In certain embodiments, of all aspects and embodiments of the current invention, the SV40 promoter has the sequence of SEQ ID NO: 10.

The following examples, figures and sequences are provided to aid the understanding of the present invention, the true scope of which is set forth in the appended claims. It is understood that modifications can be made in the procedures set forth without departing from the spirit of the invention.

### DESCRIPTION OF THE FIGURES

All Figures: Verification of knockout obtained with three independent gRNAs for exemplary genes within a CHO cell. Chromatogram of DNA sequences spanning the deletion regions are given for the unmodified parent cell (upper chromatogram; denoted as “1.WT.ab1”) and the cell with the knockout (lower chromatogram; denoted as “2.KO.ab1”) obtained 7 days after RNP nucleofection. Sanger sequencing was performed to validate the location and nature of the insertion and deletion events. The location of gRNA and PAM motif are indicated for each gRNA sequence, respectively. The cleavage site of the respective guide is indicated by a vertical line. Genomic Location in published CHO genome: (PICR Genome); see https://www.ncbi. nlm.nih.gov/assembly/GCF_003668045.3/.

FIG. 1: MYC knockout Sanger sequencing result; Genomic Location: RAZU01000002.1: 8,114,040-8,118,048.

FIG. 2: STK11 knockout Sanger sequencing result; Genomic Location: RAZU01000219.1 (10540304..10556926).

FIG. 3: PPP2CB knockout Sanger sequencing result; Genomic Location: RAZU01000044.1 (18735335..18754967).

FIG. 4: RBM38 knockout Sanger sequencing result; Genomic Location RAZU01000236.1 (501782..514198).

FIG. 5: NF1 knockout Sanger sequencing result; Genomic Location: RAZU01001831.1 (12672140..12907880).

FIG. 6: CDK12 knockout Sanger sequencing result; Genomic Location: RAZU01000239.1 (886868..960350).

FIG. 7: SIN3A knockout Sanger sequencing result; Genomic Location: RAZU01000166.1 (7107379..7169085).

FIG. 8: PARP-1 knockout Sanger sequencing result; Genomic Location: RAZU01000210.1 (10105791..10139187).

FIG. 9: ATM knockout Sanger sequencing result; Genomic Location: RAZU01000166.1 (10510024..10617455).

FIG. 10: Hipk2 knockout Sanger sequencing result; Genomic Location: RAZU01000045.1 (12630354..12820847).

FIG. 11: BARD1 knockout Sanger sequencing result; Genomic Location: RAZU01000074.1 (44502103..44567572).

FIG. 12: SMAD3 knockout Sanger sequencing result; Genomic Location: RAZU01000166.1 (480665..597614).

FIG. 13: CDKN1A knockout Sanger sequencing result; Genomic Location: RAZU01000063.1 (8736827..8768979).

### DESCRIPTION OF THE SEQUENCES

SEQ ID NO: 01: exemplary sequence of an L3 recombinase recognition sequence
SEQ ID NO: 02: exemplary sequence of a 2L recombinase recognition sequence
SEQ ID NO: 03: exemplary sequence of a LoxFas recombinase recognition sequence
SEQ ID NO: 04-06: exemplary variants of human CMV promoter
SEQ ID NO: 07: exemplary SV40 polyadenylation signal sequence
SEQ ID NO: 08: exemplary bGH polyadenylation signal sequence
SEQ ID NO: 09: exemplary hGT terminator sequence
SEQ ID NO: 10: exemplary SV40 promoter sequence
SEQ ID NO: 11: exemplary GFP nucleic acid sequence
SEQ ID NO: 12-14: SIRT-1 guide RNAs
SEQ ID NO: 15-17: MYC guide RNAs
SEQ ID NO: 18-20: STK11 guide RNAs
SEQ ID NO: 21-23: SMAD4 guide RNAs
SEQ ID NO: 24-26: PPP2CB guide RNAs
SEQ ID NO: 27-29: RBM38 guide RNAs
SEQ ID NO: 30-32: NF1 guide RNAs
SEQ ID NO: 33-35: CDK12 guide RNAs
SEQ ID NO: 36-38: SIN3A guide RNAs
SEQ ID NO: 39-41: PARP-1 guide RNAs
SEQ ID NO: 42-44: ATM guide RNAs
SEQ ID NO: 45-47: Hipk2 guide RNAs
SEQ ID NO: 48-50: BARD1 guide RNAs
SEQ ID NO: 51-53: HIF1AN guide RNAs
SEQ ID NO: 54-56: SMAD3 guide RNAs
SEQ ID NO: 57-59: CDKN1A guide RNAs
SEQ ID NO: 60+61: MYC verification primer forward and reverse
SEQ ID NO: 62+63: STK11 verification primer forward and reverse
SEQ ID NO: 64+65: SMAD4 verification primer forward and reverse
SEQ ID NO: 66+67: PPP2CB verification primer forward and reverse
SEQ ID NO: 68+69: RBM38 verification primer forward and reverse
SEQ ID NO: 70+71: NF1 verification primer forward and reverse
SEQ ID NO: 72+73: CDK12 verification primer forward and reverse
SEQ ID NO: 74+75: SIN3A verification primer forward and reverse
SEQ ID NO: 76+77: PARP-1 verification primer forward and reverse
SEQ ID NO: 78+79: ATM verification primer forward and reverse
SEQ ID NO: 80+81: Hipk2 verification primer forward and reverse
SEQ ID NO: 82+83: BARD1 verification primer forward and reverse
SEQ ID NO: 84+85: HIF1AN verification primer forward and reverse
SEQ ID NO: 86+87: SMAD3 verification primer forward and reverse
SEQ ID NO: 88+89: CDKN1A verification primer forward and reverse.

### EXAMPLES

### Example 1

### General Techniques

### 1) Recombinant DNA Techniques

Standard methods were used to manipulate DNA as described in Sambrook et al., Molecular Cloning: A Laboratory Manual, Second Edition, Cold Spring Harbor Laboratory Press, Cold Spring Harbor, N.Y, (1989). The molecular biological reagents were used according to the manufacturer's instructions.

### 2) DNA Sequence Determination

DNA sequencing was performed at SequiServe GmbH (Vaterstetten, Germany) or Eurofins Genomics GmbH (Ebersberg, Germany) or Microsynth AG (Balgach, Switzerland).

### 3) DNA and Protein Sequence Analysis and Sequence Data Management

The EMBOSS (European Molecular Biology Open Software Suite) software package and Geneious prime 2019 (Auckland, New Zealand) were used for sequence creation, mapping, analysis, annotation and illustration.

### 4) Gene and Oligonucleotide Synthesis

Desired gene segments were prepared by chemical synthesis at Geneart GmbH (Regensburg, Germany) or Twist Bioscience (San Francisco, USA). The synthesized gene fragments were cloned into an E. coli plasmid for propagation/amplification. The DNA sequences of subcloned gene fragments were verified by DNA sequencing. Alternatively, short synthetic DNA fragments were assembled by annealing chemically synthesized oligonucleotides or via PCR. The respective oligonucleotides were prepared by metabion GmbH (Planegg-Martinsried, Germany).

### 5) Reagents

All commercial chemicals, antibodies and kits were used as provided according to the manufacturer's protocol if not stated otherwise.

### 6) Cultivation of TI Host Cell Line

TI CHO host cells were cultivated at 37° C. in a humidified incubator with 85% humidity and 5% CO₂. They were cultivated in a proprietary DMEM/F12-based medium containing 300 ng/ml Hygromycin B and 4 μg/ml of a second selection marker. The cells were splitted every 3 or 4 days at a concentration of 0.3×10E6 cells/ml in a total volume of 30 ml. For the cultivation 125 ml non-baffle Erlenmeyer shake flasks were used. Cells were shaken at 150 rpm with a shaking amplitude of 5 cm. The cell count was determined with Cedex HiRes Cell Counter (Roche). Cells were kept in culture until they reached an age of 60 days.

### 7) Cloning

### General:

Cloning with R-sites depends on DNA sequences next to the gene of interest (GOI) that are equal to sequences lying in following fragments. Like that, assembly of fragments is possible by overlap of the equal sequences and subsequent sealing of nicks in the assembled DNA by a DNA ligase. Therefore, a cloning of the single genes in particular preliminary vectors containing the right R-sites is necessary. After successful cloning of these preliminary vectors the gene of interest flanked by the R-sites is cut out via restriction digest by enzymes cutting directly next to the R-sites. The last step is the assembly of all DNA fragments in one-step. In more detail, a 5′-exonuclease removes the 5′-end of the overlapping regions (R-sites). After that, annealing of the R-sites can take place and a DNA polymerase extends the 3′-end to fill the gaps in the sequence. Finally, the DNA ligase seals the nicks in between the nucleotides. Addition of an assembly master mix containing different enzymes like exonucleases, DNA polymerases and ligases, and subsequent incubation of the reaction mix at 50° C. leads to an assembly of the single fragments to one plasmid. After that, competent E. coli cells are transformed with the plasmid.

For some vectors, a cloning strategy via restriction enzymes was used. By selection of suitable restriction enzymes, the wanted gene of interest can be cut out and afterwards inserted into a different vector by ligation. Therefore, enzymes cutting in a multiple cloning site (MCS) are preferably used and chosen in a smart manner, so that a ligation of the fragments in the correct array can be conducted. If vector and fragment are previously cut with the same restriction enzyme, the sticky ends of fragment and vector fit perfectly together and can be ligated by a DNA ligase, subsequently. After ligation, competent E. coli cells are transformed with the newly generated plasmid.

### Cloning Via Restriction Digestion:

For the digest of plasmids with restriction enzymes, the following components were pipetted together on ice:

TABLE
Restriction Digestion Reaction Mix

| component | ng (set point) | μl |
|---|---|---|
| purified DNA | tbd | tbd |
| CutSmart Buffer (10×) | | 5 |
| Restriction Enzyme | | 1 |
| PCR-grade Water | | ad 50 |
| Total | | 50 |

If more enzymes were used in one digestion, 1 μl of each enzyme was used and the volume adjusted by addition of more or less PCR-grade water. All enzymes were selected on the preconditions that they are qualified for the use with CutSmart buffer from new England Biolabs (100% activity) and have the same incubation temperature (all 37° C.).

Incubation was performed using thermomixers or thermal cyclers, allowing incubating the samples at a constant temperature (37° C.). During incubation the samples were not agitated. Incubation time was set at 60 min. Afterwards the samples were directly mixed with loading dye and loaded onto an agarose electrophoresis gel or stored at 4° C./on ice for further use.

A 1 agarose gel was prepared for gel electrophoresis. Therefor 1.5 g of multi-purpose agarose were weighed into a 125 Erlenmeyer shake flask and filled up with 150 ml TAE-buffer. The mixture was heated up in a microwave oven until the agarose was completely dissolved. 0.5 μg/ml ethidium bromide were added into the agarose solution. Thereafter the gel was cast in a mold. After the agarose was set, the mold was placed into the electrophoresis chamber and the chamber filled with TAE-buffer. Afterwards the samples were loaded. In the first pocket (from the left) an appropriate DNA molecular weight marker was loaded, followed by the samples. The gel was run for around 60 minutes at <130 V. After electrophoresis, the gel was removed from the chamber and analyzed in an UV-Imager.

The target bands were cut and transferred to 1.5 ml Eppendorf tubes. For purification of the gel, the QIAquick Gel Extraction Kit from Qiagen was used according to the manufacturer's instructions. The DNA fragments were stored at −20° C. for further use.

The fragments for the ligation were pipetted together in a molar ratio of 1:2, 1:3 or 1:5 vector to insert, depending on the length of the inserts and the vector-fragments and their correlation to each other. If the fragment, that should be inserted into the vector was short, a 1:5-ratio was used. If the insert was longer, a smaller amount of it was used in correlation to the vector. An amount of 50 ng of vector were used in each ligation and the particular amount of insert calculated with NEBioCalculator. For ligation, the T4 DNA ligation kit from NEB was used. An example for the ligation mixture is depicted in the following Table:

TABLE
Ligation Reaction Mix

| component | ng (set point) | conc. [ng/μl] | μl |
|---|---|---|---|
| T4 DNA Ligase Buffer (10×) | | | 2 |
| Vector DNA (4000 bp) | 50 | 50 | 1 |
| Insert DNA (2000 bp) | 125 | 20 | 6.25 |
| Nuclease-free Water | | | 9.75 |
| T4 Ligase | | | 1 |
| Total | | | 20 |

All components were pipetted together on ice, starting with the mixing of DNA and water, addition of buffer and finally addition of the enzyme. The reaction was gently mixed by pipetting up and down, briefly microfuged and then incubated at room temperature for 10 minutes. After incubation, the T4 ligase was heat inactivated at 65° C. for 10 minutes. The sample was chilled on ice. In a final step, 10-beta competent E. coli cells were transformed with 2 μl of the ligated plasmid (see below).

### Cloning Via R-Site Assembly:

For assembly, all DNA fragments with the R-sites at each end were pipetted together on ice. An equimolar ratio (0.05 ng) of all fragments was used, as recommended by the manufacturer, when more than 4 fragments are being assembled. One-half of the reaction mix was embodied by NEBuilder HiFi DNA Assembly Master Mix. The total reaction volume was 40 μl and was reached by a fill-up with PCR-clean water.

In the following Table, an exemplary pipetting scheme is depicted.

TABLE
Assembly Reaction Mix

| | | pmol | ng | conc. | |
|---|---|---|---|---|---|
| component | bp | (set point) | (set point) | [ng/μl] | μl |
| Insert 1 | 2800 | 0.05 | 88.9 | 21 | 4.23 |
| Insert 2 | 2900 | 0.05 | 90.5 | 35 | 2.59 |
| Insert 3 | 4200 | 0.05 | 131.6 | 35.5 | 3.71 |
| Insert 4 | 3600 | 0.05 | 110.7 | 23 | 4.81 |
| Vector | 4100 | 0.05 | 127.5 | 57.7 | 2.21 |
| NEBuilder HiFi DNA | | | | | 20 |
| Assembly Master Mix | | | | | |
| PCR-clean Water | | | | | 2.45 |
| Total | | | | | 40 |

After set up of the reaction mixture, the tube was incubated in a thermocycler at constantly 50° C. for 60 minutes. After successful assembly, 10-beta competent E. coli bacteria were transformed with 2 μl of the assembled plasmid DNA (see below).

Transformation 10-beta competent E. coli cells:

For transformation, the 10-beta competent E. coli cells were thawed on ice. After that, 2 μl of plasmid DNA were pipetted directly into the cell suspension. The tube was flicked and put on ice for 30 minutes. Thereafter, the cells were placed into the 42° C.-warm thermal block and heat-shocked for exactly 30 seconds. Directly afterwards, the cells were chilled on ice for 2 minutes. 950 μl of NEB 10-beta outgrowth medium were added to the cell suspension. The cells were incubated under shaking at 37° C. for one hour. Then, 50-100 μl were pipetted onto a pre-warmed (37° C.) LB-Amp agar plate and spread with a disposable spatula. The plate was incubated overnight at 37° C. Only bacteria, which have successfully incorporated the plasmid, carrying the resistance gene against ampicillin, can grow on these plates. Single colonies were picked the next day and cultured in LB-Amp medium for subsequent plasmid preparation.

### Bacterial Culture:

Cultivation of E. coli was done in LB-medium, short for Luria Bertani, which was spiked with 1 ml/L 100 mg/ml ampicillin resulting in an ampicillin concentration of 0.1 mg/ml. For the different plasmid preparation quantities, the following amounts were inoculated with a single bacterial colony.

TABLE
E. coli cultivation volumes

| | volume LB-Amp | incubation time |
|---|---|---|
| quantity plasmid preparation | medium [ml] | [h] |
| Mini-Prep 96-well (EpMotion) | 1.5 | 23 |
| Mini-Prep 15 ml-tube | 3.6 | 23 |
| Maxi-Prep | 200 | 16 |

For Mini-Prep, a 96-well 2 ml deep-well plate was filled with 1.5 ml LB-Amp medium per well. The colonies were picked and the toothpick was tuck in the medium. When all colonies were picked, the plate closed with a sticky air porous membrane. The plate was incubated in a 37° C. incubator at a shaking rate of 200 rpm for 23 hours.

For Mini-Preps a 15 ml-tube (with a ventilated lid) was filled with 3.6 ml LB-Amp medium and equally inoculated with a bacterial colony. The toothpick was not removed but left in the tube during incubation. Like the 96-well plate, the tubes were incubated at 37° C., 200 rpm for 23 hours.

For Maxi-Prep 200 ml of LB-Amp medium were filled into an autoclaved glass 1 L Erlenmeyer flask and inoculated with 1 ml of bacterial day-culture, which was roundabout 5 hours old. The Erlenmeyer flask was closed with a paper plug and incubated at 37° C., 200 rpm for 16 hours.

### Plasmid Preparation:

For Mini-Prep, 50 μl of bacterial suspension were transferred into a 1 ml deep-well plate. After that, the bacterial cells were centrifuged down in the plate at 3000 rpm, 4° C. for 5 min. The supernatant was removed and the plate with the bacteria pellets placed into an EpMotion. After ca. 90 minutes, the run was done and the eluted plasmid-DNA could be removed from the EpMotion for further use.

For Mini-Prep, the 15 ml tubes were taken out of the incubator and the 3.6 ml bacterial culture splitted into two 2 ml Eppendorf tubes. The tubes were centrifuged at 6,800×g in a tabletop microcentrifuge for 3 minutes at room temperature. After that, Mini-Prep was performed with the Qiagen QIAprep Spin Miniprep Kit according to the manufacturer's instructions. The plasmid DNA concentration was measured with Nanodrop.

Maxi-Prep was performed using the Macherey-Nagel NucleoBond® Xtra Maxi EF Kit according to the manufacturer's instructions. The DNA concentration was measured with Nanodrop.

### Ethanol Precipitation:

The volume of the DNA solution was mixed with the 2.5-fold volume ethanol 100%. The mixture was incubated at −20° C. for 10 min. Then the DNA was centrifuged for 30 min. at 14,000 rpm, 4° C. The supernatant was carefully removed and the pellet washed with 70% ethanol. Again, the tube was centrifuged for 5 min. at 14,000 rpm, 4° C. The supernatant was carefully removed by pipetting and the pellet dried. When the ethanol was evaporated, an appropriate amount of endotoxin-free water was added. The DNA was given time to re-dissolve in the water overnight at 4° C. A small aliquot was taken and the DNA concentration was measured with a Nanodrop device.

### Example 2

### Plasmid Generation

### Expression Cassette Composition

For the expression of an antibody chain, a transcription unit comprising the following functional elements was used:
the immediate early enhancer and promoter from the human cytomegalovirus including intron A,
a human heavy chain immunoglobulin 5′-untranslated region (5′UTR),
a murine immunoglobulin heavy chain signal sequence,
a nucleic acid encoding the respective antibody chain,
the bovine growth hormone polyadenylation sequence (BGH pA), and
optionally the human gastrin terminator (hGT).

Beside the expression unit/cassette including the desired gene to be expressed, the basic/standard mammalian expression plasmid contains
an origin of replication from the vector pUC18 which allows replication of this plasmid in E. coli, and
a beta-lactamase gene which confers ampicillin resistance in E. coli.

### Front- and Back-Vector Cloning

To construct two-plasmid antibody constructs, antibody HC and LC fragments were cloned into a front vector backbone containing L3 and LoxFas sequences, and a back vector containing LoxFas and 2L sequences and a Pac selectable marker. The Cre recombinase plasmid pOG231 (Wong, E. T., et al., Nucl. Acids Res. 33 (2005) e147; O'Gorman, S., et al., Proc. Natl. Acad. Sci. USA 94 (1997) 14602-14607) was used for all RMCE processes.

The cDNAs encoding the respective antibody chains were generated by gene synthesis (Geneart, Life Technologies Inc.). The gene synthesis and the backbone-vectors were digested with HindIII-HF and EcoRI-HF (NEB) at 37° C. for 1 h and separated by agarose gel electrophoresis. The DNA-fragment of the insert and backbone were cut out from the agarose gel and extracted by QIAquick Gel Extraction Kit (Qiagen). The purified insert and backbone fragment was ligated via the Rapid Ligation Kit (Roche) following the manufacturer's protocol with an Insert/Backbone ratio of 3:1. The ligation approach was then transformed in competent E. coli DH5α via heat shock for 30 sec. at 42° C. and incubated for 1 h at 37° C. before they were plated out on agar plates with ampicillin for selection. Plates were incubated at 37° C. overnight.

On the following day clones were picked and incubated overnight at 37° C. under shaking for the Mini or Maxi-Preparation, which was performed with the EpMotion® 5075 (Eppendorf) or with the QIAprep Spin Mini-Prep Kit (Qiagen)/NucleoBond Xtra Maxi EF Kit (Macherey & Nagel), respectively. All constructs were sequenced to ensure the absence of any undesirable mutations (SequiServe GmbH).

In the second cloning step, the previously cloned vectors were digested with KpnI-HF/SalI-HF and SalI-HF/MfeI-HF with the same conditions as for the first cloning. The TI backbone vector was digested with KpnI-HF and MfeI-HF. Separation and extraction was performed as described above. Ligation of the purified insert and backbone was performed using T4 DNA Ligase (NEB) following the manufacturing protocol with an Insert/Insert/Backbone ratio of 1:1:1 overnight at 4° C. and inactivated at 65° C. for 10 min. The following cloning steps were performed as described above.

The cloned plasmids were used for the TI transfection and pool generation.

### Example 3

### Cultivation, Transfection, Selection and Single Cell Cloning

TI host cells were propagated in disposable 125 ml vented shake flasks under standard humidified conditions (95% rH, 37° C., and 5% CO₂) at a constant agitation rate of 150 rpm in a proprietary DMEM/F12-based medium. Every 3-4 days the cells were seeded in chemically defined medium containing selection marker 1 and selection marker 2 in effective concentrations with a concentration of 3×10E5 cells/ml. Density and viability of the cultures were measured with a Cedex HiRes cell counter (F. Hoffmann-La Roche Ltd, Basel, Switzerland).

For stable transfection, equimolar amounts of front and back vector were mixed. 1 μg Cre expression plasmid was added per 5 μg of the mixture, i.e. 5 μg Cre expression plasmid or Cre mRNA was added to 25 μg of the front- and back-vector mixture.

Two days prior to transfection TI host cells were seeded in fresh medium with a density of 4×10E5 cells/ml. Transfection was performed with the Nucleofector device using the Nucleofector Kit V (Lonza, Switzerland), according to the manufacturer's protocol. 3×10E7 cells were transfected with a total of 30 μg nucleic acids, i.e. either with 30 μg plasmid (5 μg Cre plasmid and 25 μg front- and back-vector mixture) or with 5 μg Cre mRNA and 25 μg front- and back-vector mixture. After transfection, the cells were seeded in 30 ml medium without selection agents.

On day 5 after seeding the cells were centrifuged and transferred to 80 mL chemically defined medium containing puromycin (selection agent 1) and 1-(2′-deoxy-2′-fluoro-1-beta-D-arabinofuranosyl-5-iodo)uracil (FIAU; selection agent 2) at effective concentrations at 6×10E5 cells/ml for selection of recombinant cells. The cells were incubated at 37° C., 150 rpm. 5% CO₂, and 85% humidity from this day on without splitting. Cell density and viability of the culture was monitored regularly. When the viability of the culture started to increase again, the concentrations of selection agents 1 and 2 were reduced to about half the amount used before. In more detail, to promote the recovering of the cells, the selection pressure was reduced if the viability is >40% and the viable cell density (VCD) is >0.5×10E6 cells/mL. Therefore, 4×10E5 cells/ml were centrifuged and resuspended in 40 ml selection media II (chemically-defined medium, ½ selection marker 1 & 2). The cells were incubated with the same conditions as before and also not splitted.

Ten days after starting selection, the success of Cre mediated cassette exchange was checked by flow cytometry measuring the expression of intracellular GFP and extracellular heterologous polypeptide bound to the cell surface. An APC antibody (allophycocyanin-labeled F(ab′)2 Fragment goat anti-human IgG) against human antibody light and heavy chain was used for FACS staining. Flow cytometry was performed with a BD FACS Canto II flow cytometer (BD, Heidelberg, Germany). Ten thousand events per sample were measured. Living cells were gated in a plot of forward scatter (FSC) against side scatter (SSC). The live cell gate was defined with non-transfected TI host cells and applied to all samples by employing the FlowJo 7.6.5 EN software (TreeStar, Olten, Switzerland). Fluorescence of GFP was quantified in the FITC channel (excitation at 488 nm, detection at 530 nm). Heterologous polypeptide was measured in the APC channel (excitation at 645 nm, detection at 660 nm). Parental CHO cells, i.e. those cells used for the generation of the TI host cell, were used as a negative control with regard to GFP and heterologous polypeptide expression. Fourteen to twenty-one days after the selection had been started, the viability exceeded 90% and selection was considered as complete.

After selection, the pool of stably transfected cells can be subjected to single-cell cloning by limiting dilution. For this purpose, cells are stained with Cell Tracker Green (Thermo Fisher Scientific, Waltham, Mass.) and plated in 384-well plates with 0.6 cells/well. For single-cell cloning and all further cultivation steps, selection agent 2 is omitted from the medium. Wells containing only one cell are identified by bright field and fluorescence based plate imaging. Only wells that contain one cell are further considered. Approximately three weeks after plating colonies are picked from confluent wells and further cultivated in 96-well plates.

### Example 4

### FACS Screening

FACS analysis was performed to check the transfection efficiency and the RMCE efficiency of the transfection. 4×10E5 cells of the transfected approaches were centrifuged (1200 rpm, 4 min.) and washed twice with 1 mL PBS. After the washing steps with PBS the pellet was resuspended in 400 μL PBS and transferred in FACS tubes (Falcon® Round-Bottom Tubes with cell strainer cap; Corning). The measurement was performed with a FACS Canto II and the data were analyzed by the software FlowJo.

### Example 5

### Fed-Batch Cultivation

Fed-batch production cultures were performed in shake flasks or Ambr 15 vessels (Sartorius Stedim) with proprietary chemically defined medium. Cells were seeded at 2×10E6 cells/ml on day 0. Cultures received proprietary feed medium on days 3, 7, and 10. Viable cell count (VCC) and percent viability of cells in culture was measured on days 0, 3, 7, 10, and 14 using a Cedex HiRes instrument (Roche Diagnostics GmbH, Mannheim, Germany). Glucose, lactate and product titer concentrations were measured on days 3, 5, 7, 10, 12 and 14 using a Cobas Analyzer (Roche Diagnostics GmbH, Mannheim, Germany). The supernatant was harvested 14 days after start of fed-batch cultivation by centrifugation (10 min, 1000 rpm and 10 min, 4000 rpm) and cleared by filtration (0.22 μm). Day 14 titers were determined using protein A affinity chromatography with UV detection. Product quality was determined by Caliper's LabChip (Caliper Life Sciences).

### Example 6

### RNP-Based CRISPR-Cas9 Gene Knockouts in CHO Cells

### Material/Resources:

Geneious 11.1.5 software for guide and primer design
CHO TI host cell line; cultivation state: day 30-60
TrueCut™ Cas9 Protein v2 (Invitrogen™)
TrueGuide Synthetic gRNA (custom designed against target gene, 3 nm unmodified gRNA, Thermo Fisher)
TrueGuide™ sgRNA Negative Control, non-targeting 1 (Thermo Fisher)
medium (200 μg/ml Hygromycin B, 4 μg/ml selection agent 2)
DPBS—Dulbecco's Phosphate-Buffered Saline w/o Ca and Mg (Thermo Fisher)
Microplate 24 deep well plate (Agilent Technologies, Porvoir science) with cover (self-made)
Thin, long RNase, DNase, pyrogen free filter tips for loading OC-100 cassettes. (Biozyme)
Hera Safe Hood (Thermo Fisher)
Cedex HiRes Analyzer (Innovatis)
Liconic Incubator Storex IC
HyClone electroporation buffer
MaxCyte OC-100 cassettes
MaxCyte STX electroporation system

### CRISPR-Cas9 RNP Delivery

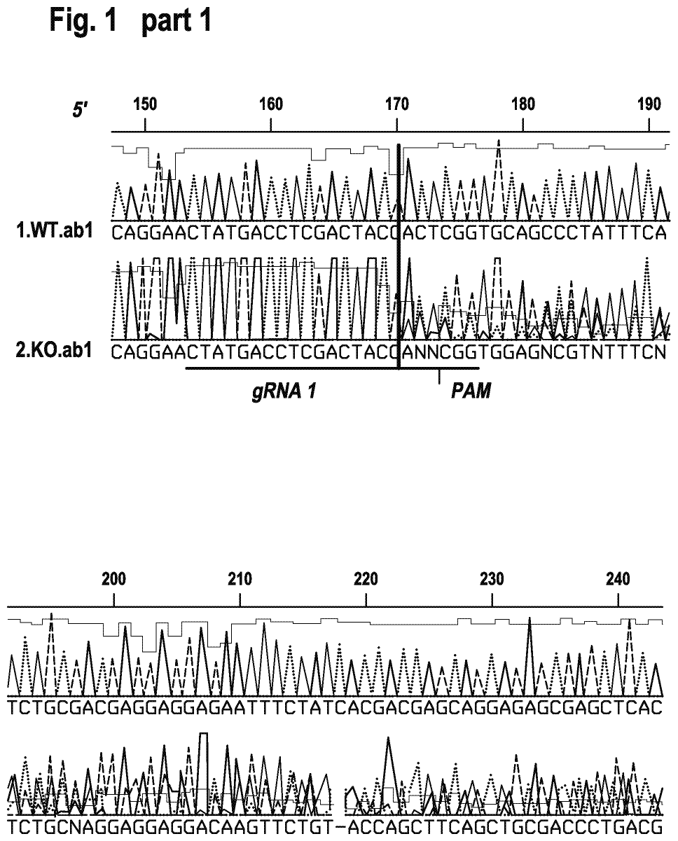

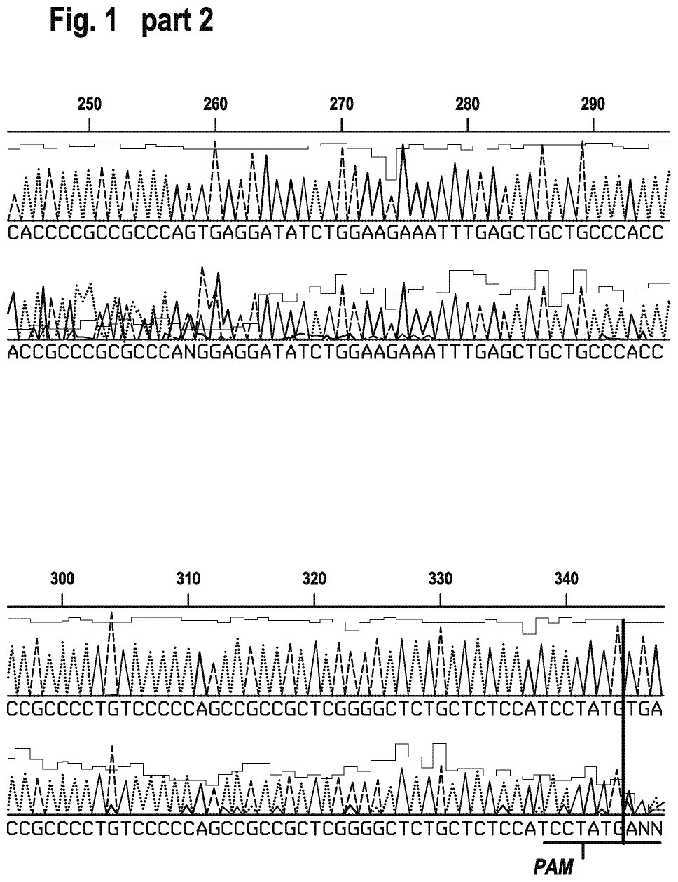

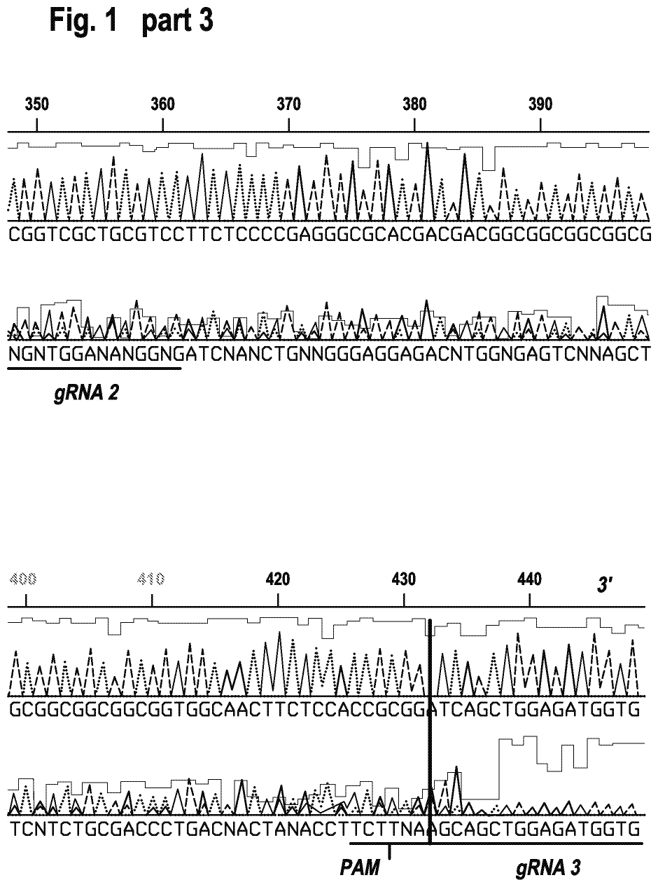

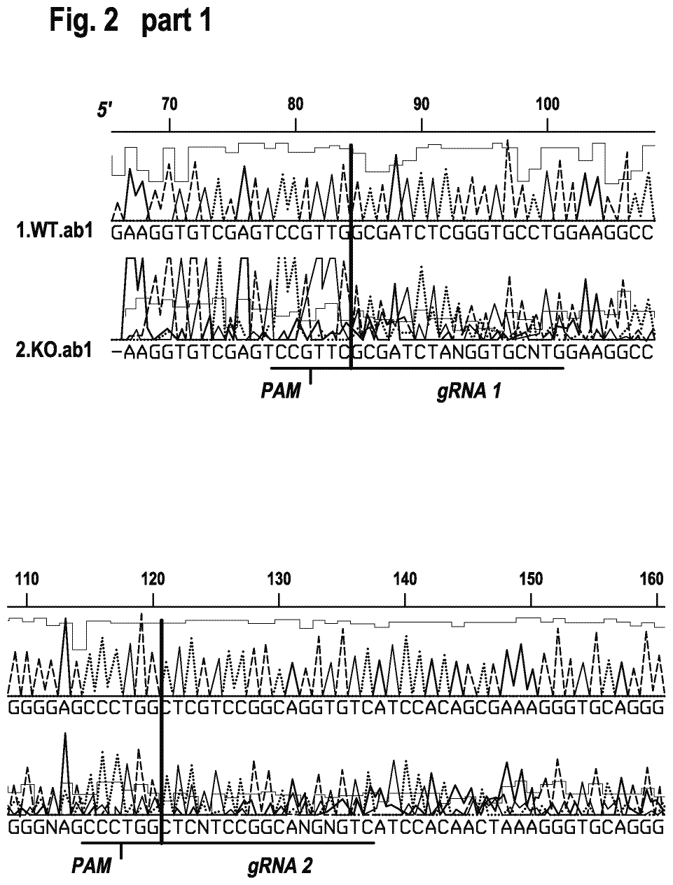

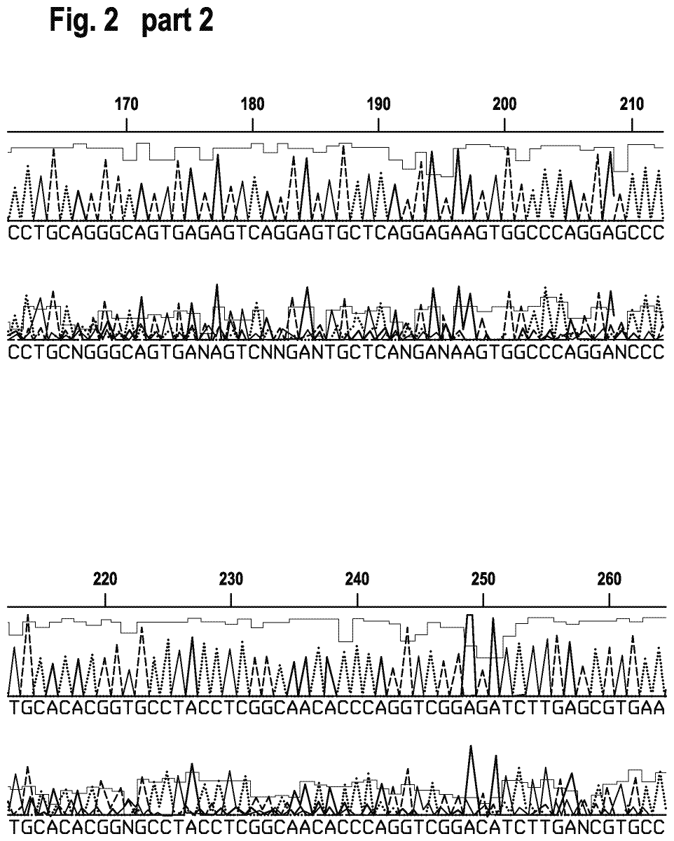

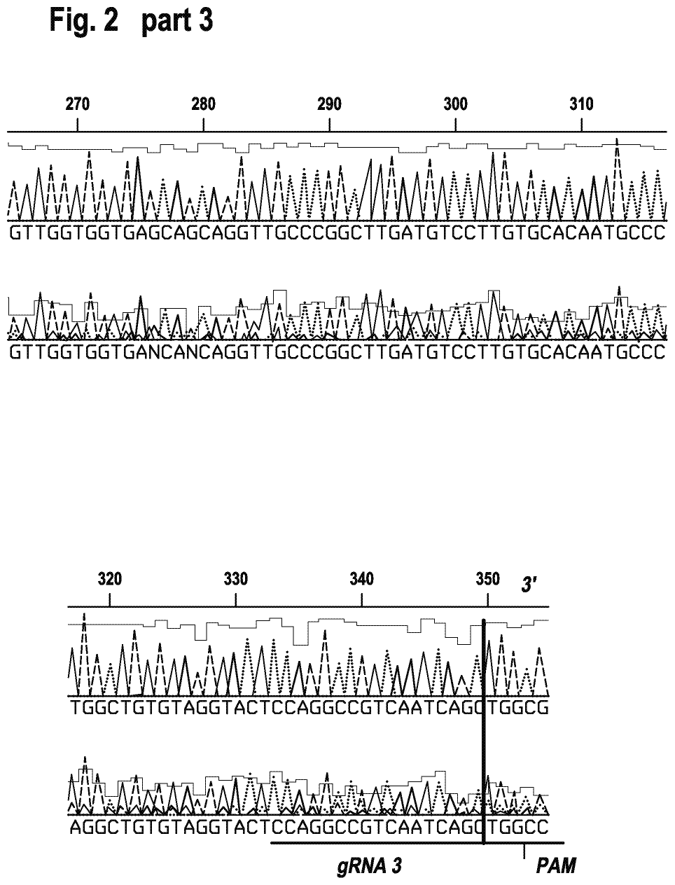

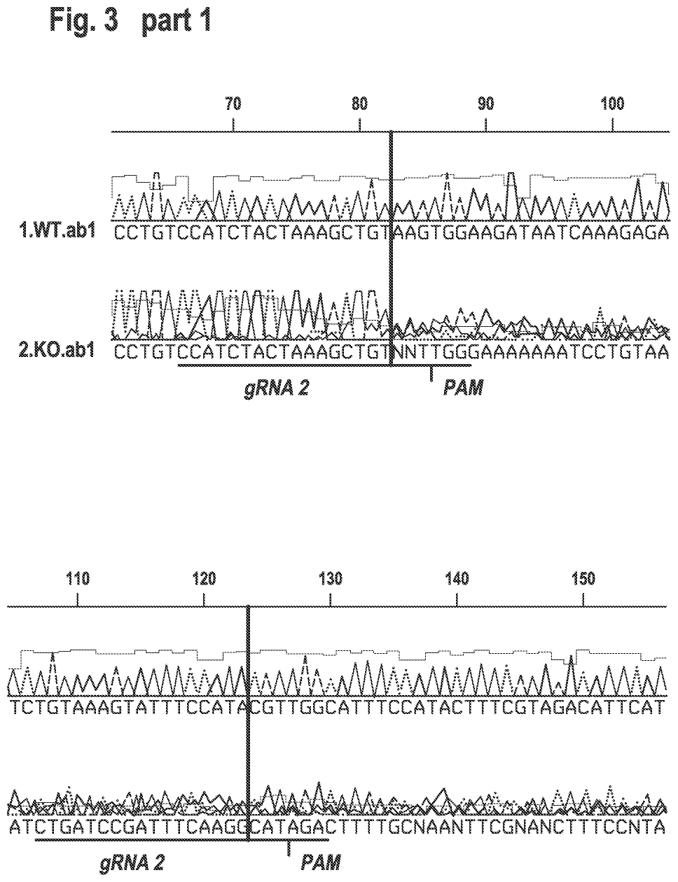

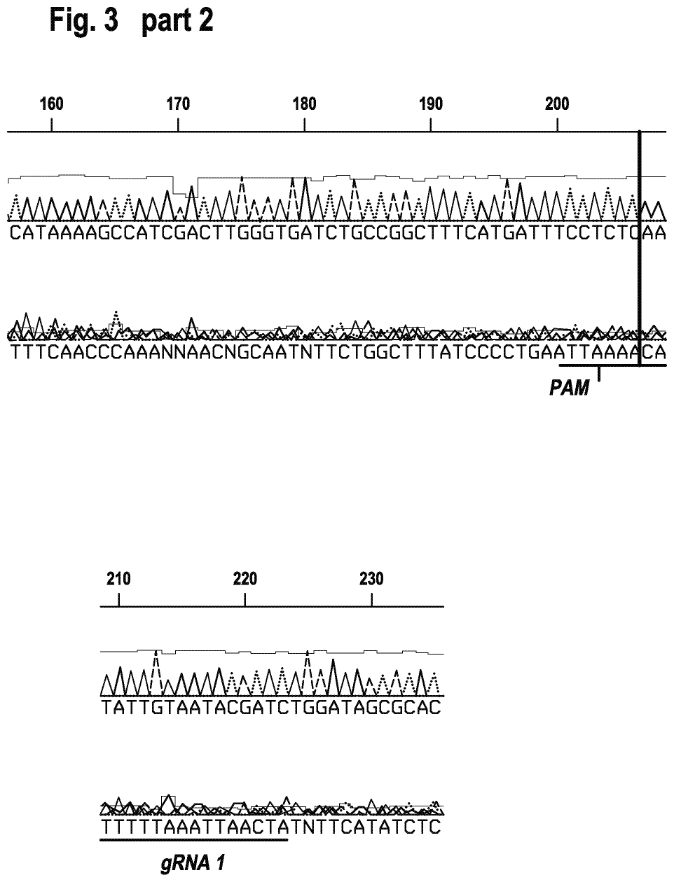

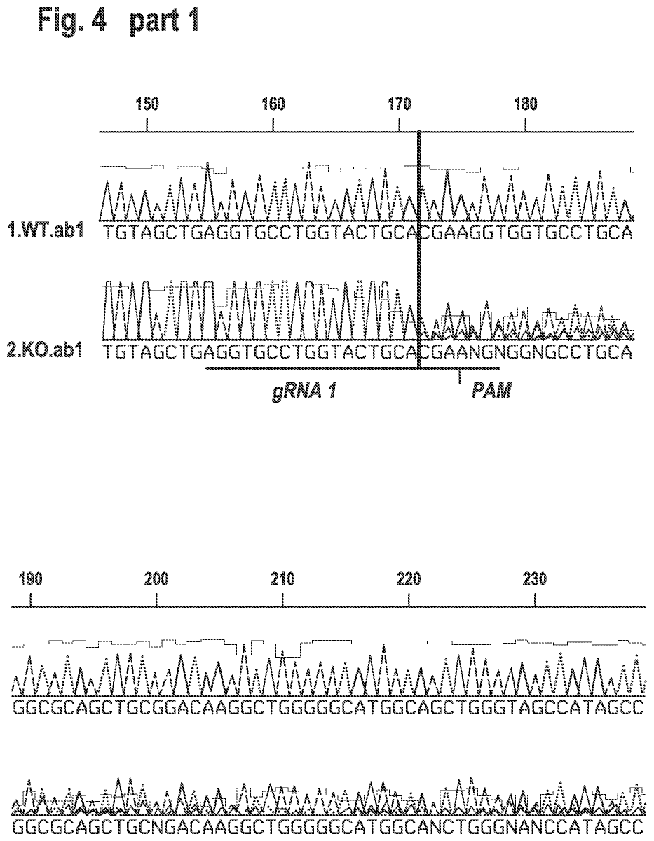

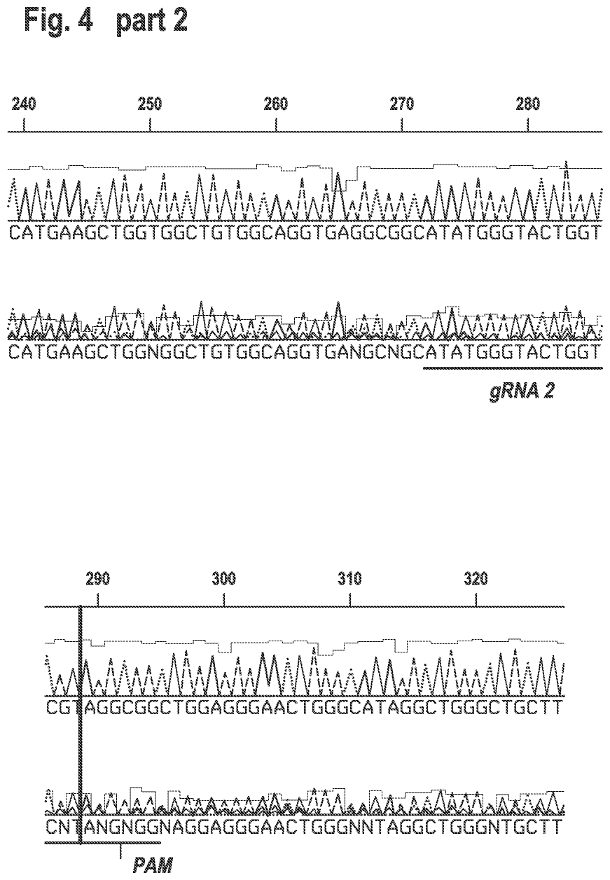

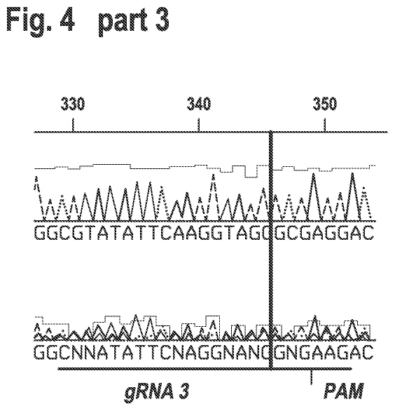

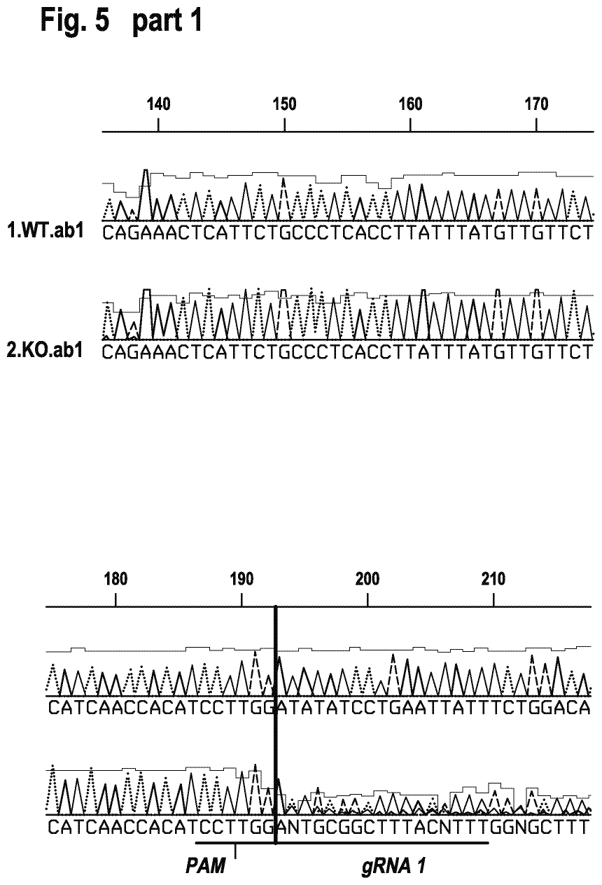

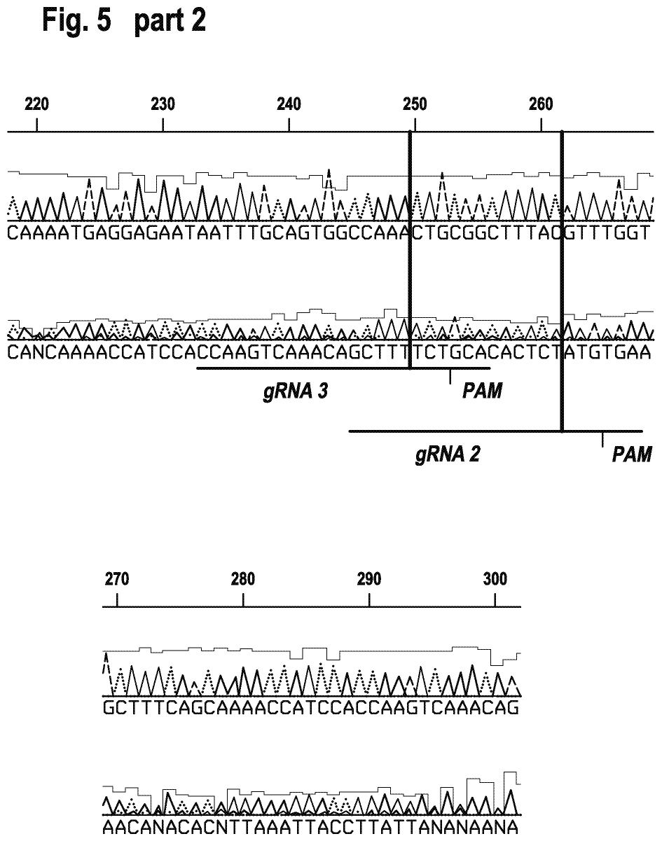

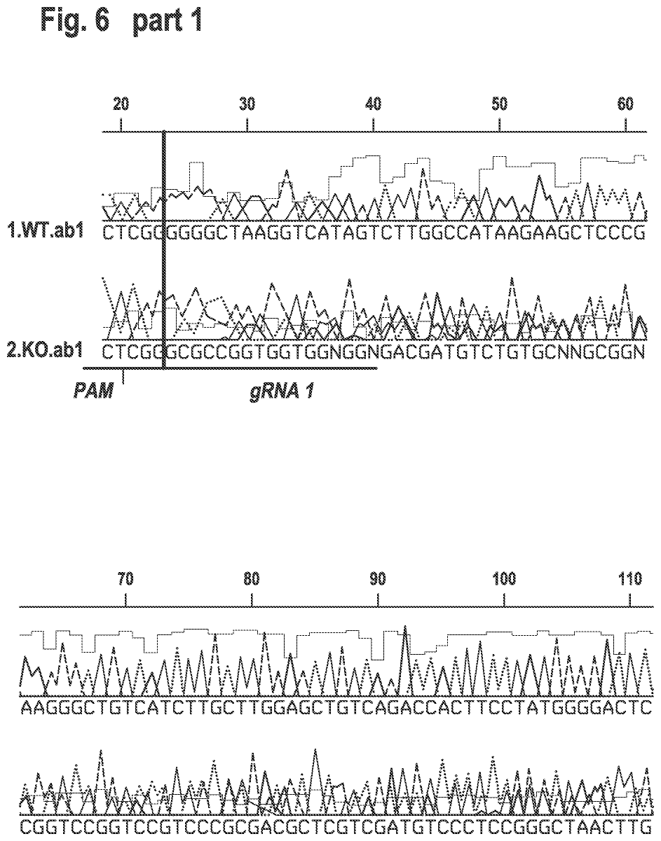

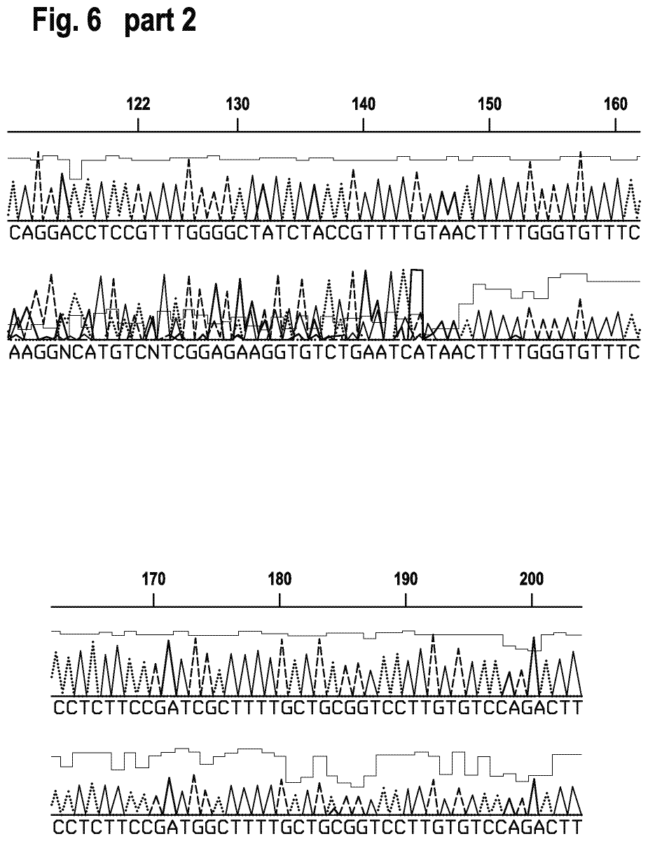

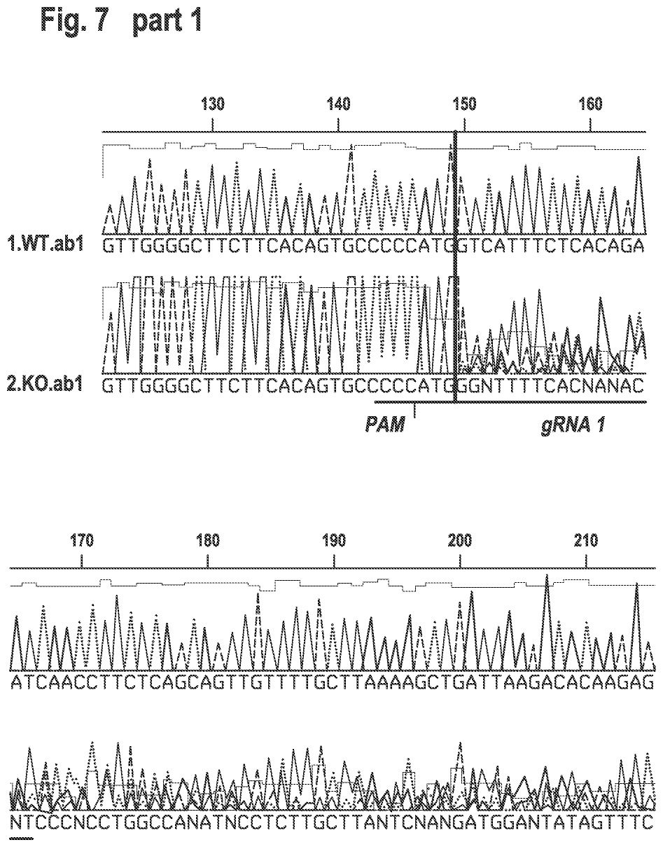

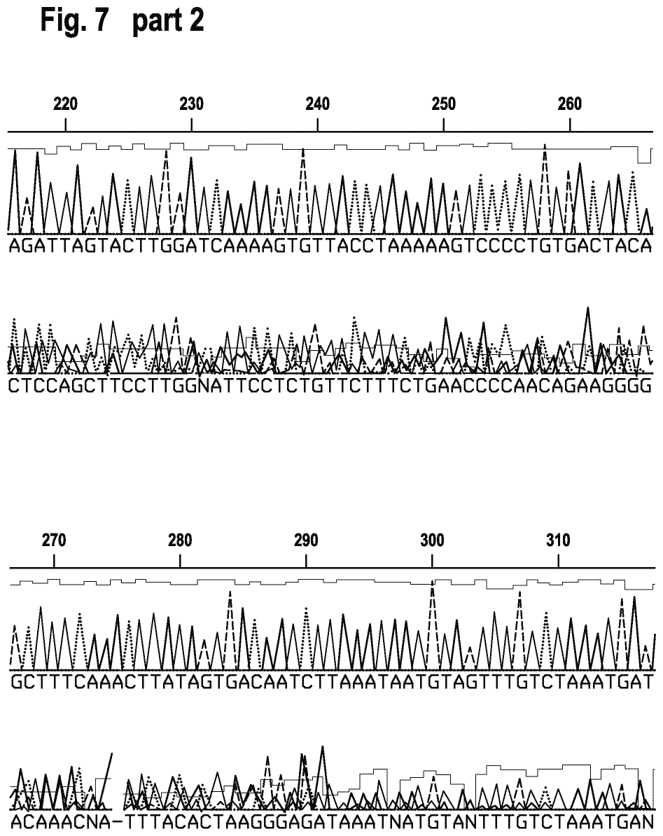

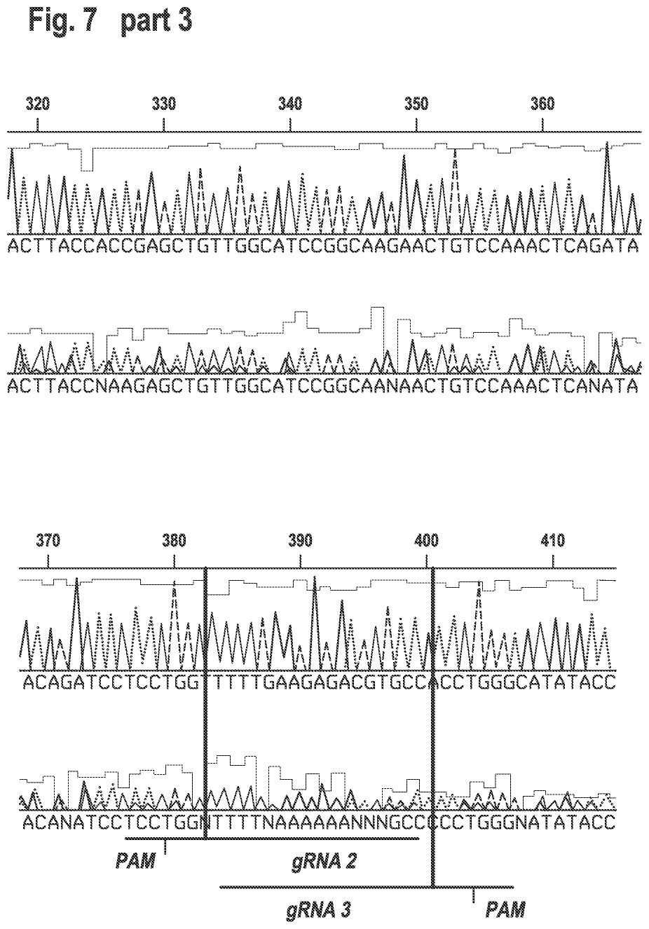

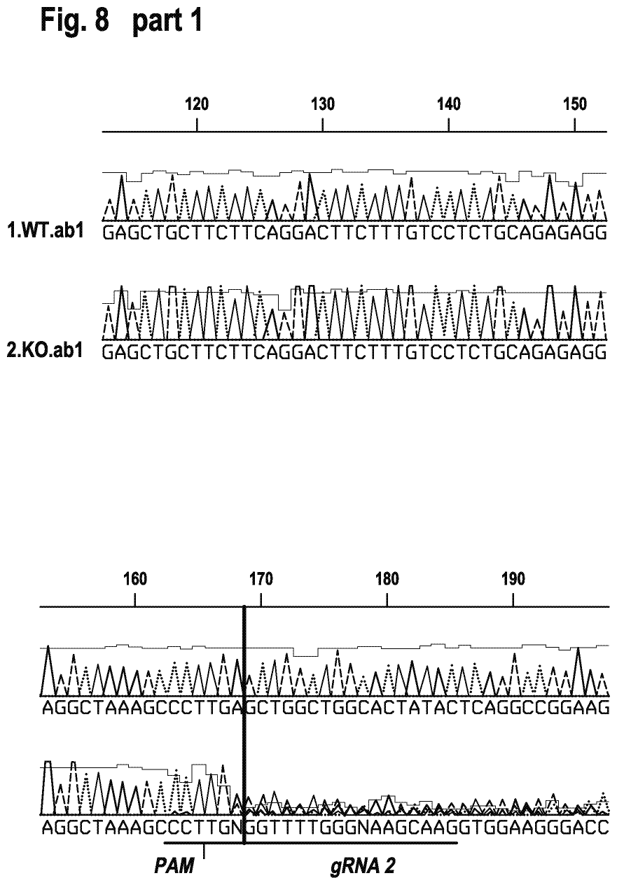

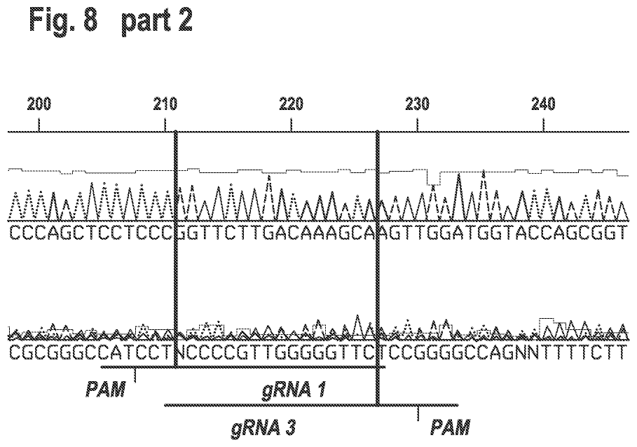

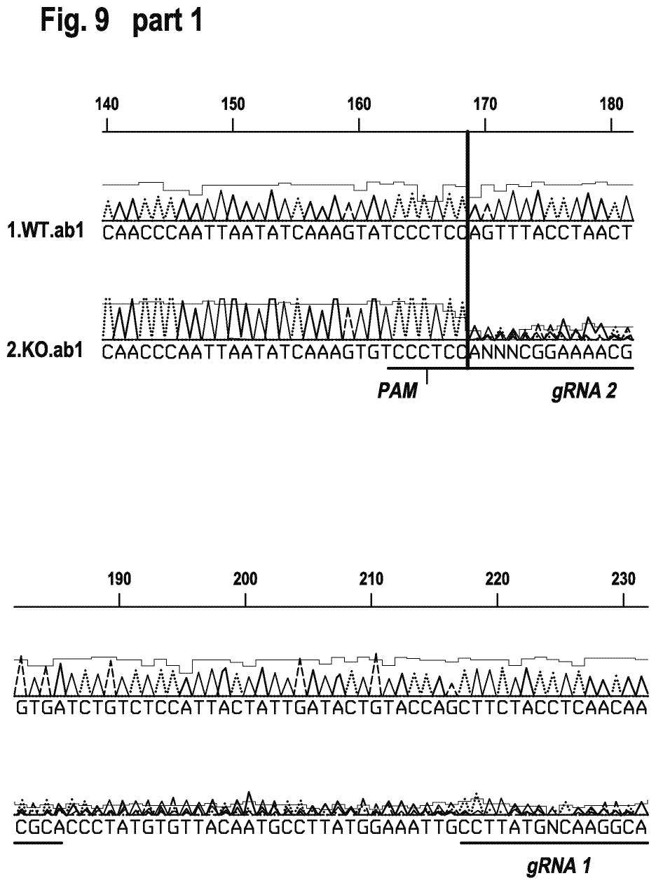

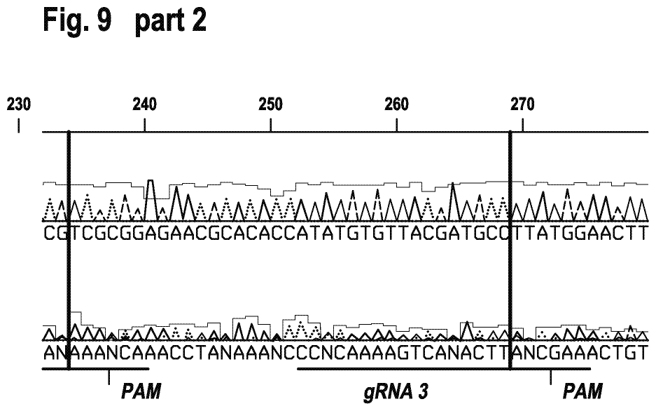

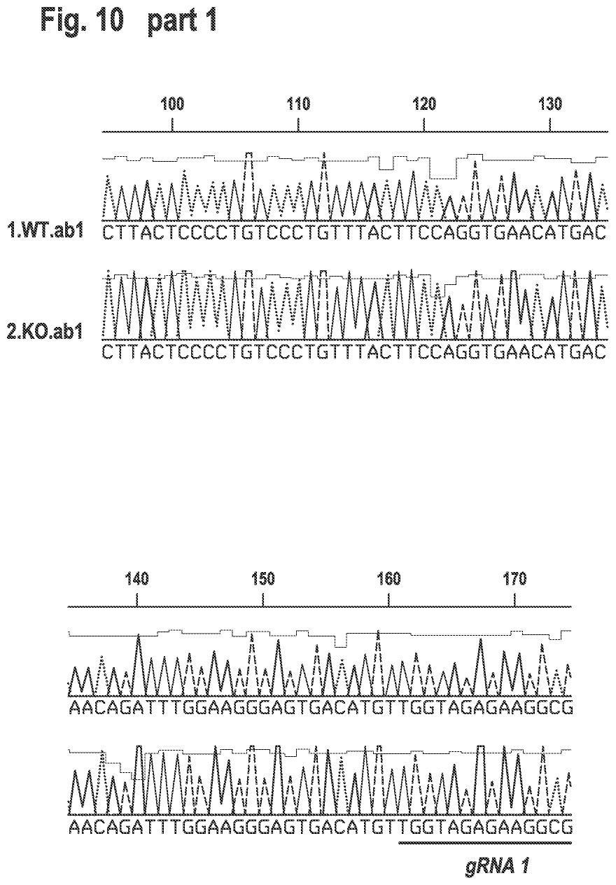

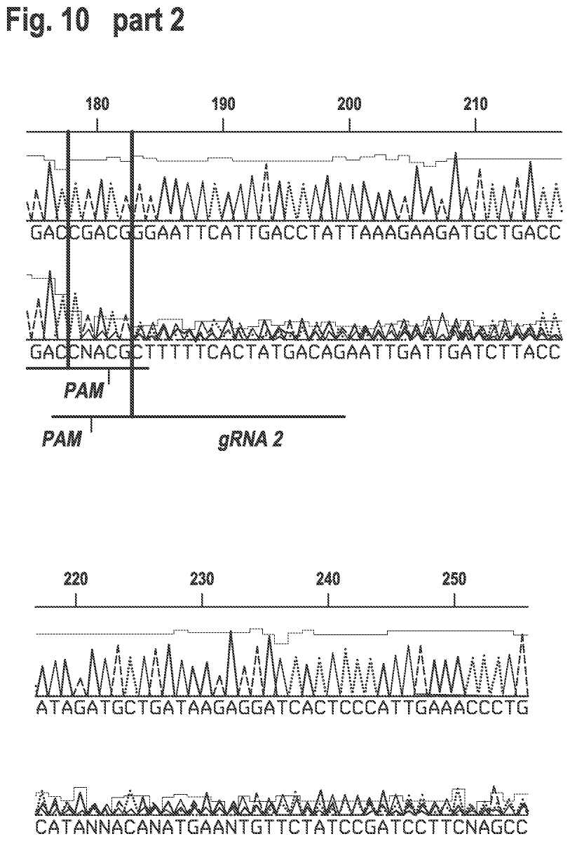

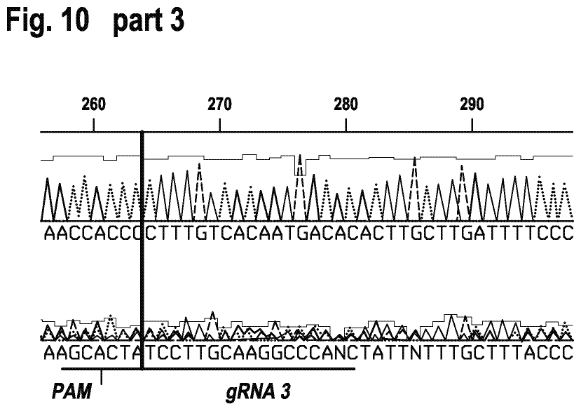

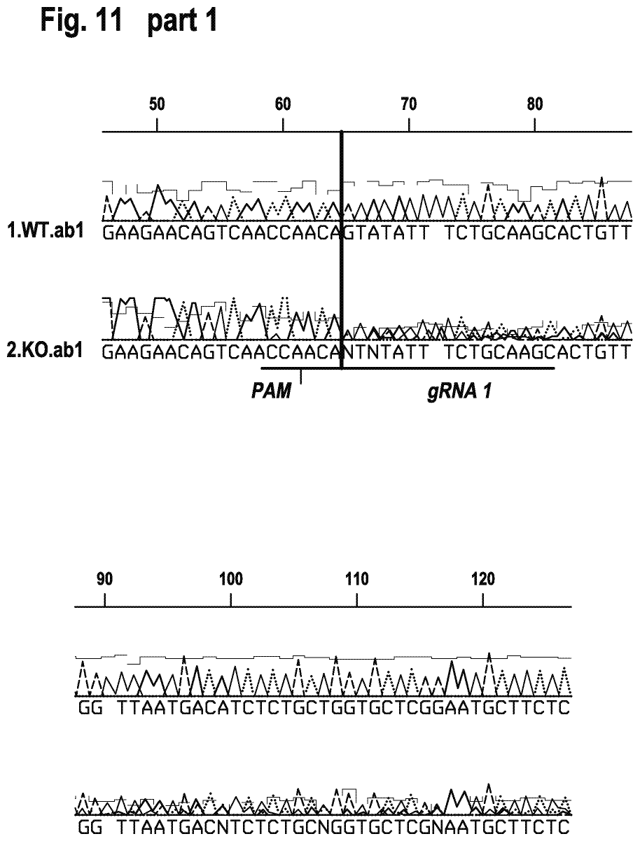

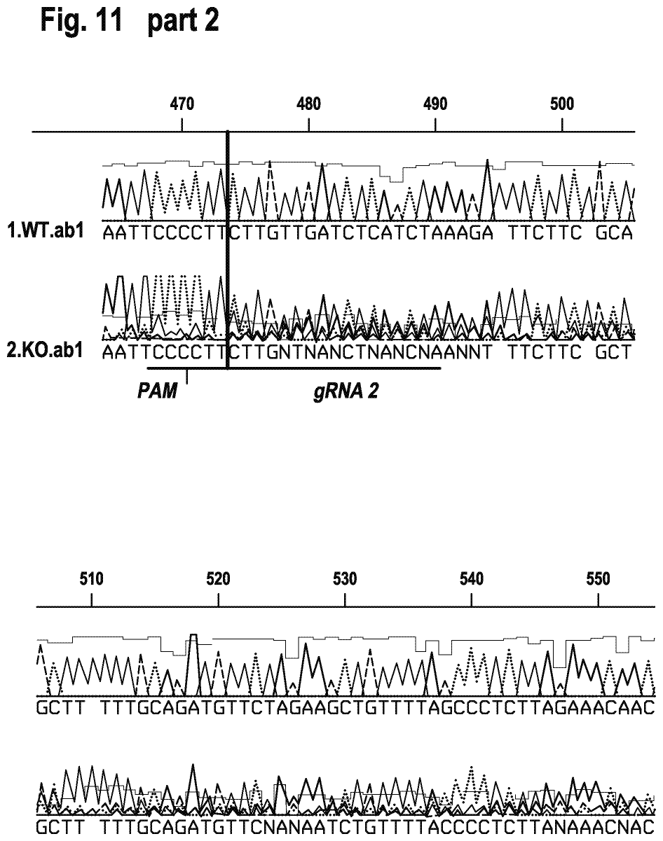

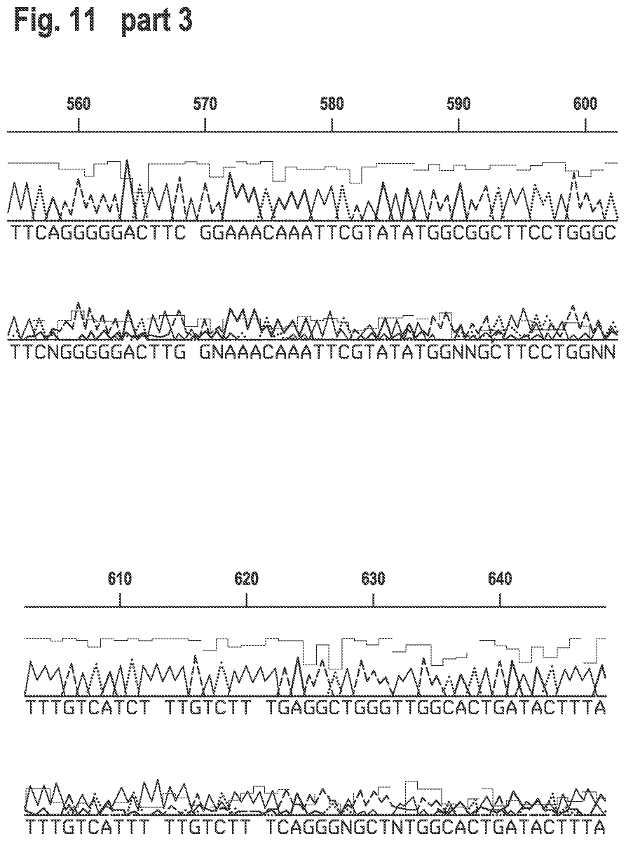

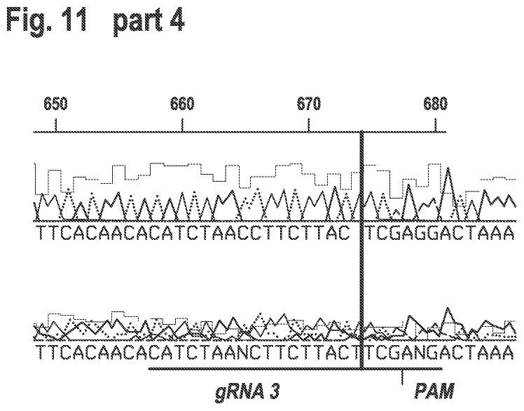

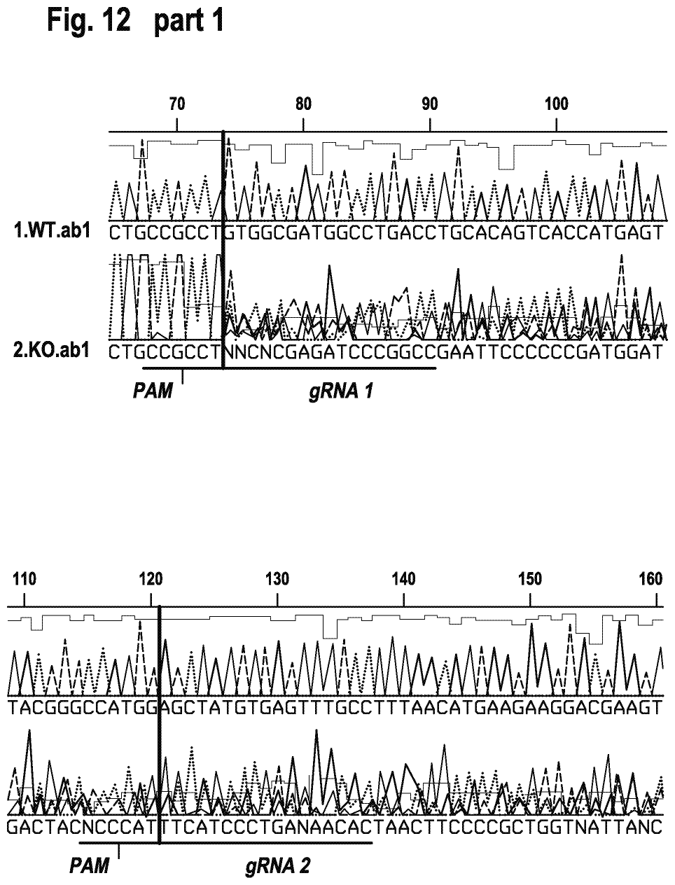

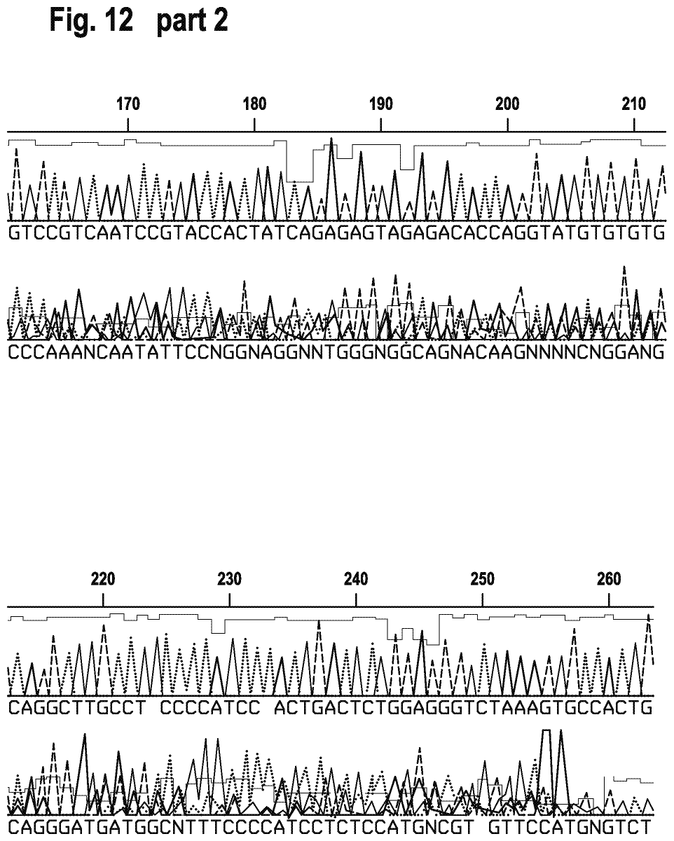

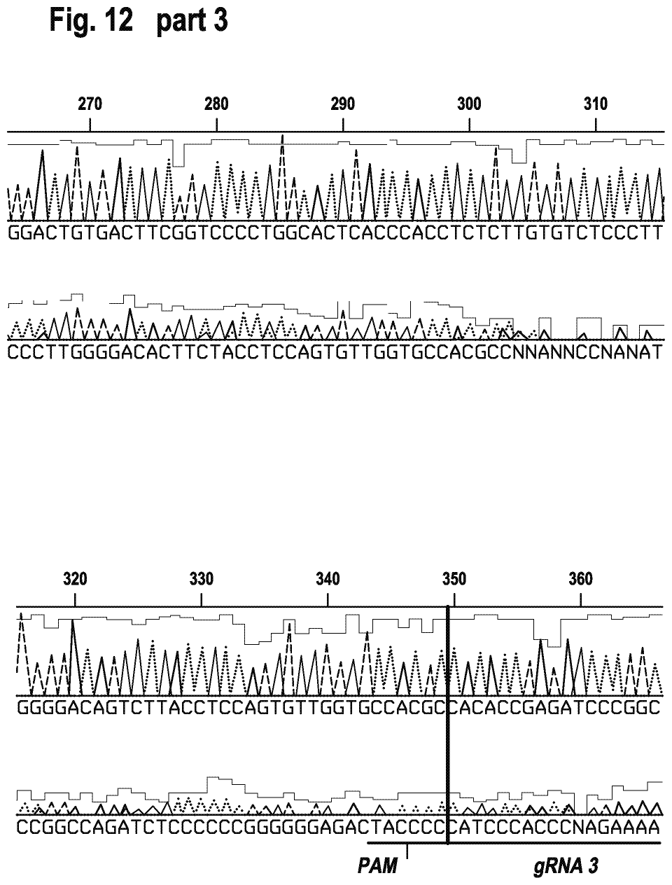

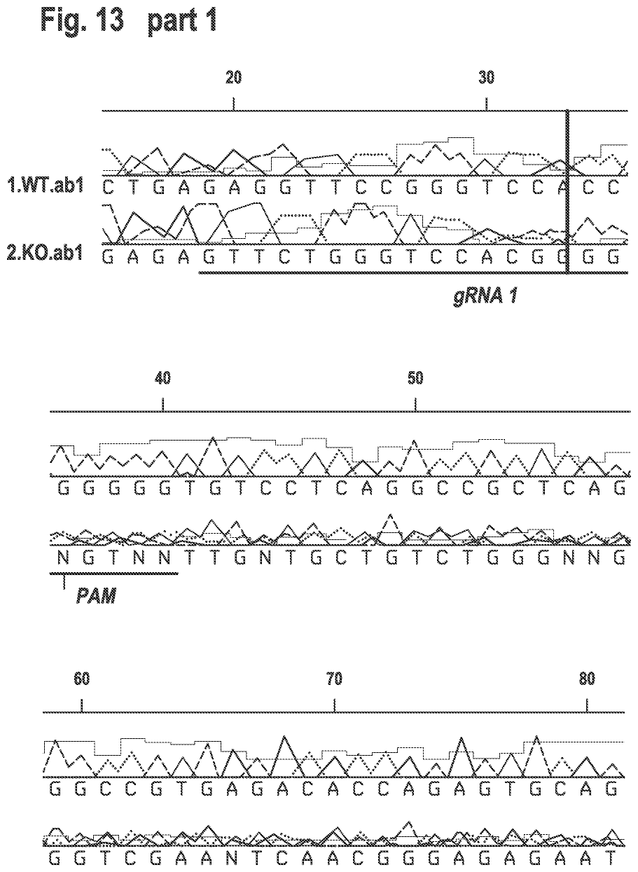

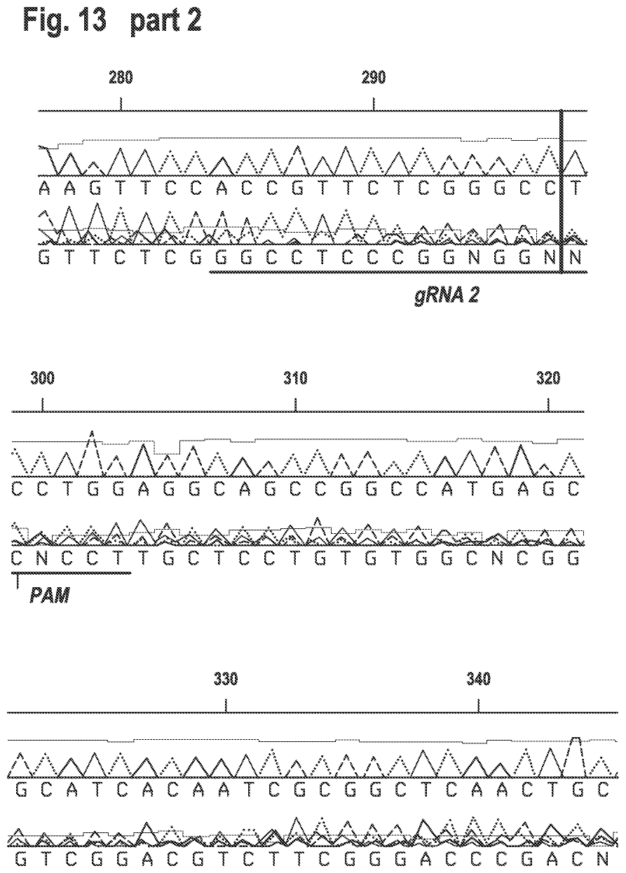

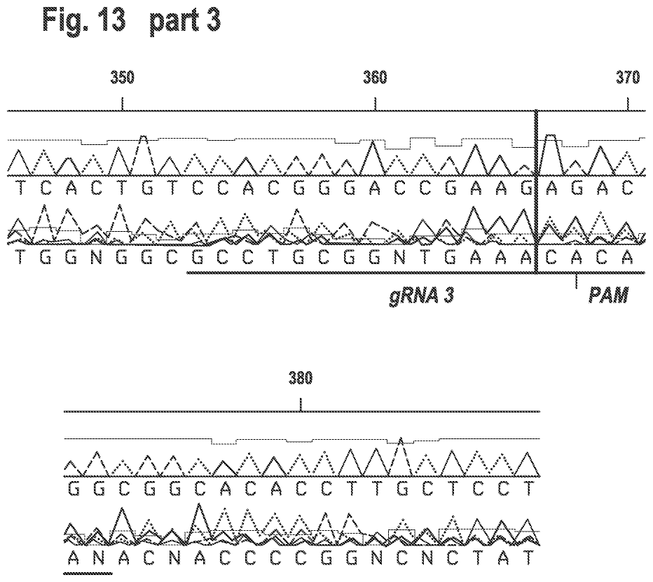

RNPs were preassembled by mixing 5 μg Cas9 with 1 μg gRNA mix (equal ratio of each gRNA—see Table below for exemplary genes-specific gRNA sequences) in 10 μL PBS and incubated for 20 minutes at RT. Cells with a concentration between 2-4×10E6 cell/mL were centrifuged (3 minutes, 300 g) and washed with 500 μL PBS. After the washing step, the cells were again centrifuged (3 minutes at 300 g) and resuspended in 90 μL HyClone electroporation buffer. The pre-incubated RNP mix was added to the cells and incubated for 5 minutes. The cell/RNP solution was then transferred into an OC-100 cuvette and electroporated with program “CHO2” using a MaxCyte electroporation system. Immediately after electroporation, the cell suspension was transferred into a 24 dwell and incubated at 37° C. for 30 minutes. Fresh and pre-warmed medium was added to result in a final cell concentration of 1×10E6 and incubated at 37° C. with shaking at 350 rpm for cell expansion. For genomic DNA preparation (day 6 or 8), QuickExtract kit (Lucigen) was added to the cells and served as a PCR template. Specific gene amplicons were PCR-amplified using standard Q5 Hot Start Polymerase protocol (NEB) and gene-specific primers that span the gRNA target sites (see Table below for examples). The respective amplicon was purified using QIAquick PCR purification kit (Qiagen) and analyzed by Sanger sequencing by Eurofins Genomics GmbH to verify gene inactivation by knockout (see FIGS. 1 to 13 for examples).

| gene | verification primer | | gRNA | | |
|---|---|---|---|---|---|
| name | forward | reverse | 1 | 2 | 3 |
| (short) | (SEQ ID NO) | (SEQ ID NO) | (SEQ ID NO) | (SEQ ID NO) | (SEQ ID NO) |
| PARP-1 | CTCTCTGC | ATGTAAGT | TTGCTTTGT | TATAGTGC | CGGTTCTT |
| | AGTTCCCT | GCAAGGT | CAAGAACC | CAGCCAG | GACAAAG |
| | AC (76) | GTC (77) | GGG (39) | CTCAA (40) | CAAGT (41) |
| ATM | GTAAAGA | GAAGGTTT | CTTCTACCT | TCACAGTT | ATATGTGT |
| | GCTAGCCA | ACAGGCT | CAACAACG | AGGTAAA | TACGATGC |
| | GAAG (78) | GAG (79) | TCG (42) | CTGGA (43) | CTTA (44) |
| MYC | CACACAC | CTTGATGA | CTATGACCT | GGACGCA | CACCATCT |
| | ACACTTGG | AGGTCTCG | CGACTACG | GCGACCGT | CCAGCTGA |
| | AAG (60) | TC (61) | ACT (15) | CACAT (16) | TCCG (17) |
| RBM38 | TCTCATGT | GTTTTGTA | AGGTGCCT | ATATGGGT | CGTATATT |
| | CCTTCCTC | GATGGGG | GGTACTGC | ACTGGTCG | CAAGGTA |
| | AG (68) | TTG (69) | ACGA (27) | TAGG (28) | GGGCG (29) |
| BARD1 | GGCTAAG | CAACACAT | GCTTGCAG | TAGCTGAG | CATCTAAC |
| | GGAGTTAT | CTAGGAC | AAAATATA | ATCAACA | CTTCTTAC |
| | CTG (82) | AGG (83) | CTGT (48) | AGAAG (49) | TTCG (50) |
| CDKN1A | TACCTGTC | GGGAAGA | GAGAGGTT | ACCGTTCT | CCACGGG |
| | CCTACCTG | TTGTGACT | CCGGGTCC | CGGGCCTC | ACCGAAG |
| | TC (88) | TATG (89) | ACCG (57) | CTGG (58) | AGACGG |
| | | | | | (59) |
| CDK12 | CAGGACTC | GATTCAGA | ACTATGACC | TTAGCAAG | GCTTGTGC |
| | TTCTTGTA | CACCTTCT | TTAGCCCCC | TCTCGGGA | TTCGACAC |
| | GGAG (72) | CC (73) | CG (33) | CCGC (34) | CAAG (35) |
| NF1 | ACAGAGC | CTGTAAGA | AATAATTCA | AATTTGCA | CCAAACTG |
| | TAAGAGC | CCCTAATA | GGATATATC | GTGGCCA | CGGCTTTA |
| | CTTC (70) | GTATGAC | CA (30) | AACTG (31) | CGTT (32) |
| | | (71) | | | |
| SIN3A | GTGGCCTA | CTCCCTTA | ATTCTGTGA | ACGTCTCT | TTTTGAAG |
| | TACTAACG | GTGTGTAT | GAAATGAC | TCAAAAA | AGACGTG |
| | TG (74) | CG (75) | CAT (36) | CCAGG (37) | CCACC (38) |
| STK11 | CTAGAGA | TCTGGCCT | CAGCCACC | GACACCTG | CCAGGCC |
| | AAACCCA | TCTAATTG | CGAGATCG | CCGGACG | GTCAATCA |
| | CAGTTC | TC (63) | CCAA (18) | AGCCA (19) | GCTGG (20) |
| | (62) | | | | |
| SMAD3 | ACTTCACT | GAACAAC | GGTCAGGC | GGCAAAC | GCCGGGA |
| | GACACCTT | GACATGG | CATCGCCAC | TCACATAG | TCTCGGTG |
| | CTG (86) | AGAG (87) | AGG (54) | CTCCA (55) | TGGCG (56) |
| SMAD4 | TAGGTGTG | AGGTCTTC | CTGCCTGCC | TCTGCAAC | GTAACAAT |
| | TATGGTGC | TCCTAGTG | AGAATACT | AGTCCTTC | AGGGCAG |
| | AG (64) | CTC (65) | GGC (21) | ACTA (22) | CTTGA (23) |
| HIF1AN | GTTCAGTA | CTCATCTC | TGTGTACCC | CTTCAAAC | ACAGGAT |
| | ATGGAAC | TATGGTGT | TGCTCTGAA | CAAGGTCC | ATACAGC |
| | CAG (84) | GC (85) | GT (51) | AGCA (52) | ATCGAG |
| | | | | | (53) |
| PPP2CB | CTTGTAAA | CCCACAA | GAGCGTATT | TGTAAAGT | CCATCTAC |
| | TACAGATC | GATTACTC | ACAATATTG | ATTTCCAT | TAAAGCTG |
| | CTGAG (66) | TAGC (67) | AG (24) | ACGT (25) | TAAG (26) |
| HIPK2 | ACGTACGT | GGTAAACT | TGGTAGAG | ATAGGTCA | GTGTCATT |
| | ATGTGAAT | ACAGTCTT | AAGGCGGA | ATGAATTC | GTGACAA |
| | CC (80) | AGGC (81) | CCGA (45) | CCGT (46) | AGGGG (47) |

### Example 7

### 4-Day Batch Cultivation

Batch production cultures were performed in 6 well, deep well plates or 24 well, deep well plates or shake flasks with proprietary chemically defined medium. Cells were seeded at 5×10E6 cells/ml. Viable cell count (VCC) and percent viability of cells in culture was measured on days 0, 2, 4 using a Cedex HiRes (Roche Diagnostics GmbH, Mannheim, Germany) or Cellavista (Synentec GmbH, Elmshom, Germany). Glucose concentration, lactate concentration and product titer were measured on days 0, 2, 4 using a Cobas analyzer (Roche Diagnostics GmbH, Mannheim, Germany). The supernatant was harvested 4 days after start of batch by centrifugation (10 min., 1000 rpm followed by 10 min., 4000 rpm) and cleared by filtration (0.22 μm). Titers were determined using protein A affinity chromatography with UV detection. Product quality was determined by Caliper's LabChip (Caliper Life Sciences).

### Example 8

### Fed-Batch Cultivation

Fed-batch production cultures were performed in shake flasks or Ambr 15 vessels (Sartorius Stedim) with proprietary chemically defined medium. Cells were seeded at 2×10E6 cells/ml. Cultures received proprietary feed medium on days 3, 7, and 10. Viable cell count (VCC) and percent viability of cells in culture was measured on days 0, 3, 7, 10, and 14 using a Cedex HiRes (Roche Diagnostics GmbH, Mannheim, Germany). Glucose concentration, lactate concentration and product titer were measured on days 3, 5, 7, 10, 12 and 14 using a Cobas analyzer (Roche Diagnostics GmbH, Mannheim, Germany). The supernatant was harvested 14 days after start of fed-batch by centrifugation (10 min., 1000 rpm followed by 10 min., 4000 rpm) and cleared by filtration (0.22 μm). Day 14 titers were further determined using protein A affinity chromatography with UV detection. Product quality was determined by Caliper's LabChip (Caliper Life Sciences).

### Example 9

### High Cell Density Fed-Batch Cultivation

Fed-batch production cultures were performed in Ambr 15 or Ambr 250 vessels (Sartorius Stedim) with proprietary chemically defined medium. Cells were seeded at 15×10E6 cells/ml on day 0. Cultures received proprietary feed medium on days 1, 3, and 6. Viable cell count (VCC) and percent viability of cells in culture was measured on days 0, 3, 7, 10, and 14 using a Cedex HiRes instrument (Roche Diagnostics GmbH, Mannheim, Germany). Glucose concentration, lactate concentration and product titer were measured on days 3, 5, 7, 10, 12, and 14 using a Cobas Analyzer (Roche Diagnostics GmbH, Mannheim, Germany). The supernatant was harvested 14 days after start of the cultivation by centrifugation (10 min., 1000 rpm followed by 10 min., 4000 rpm) and cleared by filtration (0.22 μm). Day 14 titers were further determined using protein A affinity chromatography with UV detection. Product quality was determined by Caliper's LabChip (Caliper Life Sciences).

1. A method for increasing heterologous polypeptide expression of a recombinant mammalian cell comprising an exogenous nucleic acid encoding a heterologous polypeptide by reducing expression of at least the endogenous gene MYC, compared to a mammalian cell cultivated under the same conditions that has the identical genotype but endogenous expression of said expression-reduced gene.

2. A method for producing a heterologous polypeptide comprising the steps of
a) cultivating a mammalian cell comprising a deoxyribonucleic acid encoding the heterologous polypeptide, and
b) recovering the heterologous polypeptide from the cell or the cultivation medium,
wherein the expression of at least the endogenous gene MYC has been reduced or knocked-out in said mammalian cell.

3. A method for producing a recombinant mammalian cell with improved recombinant productivity, wherein the method comprises the following steps:
a) applying a nuclease-assisted and/or nucleic acid targeting the endogenous gene MYC in a mammalian cell to reduce the activity of said endogenous gene, and
b) selecting a mammalian cell wherein the activity of said endogenous gene MYC has been reduced or knocked out,
thereby producing a recombinant mammalian cell with increased recombinant productivity compared to a compared to a mammalian cell cultivated under the same conditions that has the identical genotype but endogenous expression of said gene.

4. The method according to claim 2, wherein the gene knockout is a heterozygous knockout or a homozygous knockout.

5. The method according to claim 2, wherein the productivity of the modified cell is at least 10% increased compared to the parent mammalian cell with the same genotype except for the reduction or the knockout of said gene.

6. The method according to claim 2, wherein the reduction gene expression is mediated by a nuclease-assisted gene targeting system.

7. The method according to claim 6, wherein the nuclease-assisted gene targeting system is selected from the group consisting of CRISPR/Cas9, CRISPR/Cpf1, zinc-finger nuclease, TALEN and meganuclease.

8. The method according to claim 2, wherein the reduction of gene expression is mediated by RNA silencing.

9. The method according to claim 8, wherein RNA silencing is selected from the group consisting of siRNA gene targeting and knock-down, shRNA gene targeting and knock-down, and miRNA gene targeting and knock-down.

10. The method according to claim 2, wherein the heterologous polypeptide is an antibody.

11. The method according to claim 2, wherein the knockout is performed before the introduction of the exogenous nucleic acid encoding the heterologous polypeptide or after the introduction of the exogenous nucleic acid encoding the heterologous polypeptide.

12. The method according to claim 2, wherein the mammalian cell is a CHO cell.

13. The method according to claim 2, wherein the expression of the endogenous genes SIRT-1 and MYC has been reduced.

14. The method according to claim 3, wherein the gene knockout is a heterozygous knockout or a homozygous knockout.

15. The method according to claim 3, wherein the productivity of the modified cell is at least 10% increased compared to the parent mammalian cell with the same genotype except for the reduction or the knockout of said gene.

17. The method according to claim 3, wherein the reduction of gene expression is mediated by a nuclease-assisted gene targeting system, and wherein the nuclease-assisted gene targeting system is selected from the group consisting of CRISPR/Cas9, CRISPR/Cpf1, zinc-finger nuclease, TALEN and meganuclease.

18. The method according to claim 3, wherein the reduction of gene expression is mediated by RNA silencing, and wherein RNA silencing is selected from the group consisting of siRNA gene targeting and knock-down, shRNA gene targeting and knock-down, and miRNA gene targeting and knock-down.

19. The method according to claim 3, wherein the knockout is performed before the introduction of the exogenous nucleic acid encoding the heterologous polypeptide or after the introduction of the exogenous nucleic acid encoding the heterologous polypeptide.

20. The method according to claim 3, wherein the mammalian cell is a CHO cell.
